SEQUENCE LISTING (38 sequences)

SEQ ID NO: 1 - DNA, 20 bp, Artificial Sequence
  Description of Artificial Sequence/note = synthetic construct
gcctattggtctattttccc

SEQ ID NO: 2 - DNA, 18 bp, Artificial Sequence
  Description of Artificial Sequence/note = synthetic construct
cctgaagttctcaggatc

SEQ ID NO: 3 - DNA, 32 bp, Artificial Sequence
  Description of Artificial Sequence/note = synthetic construct
atctggcaccacaccttctacaatgagctgcg

SEQ ID NO: 4 - DNA, 32 bp, Artificial Sequence
  Description of Artificial Sequence/note = synthetic construct
cgtcatactcctgcttgctgatccacatctgc

SEQ ID NO: 5 - DNA, 19 bp, Artificial Sequence
  Description of Artificial Sequence/note = synthetic construct
tgcaaggagtttcatcctg

SEQ ID NO: 6 - DNA, 21 bp, Artificial Sequence
  Description of Artificial Sequence/note = synthetic construct
agaatcagtagtttaacacac

SEQ ID NO: 7 - DNA, 22 bp, Artificial Sequence
  Description of Artificial Sequence/note = synthetic construct
tgagcatagttattaatagcag

SEQ ID NO: 8 - DNA, 77 bp, Artificial Sequence
  Description of Artificial Sequence/note = synthetic construct
gctagctcgagaccggtgccaccatggactacaaagacgatgacgacaaggcggaagggctggagcgtgtgcggatc

SEQ ID NO: 9 - DNA, 43 bp, Artificial Sequence
  Description of Artificial Sequence/note = synthetic construct
tttaaacccggcctgcggggccagagtagccaggatcccgcgc

SEQ ID NO: 10 - DNA, 18 bp, Artificial Sequence
  Description of Artificial Sequence/note = synthetic construct
tgaccttcagcgcctcgg

SEQ ID NO: 11 - DNA, 17 bp, Artificial Sequence
  Description of Artificial Sequence/note = synthetic construct
ctccgagtccctctgcc

SEQ ID NO: 12 - DNA, 19 bp, Artificial Sequence
  Description of Artificial Sequence/note = synthetic construct
ggcaaaggctctgagaagc

SEQ ID NO: 13 - DNA, 17 bp, Artificial Sequence
  Description of Artificial Sequence/note = synthetic construct
ccgaggtcccaaaggcg

SEQ ID NO: 14 - DNA, 39 bp, Artificial Sequence
  Description of Artificial Sequence/note = synthetic construct
atcgaagatctggatccaaggtcgggcaggagagggcct

SEQ ID NO: 15 - DNA, 59 bp, Artificial Sequence
  Description of Artificial Sequence/note = synthetic construct
tacacaaagcaatgtccattacatgccacggtgtttcgtcctttccacaagatatataa

SEQ ID NO: 16 - DNA, 48 bp, Artificial Sequence
  Description of Artificial Sequence/note = synthetic construct
cgaaatctagaaaaaagtggcatgtaatggacattgcctacacaaagc

SEQ ID NO: 17 - DNA, 21 bp, Artificial Sequence
  Description of Artificial Sequence/note = synthetic construct
gcugcagcagaacaggccatt

SEQ ID NO: 18 - DNA, 21 bp, Artificial Sequence
  Description of Artificial Sequence/note = synthetic construct
guacaacccaggauaugugtt

SEQ ID NO: 19 - DNA, 59 bp, Artificial Sequence
  Description of Artificial Sequence/note = synthetic construct
tacacaaacagggctgttcttcgagatgcggtgtttcgtcctttccacaagatatataa

SEQ ID NO: 20 - DNA, 48 bp, Artificial Sequence
  Description of Artificial Sequence/note = synthetic construct
cgaaatctagaaaaaagcatctcgaagaacagccctgctacacaaaca

SEQ ID NO: 21 - DNA, 59 bp, Artificial Sequence
  Description of Artificial Sequence/note = synthetic construct
tacacaaagcaatgtccgttgcatgccacggtgtttcgtcctttccacaagatatataa

SEQ ID NO: 22 - DNA, 48 bp, Artificial Sequence
  Description of Artificial Sequence/note = synthetic construct
cgaaatctagaaaaaagtggcatgcaacggacattgcctacacaaagc

SEQ ID NO: 23 - DNA, 59 bp, Artificial Sequence
  Description of Artificial Sequence/note = synthetic construct
tacacaaagttcagaggctgtgtcataacggtgtttcgtcctttccacaagatatataa

SEQ ID NO: 24 - DNA, 48 bp, Artificial Sequence
  Description of Artificial Sequence/note = synthetic construct
cgaaatctagaaaaaagttatgacacagcctctgaacctacacaaagt

SEQ ID NO: 25 - DNA, 59 bp, Artificial Sequence
  Description of Artificial Sequence/note = synthetic construct
tacacaaaccaaggcacttgttggcagtcggtgtttcgtcctttccacaagatatataa

SEQ ID NO: 26 - DNA, 48 bp, Artificial Sequence
  Description of Artificial Sequence/note = synthetic construct
cgaaatctagaaaaaagactgccaacaagtgccttggctacacaaacc

SEQ ID NO: 27 - DNA, 20 bp, Artificial Sequence
  Description of Artificial Sequence/note = synthetic construct
gcagcgagcaactgagaagc

SEQ ID NO: 28 - DNA, 22 bp, Artificial Sequence
  Description of Artificial Sequence/note = synthetic construct
gggtttagtggtacttgtgagc

SEQ ID NO: 29 - DNA, 19 bp, Artificial Sequence
  Description of Artificial Sequence/note = synthetic construct
gactgagccgatcccgcgc

SEQ ID NO: 30 - DNA, 20 bp, Artificial Sequence
  Description of Artificial Sequence/note = synthetic construct
gcagtaacggcagacttctc

SEQ ID NO: 31 - DNA, 19 bp, Artificial Sequence
  Description of Artificial Sequence/note = synthetic construct
cctttcctgctcttgcctg

SEQ ID NO: 32 - DNA, 23 bp, Artificial Sequence
  Description of Artificial Sequence/note = synthetic construct
gctttttatttgtcagaagacag

SEQ ID NO: 33 - DNA, 30 bp, Artificial Sequence
  Description of Artificial Sequence/note = synthetic construct
atctggcaccacaccttctacaatgagctg

SEQ ID NO: 34 - DNA, 30 bp, Artificial Sequence
  Description of Artificial Sequence/note = synthetic construct
cgtcatactcctgcttgctgatccacatct

SEQ ID NO: 35 - DNA, 18 bp, Artificial Sequence
  Description of Artificial Sequence/note = synthetic construct
atgacttcgaaagtttat

SEQ ID NO: 36 - DNA, 19 bp, Artificial Sequence
  Description of Artificial Sequence/note = synthetic construct
ttcagatttgatcaacgca

SEQ ID NO: 37 - protein, 1419 aa, Artificial Sequence [sequence omitted]

SEQ ID NO: 38 - DNA, 5965 bp, Artificial Sequence
  Description of Artificial Sequence/note = synthetic construct
cctggctgcgcgcggcggtggcggagccgctacggctgtagcagcagccgcgaagatggcggaagggctggagcgtgtgcggatctccgcgtcggagctgcgcgggatcctggctactctggccccgcaggccgggagcagagaaaacatgaaggaattaaaggaggccaggccgcgcaaagataacaggcgtccagatctggaaatctataagcctggcctttctcggctaaggaacaagcccaaaatcaaggaaccccctgggagtgaggaattcaaagatgaaattgttaatgaccgagattgctctgctgttgaaaatggtacacagcccgttaaagatgtctgcaaggaactgaacaaccaagagcagaatggtcctatagacccagaaaataatcggggacaagaatcctttcctaggactgctggacaagaggatcgtagtctaaaaattatcaaaagaacaaagaaacccgacctgcagatctatcagcctggacgacgtttgcagactgttagcaaagaatccgccagtcgggtggaggaggaagaagtcctcaaccaggtagaacaactgagagtagaggaagatgagtgtaggggaaatgttgcgaaggaggaagttgcgaataaaccagacagggccgagatagaaaagagcccaggtggtgggagagtaggggctgcaaaaggagaaaaaggaaagaggatgggaaaaggggagggggtgagggaaacccacgacgacccggcccgcgggaggccgggctccgcaaagcgctactcccgctcagacaaacgaaggaatcgctaccgcacgcgcagcaccagctcagctggcagcaacaacagcgctgagggagctggcctgacggataatggatgtcgccgccgccgacaggataggaccaaggagaggccaccactgaagaagcaagtgtctgtgtcctcaaccgattccttagacgaggacagaattgatgagcctgatggattaggacccaggagaagttcagaaaggaagagacatttagaaagaaactggtctggccgtggggagggtgagcagaaaaccagtgctaaagaatatcgaggcactcttcgtgtcactttcgatgcagaagccatgaacaaagagtctcccatggtgaggtcagccagggatgatatggatagaggaaagcctgacaaaggcttgagcagtgggggcaaaggctctgagaagcaggagtccaaaaacccgaaacaagaacttcggggtcgtggtcgtggcattctgattttgcctgcccataccaccctatctgtcaattcagcaggttctccagagtccgcgcctttgggacctcggcttttgtttggatctggtagtaagggatctcggagttggggccgtggaggcaccacacgccgattgtgggacccaaacaatcctgatcagaaacctgctctaaagactcagacgccccagctacatttcttggacactgatgatgaagtcagccctacatcttggggtgactcacgccaggctcaggcatcttactataagtttcaaaactctgacaacccctattattacccccggacaccaggccctgcctcccagtatccctatacgggctataaccctctacagtacccagtgggccctacgaatggtgtgtacccagggccttactacccaggctacccgactccgtcaggacagtatgtgtgtagccctctacctaccagcaccatgagtcccgaggaggtagagcagcacatgaggaacctgcagcaacaggagctgcacaggcttctccgggtggctgacaaccaggaactgcagctcagcaacctgctctccagggaccgcatcagtccggagggcctggagaagatggcgcaactcagagctgaactgctgcagctatatgagcgctgtattctattagatattgagttctctgataatcagaatgtggatcagatcctgtggaagaatgctttctatcaggtgattgagaagttcaggcaacttgtcaaggatccgaatgttgagaacccagaacagattcggaacagacttttggagctcttggatgagggtagtgacttctttgatagtttgcttcagaagctgcaggttacttacaagttcaaactggaagactacatggatggtcttgccattcgcagcaagccattacgcaagacagtaaaatatgccttgatcagtgcccagcgatgcatgatatgccaaggagatattgctaggtaccgggagcaagccagtgatacagcgaattatgggaaagcacgcagttggtacctgaaggcccagcacattgctcccaagaatgggcgcccctataaccagttggctttgctggcagtgtatacgaggaggaagcttgacgctgtctattactatatgcgcagtttagctgccagcaaccctatcctgactgccaaggagagtctcatgagcttgtttgaagagaccaagcggaaggcagaacagatggaaaagaagcaacatgaggaatttgacctgagccctgaccagtggcggaaaggaaagaagtctactttccggcatgttggagatgacaccactcgcctggagatctggattcatccatcccatccacggtcttcccagggcactgagtctgggaaggattctgagcaagagaatgggctgggcagcctgagtcccagtgatctgaacaaaaggttcatcctcagttttctccatgcccatgggaagctgtttacccggattgggatggagacattccctgcagtggctgagaaggtcctcaaggagttccaggtgttactgcagcacagcccctctcccattggaagtacccgcatgctgcagcttatgaccatcaatatgtttgcagtacacaactcccagctgaaagactgcttctcggaggagtgccgctctgtgatccaggaacaagccgcagctctgggcttggccatgttttctctactggtccgccgctgcacctgcttacttaaggagtccgccaaagctcagctgtcctctcctgaggaccaggatgaccaagacgacatcaaggtgtcttcctttgtcccggacctgaaggagctgctccccagtgtcaaagtctggtcagattggatgctcggctacccggacacctggaatcctcctcccacatccctggatctgccctcgcatgttgctgtggatgtatggtcgacgctggctgatttctgtaacatactgactgcagtgaatcagtctgaggtgccactgtacaaggacccggatgatgacctcacccttcttatcctggaagaggatcggcttctctcgggctttgtccccttgctggctgcccctcaggacccctgctacgtggagaaaacctcggataaggttattgcagctgactgcaaaagggtcacagtgctgaagtattttctggaagccctttgtggacaagaagagcctctgctggcattcaagggtggaaagtatgtgtcagtggcacccgtcccagacaccatgggaaaggaaatgggaagccaagagggaacacgactggaagatgaggaggaggatgtggtgattgaagactttgaggaagattcagaggctgaaggcagcggaggcgaggatgacatcagggagcttcgggccaagaagctggctctggccaggaagatagctgagcagcagcgtcgccaggaaaagatccaggctgtcctggaggaccacagtcagatgaggcagatggagctcgaaatcagacctttgttcctcgtaccagacaccaacggcttcattgaccacctggccagtctggcgcggctgctggagagcaggaagtacatcctggtggtgcccctcatcgtgatcaatgagctggacggcctggccaaggggcaggagacagaccaccgggctgggggctacgcccgtgtggtacaagagaaggcccgcaagtccatcgagttcctcgagcagcgattcgagagtcgggactcttgcctgcgagccctgaccagccgtggcaatgaactcgaatccatcgccttccgcagtgaggacatcactggccagctgggtaacaacgatgatctcatcctgtcctgctgcctccactactgcaaagacaaggctaaggacttcatgcccgccagcaaagaggagccaatccggctactgcgggaggtggtgctgttgacggatgaccggaacctgcgtgtgaaggcgctcacaaggaatgttcctgtacgggacatcccagccttcctcacgtgggcccaggtgggctgagggagccacactggggccccccccccccgtggaaccgttcctgaaaggccaccaggcgcccagtgtagcacggaagatgcccacgtgcctgagccaccaatccacccagacaataaaccatcctcttccaacccacgccacggccatgctgtgggggacctgctcctcacagagcccctcccaaggatcgggcggaagctgctgggaccctcctgggctgccaggatttagcagggaggtggctggctacagcaacagcagctgggcaagccagataggccgcccatgctctcagcctttctccctcccccgtctcattccaaggctgagggagggccttctcgcctggggacgcagccactttctccagtggagacagggcaggggttcagagtttccgtcagatgcagtgaaatcacagttccctttcatcttcagaacctctgtcgtgaatgtgttcaagaggctttggttaagtcaggaagaagtgcccagggtgtgtgtccccagtctccctgaggcctggactcgcccatgaacccaagtcggcttctagacagcatgtccctaacagcagccctgggcccccacctcttctaccatccaccccagacttaccacacacccttcctgctgctcctcttcctgcccttatcaacctgggtccctcacacttcgccagttgcgtccccgtggacagtcatgagtctagaggaaaggggcatctggtctcaggcccgtgctctcgggtggcctccacctgctccctttctcctcactggcctttctttccgtctagcctcctcttcaggaaatgtcctgactctcctcagctcccccttcacccctccttgcccgcctaccctccctccagaatagcccctcacccttcttccccttctagttgatccttttcacctccctgatccccttcatttcttcaccgcggttcctcgtcataggggttctcactctgaactttccctctctactacccatggcaggaacctagtacaggtctcccacccagggccttccaccctcggctcctgtgctgggagaaacttccaggcgtggacagcccagcctgaggcattccagtgctggggcaccgtcgcctaacctggtttctagctttgccctcactccccggaaaaactgacactgacacaggggccctttccttgcccctttagctggtacctcagtggggaggcttccttaccaagaatgagttcctgaaacccagggccagagacaaggacaacttaggggaagacggggttttcggtggagccaggggcaaatcttaatgggaccagtgggggataccccagagcccatggcctgactgcacagcctgcctggaggatgggtgcgcagctctgccctccctgaggcccaggactatgccagaagcgatggggtaccgtgtaggggagccaaggccagtagtttgggggtaggagtcccctagagtctcagaagactgggctctttggagtacagggtccccggcctctcctttaagattctctccccagctggaaggccgatgactgggtggtcgggagggagacccagctctcctttctgtcccgtttgcagcactggttttgtttccttaataaatttttagttatgaaacat

(19) United States
(12) Patent Application Publication
(10) Pub. No.: US 2006/0154883 A1
(43) Pub. Date: Jul. 13, 2006

(54) Nonsense-mediated mrna decay

(76) Inventor: Lynne E Maquat, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/525,273

(22) PCT Filed: Aug. 21, 2003
(86) PCT No.: PCT/US03/26166

§ 371 Date: Feb. 7, 2006

Related U.S. Application Data
(60) Provisional application No. 60/405,602, filed on Aug. 22, 2002.

(51) Int. Cl. A61K 48/00

(52) U.S. Cl. 514/44

(57) ABSTRACT

Disclosed are compositions and methods relating to nonsense mediated decay (NMD) particularly in the “pioneer” round of translation. The present invention provides methods of treating disorders relating to NMD and methods of screening substances for use in the study of or treatment of conditions relating to NMD.

This application claims benefit of U.S. Provisional Application No. 60/405,602 filed on Aug. 22, 2002, which is incorporated herein in its entirety. This invention was made with government support under Grants NIH DK33938 and NIH GM59614 awarded by NIH. The government has certain rights in the invention.

### I. BACKGROUND OF THE INVENTION

1. In mammalian cells, the expression of protein-encoding genes requires a series of steps in which pre-mRNA is processed to mRNA in the nucleus before mRNA is translated into protein in the cytoplasm. These steps are subject to quality control to ensure that only completely processed mRNA is exported to the cytoplasm (reviewed in Maquat and Carmichael, 2001). One form of quality control, called mRNA surveillance or nonsense-mediated mRNA decay (NMD), degrades mRNAs that prematurely terminate translation more than 50-55 nucleotides upstream of an exon-exon junction as a means to prevent the synthesis of potentially harmful truncated proteins (reviewed in Maquat, 1995, 2006, Li and Wilkinson, 1998; Culbertson, 1999; Hentze and Kulozik, 1999; Wilusz and Peltz, 2001). Beside translation, NMD in mammalian cells requires at least four proteins: Upf1, Upf2, Upf3/3X (related products of different genes that are also called Upf3a/b) and hSMG1/ATX (Sun et al., 1998; Mendell et al., 2000; Lykke-Andersen et al., 2000; Serin et al., 2001; Denning et al., 2001; Yamashita et al., 2000). Upf1, Upf2, Upf3 and Upf3X interact in a way where Upf1 binds directly to Upf2, and Upf2 binds directly to Upf3 and Upf3X. Studies of Upf orthologues in S. cerevisiae indicate that Upf1 interacts with eukaryotic release factors (eRFs) 1 and 3, Upf2 and Upf3 interact with eRF3 in a way that competes with the eRF3-eRF1 interaction, and all three proteins influence translation termination efficiency (Czaplinski et al., 1998; Maderazo et al., 2000; Wang et al., 2001). hSMG1/ATX, like its orthologue in C. elegans (Page et al., 1999), is a phosphatidyl inositol 3-kinase-related protein kinase involved in the phosphorylation of Upf1 (Pal et al., 2001; Denning et al., 2001; Yamashita et al., 2001).

2. Exon-exon junctions have been proposed to function in NMD via the −335 kDa exon junction complex (EJC) of proteins that are deposited −20-24 nucleotides upstream of junctions as a consequence of pre-mRNA splicing (Le. Hir et al., 2000a&b, 2001c; Kim et al., 2001b; Lykke-Andersen et al., 2001; Kataoka et al., 2001). Components of this complex include REF/Aly, Y14, DEK, SRm160, and RNPS1. REF/Aly facilitates the nuclear export of mRNA by interacting with the mRNA export receptor TAP (Lou and Reed, 1999; Zhou et al, 2000; Stutz et al., 2000; Rodrigues et al., 2001; Katahira et al., 1999; Bachi et al., 2000; Kataoka et al., 2000, 2001; Le Hir et al., 2001a). Y14, which binds to mRNA that has undergone splicing (Kataoka et al., 2000) and interacts with REF/Aly and RNPS1 in vitro Kataoka et al., 2001), has been proposed to provide a position-specific memory of the EJC in the cytoplasm since it is detected in association with both nuclear and newly exported cytoplasmic mRNA (Kim et al., 2001b). DEK has multiple functions that include interacting with SR proteins during splicing (McGarvey et al., 2000) as well as altering the superhelical density of DNA in chromatin (Alexiadis et al., 2000) and altering the transcription of certain genes (Faulkner et al., 2001). SRm160 co-activates pre-mRNA splicing (Blencowe et al., 1998; McGarvey et al., 2000; Kataoka et al., 2000) and promotes transcript 3′-end cleavage (McCracken et al., 2002). Notably, neither DEK nor SRm160 shuttle to the cytoplasm in assays using mammalian-cell heterokaryons (Lykke-Andersen et al., 2001; Y.I.). RNPS1 functions in pre-mRNA splicing (M4ayeda et al., 1999) and was recently shown to mechanistically connect splicing and NMD (Lykke-Andersen et al., 2001): (i) RNPS1 and, to a lesser extent, Y14 tethered to the 3′ untranslated region of β-globin mRNA recapitulate the function of the EJC in NMD as does tethered Upf1, Upf2 or Upf3/3X (Lykke-Andersen et al., 2000; 2001), and (ii) FLAG-RNPS1 transiently expressed in HEK293 cells co-immunoprecipitates with Upf1, Upf2 and Upf3/3X (Lykke-Andersen et al., 2001). Considering that Upf3/3X, RNPS1 and Y14 are mostly nuclear but shuttle to the cytoplasm, Upf2 is cytoplasmic but primarily perinuclear, and Upf1 is primarily cytoplasmic (Lykke-Andersen et al., 2000, 2001; Serin et al., 2001), these data indicate that Upf3/3X joins the splicing-dependent mRNP complex in the nucleus by either directly or indirectly interacting with RNPS1 and, possibly, Y14 (Lykke-Andersen et al., 2001). Extending the idea that Upf3/3X is recruited by the complex, Y14 has been shown to interact with REF/Aly, TAP and Upf3/3X independently of RNA, and Upf3X has been shown to map in vivo upstream of the exon-exon junction of two spliced mRNAs (Kim et al., 2001a). According to current thinking, Upf2 joins the complex during or immediately after export to the cytoplasm. Provided that translation terminates prematurely (i.e., more than 50-55 nucleotides upstream of an EJC-marked exon-exon junction), Upf1 subsequently interacts with the complex in a way that elicits NMD (Lykke-Andersen et al., 2001; Ishigaki et al., 2001).

3. Another important connection between splicing and NMD was recently elucidated with the finding that Upf2 and Upf3X can be detected on spliced mRNP immunopurified using anti-CBP80 antibody but not anti-eIF4E antibody (Ishigaki et al., 2001). This result implies that the two Upf proteins are recruited to newly synthesized mRNA that has yet to be translated. CBP80 is the mostly nuclear cap binding protein that shuttles in association with mRNA to the cytoplasm (Visa et al., 1996; Shen et al., 2000; Ishigaki et al., 2001), where it is replaced by the mostly cytoplasmic cap binding protein eukaryotic initiation factor (eIF) 4E (reviewed in Gingras et al., 1999). CBP80-bound mRNA, rather than eIF4E-bound mRNA, was found to be the primary template for the first so-called “pioneer” round of translation since it is the primary substrate of NMD (Ishigaki et al., 2001). The conclusion that CBP80-bound mRNA is targeted for NMD was based on several criteria: (i) nonsense-containing CBP80-bound mRNA is reduced in abundance to an extent that is comparable to nonsense-containing eIF4E-bound mRNA, and CBP80-bound mRNA is thought to be a precursor to eIF4E-bound mRNA, (ii) the nonsense-mediated reduction in the abundance of CBP80-bound mRNA is abrogated by either a suppressor tRNA that recognizes the nonsense codon as encoding an amino acid or a cycloheximide-induced block in translation, (iii) Upf2 and Upf3X are detected on CBP80-bound mRNA but not eIF4E-bound mRNA, and (iv) mRNA immunopurified using anti-Upf3/3X antibody is bound by CBP80 but not eIF4E (Ishigaki et al., 2001). These data indicate that NMD is generally confined to CBP80-bound mRNA, before CBP80 is replaced by eIF4E, and before Upf2 and Upf3X dissociate from the vicinity of splicing-generated exon-exon junctions (Ishigaki et al., 2001).

### II. SUMMARY OF THE INVENTION

4. In accordance with the purposes of this invention, as embodied and broadly described herein, this invention, in one aspect, relates to methods of treating subjects with conditions resulting from nonsense-mediated mRNA decay, screening for, and manufacturing those therapeutic agents.

5. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

6. The accompanying drawings, which are incorporated in and constitute a part of this specification, illustrate several embodiments of the invention and together with the description, serve to explain the principles of the invention.

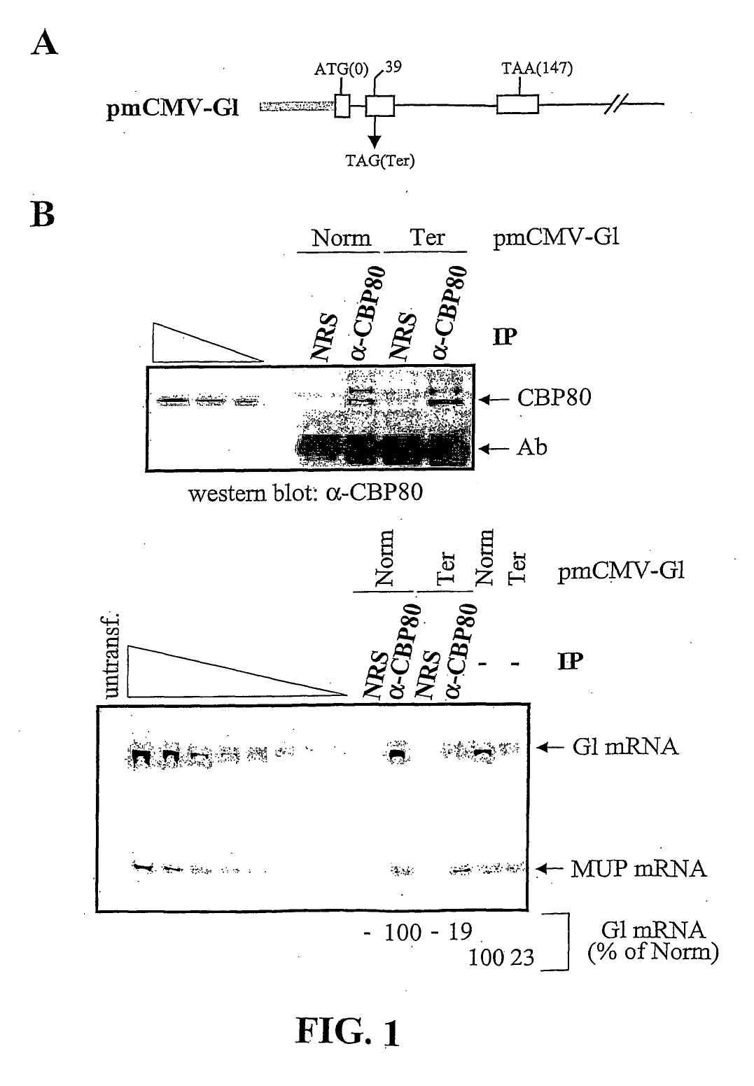

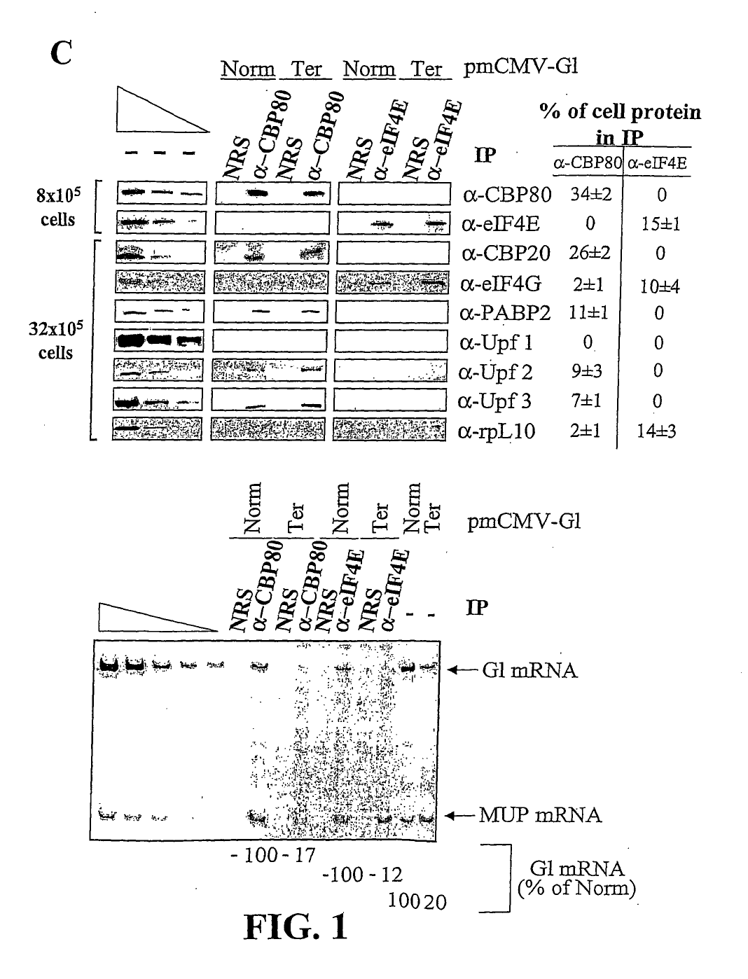

7. FIG. 1 shows nonsense-containing ∃-globin (Gl) mRNP immunopurified using anti-CBP80 antibody is a target for NMD. (A) Structure of the pmCMV-Gl test plasmids, which harbored either a nonsense free (Norm) Gl allele or a TAG nonsense codon at position 39 (Ter). Cos cells were transiently transfected with a test pmCMV-Gl plasmid and the reference plasmid phCM-MUP. (B, upper) Total-cell RNP was then immunopurified using either normal rabbit serum (NRS) or anti-CBP80 antibody (∀-CBP80) and subjected to western blotting using anti-CBP80 antibody. The three left-most lanes analyzed decreasing amounts of protein, demonstrating that the conditions used for western blotting were quantitative. (B, lower) RNA was purified from total-cell RNP either before (−) immunopurification (IP) or after immunopurification (NRS or ∀-CBP80). The levels of Gl and MUP mRNAs were then analyzed by RT-PCR. The left-most lane demonstrates the absence of either mRNA in untransfected cells, and the adjacent eight lanes analyzed decreasing amounts of RNA, demonstrating that the conditions of RT-PCR were quantitative. (C, upper) As in B (upper) except that immunopurifications were scaled up (each IP lane derives from either 8×10⁵ or 32×10⁵ cells) and also performed with anti-eIF4E antibody, and western blotting also utilized anti-eIF4E, anti-CBP20, anti-eIF4G, anti-PABP2, anti-human Upf1, anti-human Upf2, anti-human Upf3/3X, and ribosomal protein (rp) L10 antibodies. Furthermore, the percent (%) of cellular protein in each IP (tabulated to the right) was determined by comparing the western blot intensity of each IP lane to the intensities of 3-fold dilutions of extract from either 8×10⁵ or 32×10⁵ cells before IP (three left-most lanes). Protein detectably reacting with anti-Upf3/3X antibody is likely to be Upf3 protein rather than Upf3X protein (which derives from the X-linked UPF3 gene that is related to the UPF3 gene) since it co-migrated with the HeLa-cell Upf3 protein. The Upf3X isoform likely co-migrated with antibody, making it undetectable using electrophoresis in either one or two dimensions. (C, lower) As in B (lower) except that RNA analysis was extended to immunopurifications using anti-eIF4E antibody. Each panel of results is representative of five independently performed experiments where the efficiency of NMD varied by no more than 4% (see text for specific standard deviations).

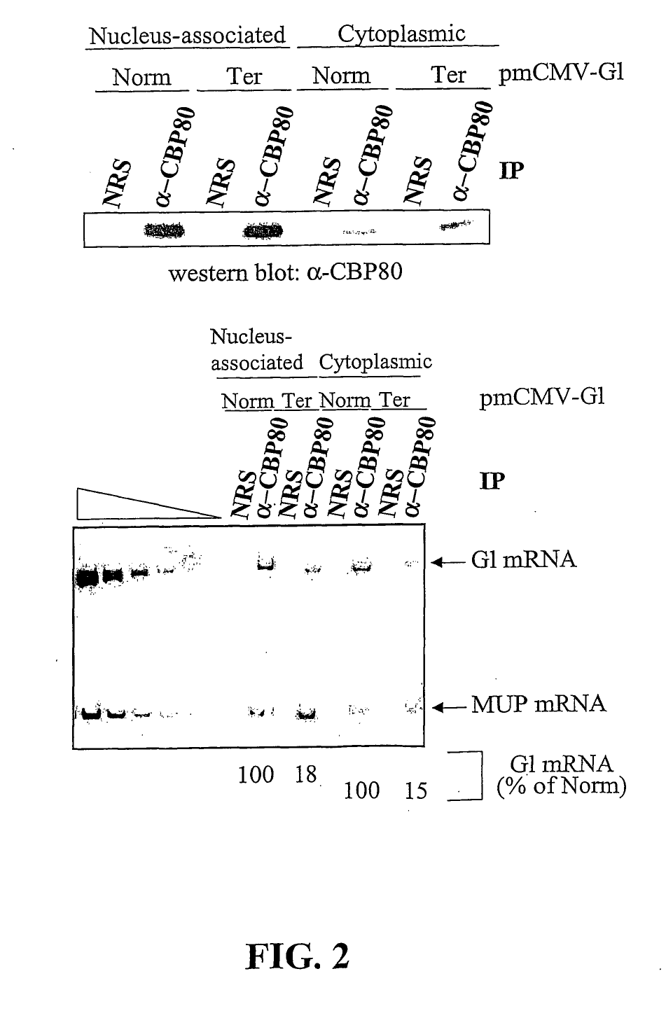

8. FIG. 2 shows the NMD of CBP80-bound Gl mRNA is nucleus-associated. As in FIG. 1B except that nuclear and cytoplasmic fractions were immunopurified with NRS and anti-CBP80 antibody. Nuclear fractions were free of cytoplasm as evidenced by the absence of detectable reactivity with anti-eIF4A antibody. The amount of reactivity obtained with anti-CPB80 antibody after immunopurification of nuclear and cytoplasmic fractions can be quantitatively compared, as evidenced by analyzing decreasing amounts of protein and demonstrating that the conditions used for western blotting were quantitative. The efficiency of NMD varied by no more than 2% in two independently performed transfections.

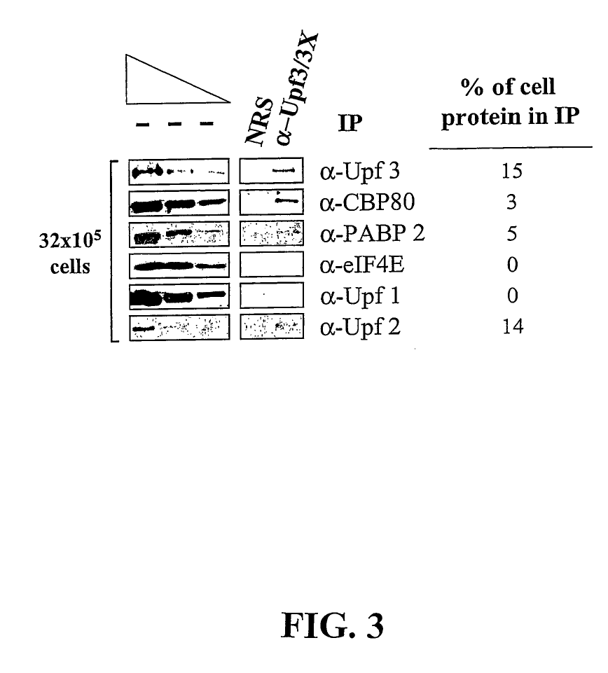

9. FIG. 3 shows RNP immunopurified using anti-Upf3/3X antibody contains CPB80, PABP2 and Upf2 protein but not eIF4E or Upf1 protein. As in FIG. 1C (upper) except that cells were not transfected and immunopurifications were performed with anti-Upf3/3X antibody. Results are representative of three independently performed immunopurifications.

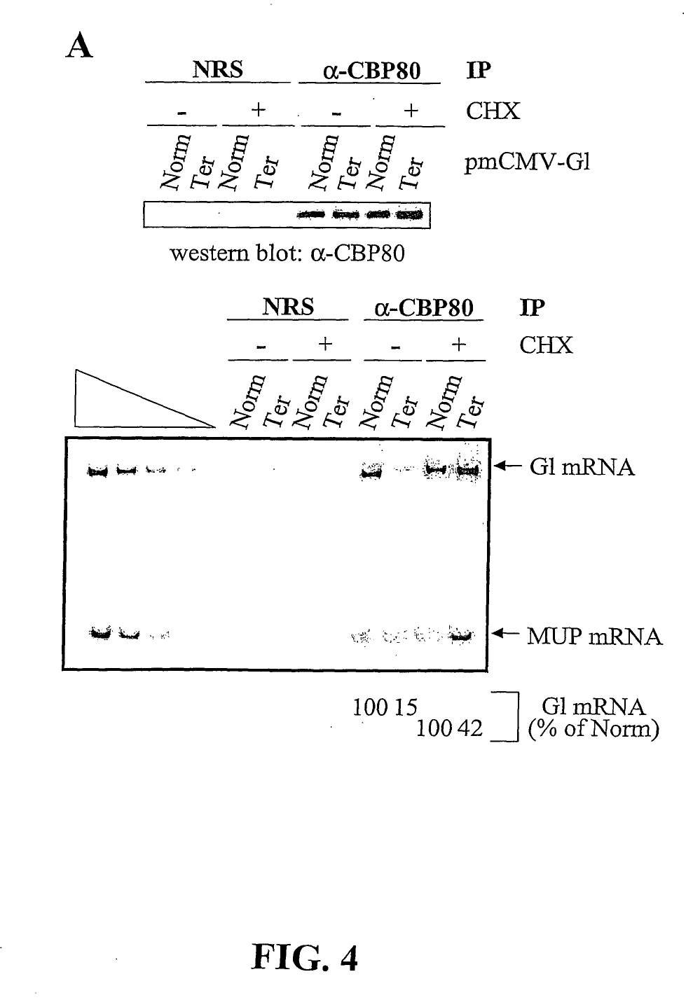

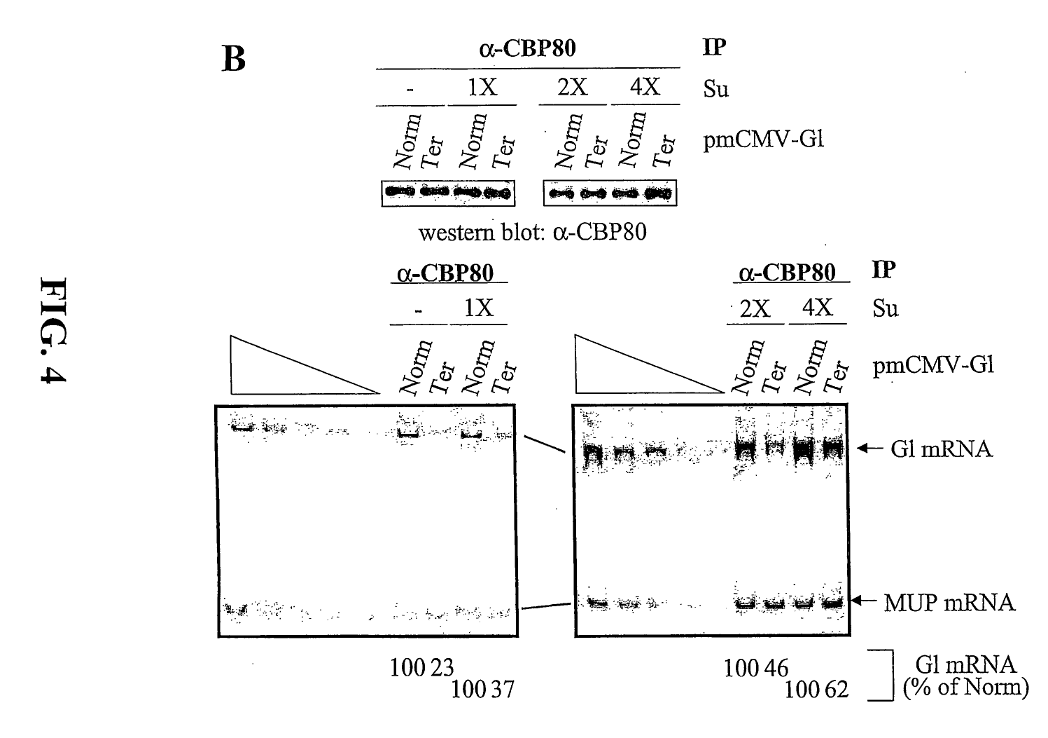

10. FIG. 4 shows cycloheximide and suppressor tRNA abrogate the NMD of CBP80-associated Gl mRNA, providing evidence that CBP80-associated mRNA is translated. (A) As in FIG. 1B except that cycloheximide was (+CHX) or was not (−CHX) added two hr prior to cell harvest. (B) As in FIG. 1B except that a derivative of p53tsSu⁺ that harbored the amber suppressor tRNA in either antisense orientation (−Su) or sense orientation at increasing concentrations (1×, 2× and 4× Su) was included in the transfection, and immunopurifications using NRS were not performed. Results are representative of two independently performed experiments.

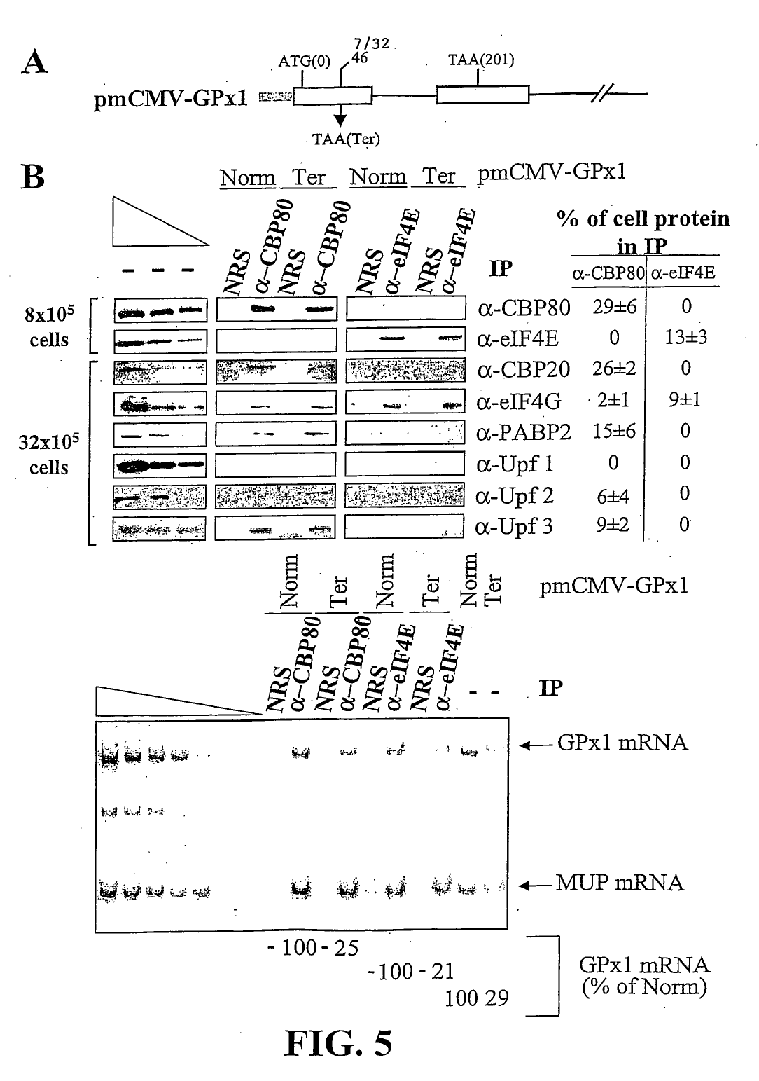

11. FIG. 5 shows nonsense-containing glutathione peroxidase1 (GPx1) mRNA immunopurified using anti-CBP80 antibody is subject to NMD. (A) Structure of the pmCMV-GPx1 test plasmids, which harbored either a nonsense-free (Norm) GPx1 allele or a TAA nonsense codon at position 46 (Ter). (B) As in FIG. 1C except that GPx1 mRNA was analyzed in place of Gl mRNA. Each panel of results is representative of three independently performed experiments, and the efficiency of NMD varied by no more than 5% (see text for specific standard deviations).

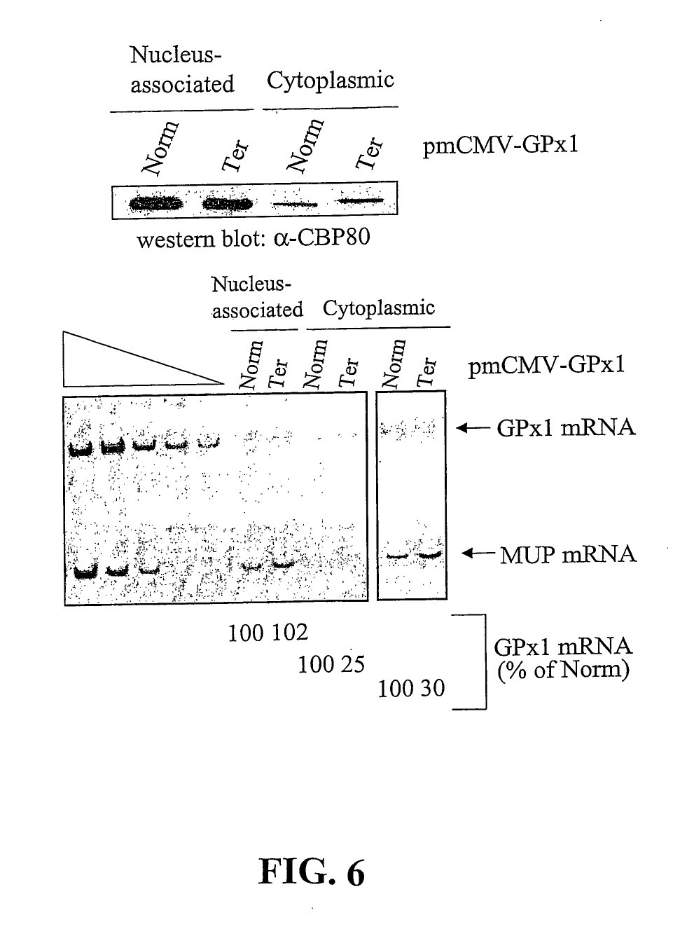

12. FIG. 6 shows the NMD of CBP80-bound GPx1 mRNA is cytoplasmic. As in FIG. 2 except that GPx1 mRNA was analyzed in place of Gl mRNA, and the analysis of NRS was omitted. The efficiency of NMD varied by no more than 5% in two independently performed experiments. The right-hand box represents a separate and more efficient transfection relative to the left-most box.

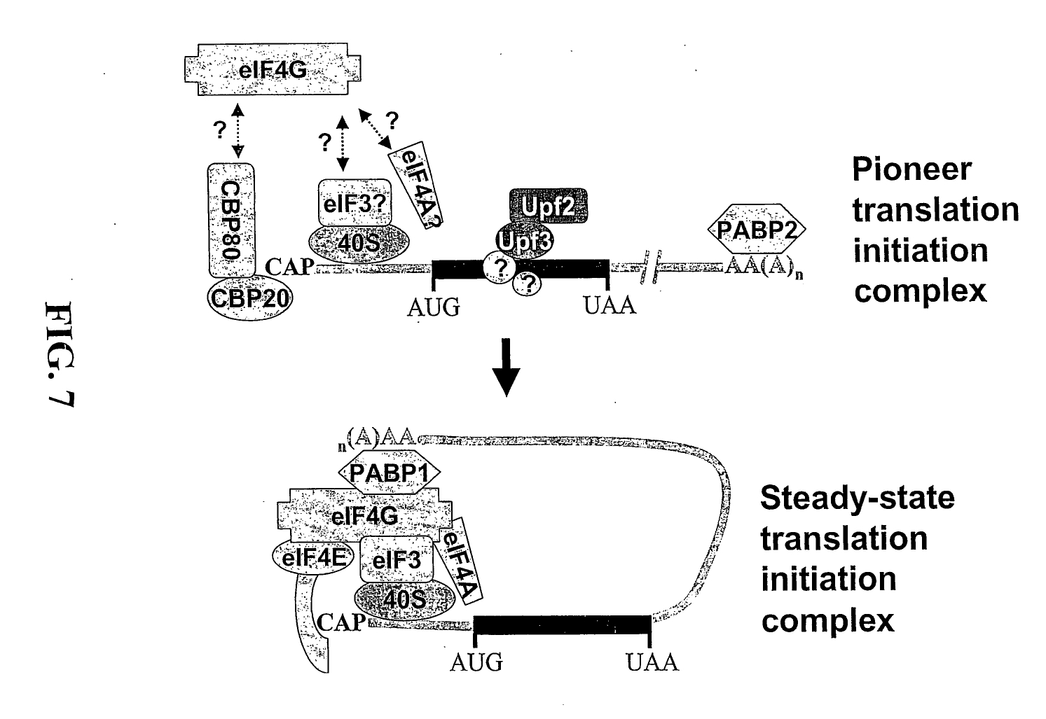

13. FIG. 7 shows a model for the pioneer translation initiation complex; shown relative to -the steady-state translation initiation complex. The pioneer translation initiation complex, which is the substrate for NMD (as well as the first round of translation for both nonsense-free and nonsnese-containing mRNAs), contains CBP80-CBP20 bound to the mRNA cap structure, PABP2 bound to the poly(A) tail, and Upf2 and Upf3 proteins probably bound close to, but upstream of, exon-exon junctions. Notably, the number of Upf2 and Upf3 molecules per cell is adequate for binding to the majority of exon-exon junctions. At least for mRNAs subject to cytoplasmic NMD, this complex functions in association with cytoplasmic ribosomes. It is uncertain if eIF4G is a multipurpose adaptor as it is in the steady-state translation initiation complex (Hentze, 1997; Gingras et al., 1999; Sachs and Varani, 2000), although it is present. Yellow and dark orange representations of proteins with questions marks signify the possible presence of components of the complex that binds mRNA as a consequence of splicing in the nucleus (Le Hir et al., 2000a&b) and is thought to recruit Upf3 protein. Alternatively, or additionally, Upf3 protein can interact with RNA directly since it contains an RRM-like domain (Lykke-Andersen et al., 2000). Upf1 protein is thought to associate after translation initiation, possibly as a consequence of translation termination. The presence of eIF3 has yet to be tested, and the low specificity of available anti-eIF4A antibody precluded eIF4A detection. The bulk of cellular translation involves the steady-state translation initiation complex, which derives from remodeling of the pioneer translation initiation complex.

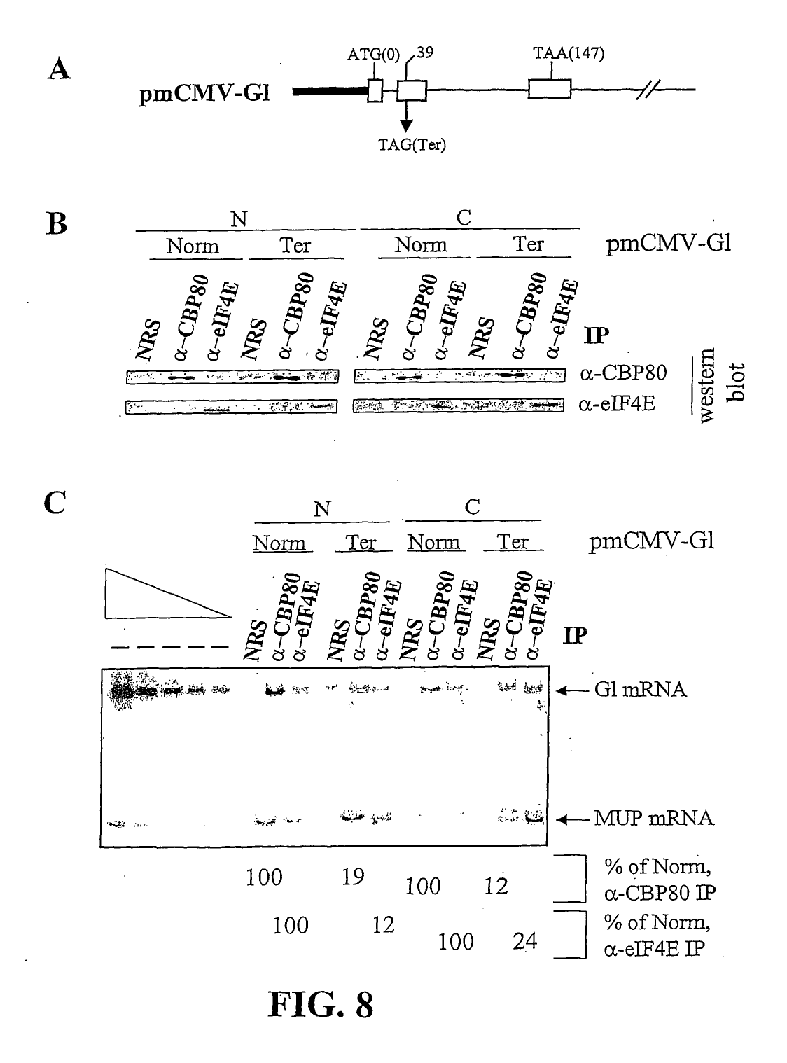

14. FIG. 8 shows that CBP80 and eIF4E are bound to mRNA in both nuclear and cytoplasmic cell fractions. (A) Structure of the pmCMV-Gl test plasmids, which harbor either a nonsense-free Gl allele that terminates translation at codon 147 (Norm) or a Gl allele carrying a TAG nonsense codon at position 39 (Ter). (3) Cos cells were transiently transfected with a test pmCMV-Gl plasmid and the reference plasmid phCMV-MUP. Nuclear (N) and cytoplasmic (C) fractions were then immunopurified under conditions that preserved RNP using either normal rabbit serum (NRS), anti-CBP80 antibody (α-CBP80), or anti-eIF4E antibody and subjected to western blotting using anti-CBP80 antibody or anti-eIF4E antibody. Nuclear fractions were free of cytoplasm as evidenced by the absence of detectable reactivity with either anti-eIF4A antibody or anti-PLCγ. (C) The levels of Gl and MUP mRNAs were quantitated in RNA prepared from each immunopurification using RT-PCR. The left-most five lanes, which analyze decreasing amounts of RNA before immunopurification (-IP), demonstrate that the conditions of RT-PCR were quantitative. Numbers below the Figure represent the level of Gl mRNA normalized to the level of MUP mRNA, where normalized levels of nonsense-free (Norm) mRNA in each anti-CBP80 and anti-eIF4E immunopurification of each fraction are defined as 100%. Results are representative of two independently performed experiments where the efficiency of NMD varied by no more than 12% (see text for specific standard deviations). The immunopurification using anti-eIF4E and cytoplasmic sample was less efficient for the transfection using pmCMV-Gl Norm than for the transfection using pmCMV-Gl Ter, explaining the reduced levels of eIF4E detected by western blotting (B) and RNA detected by RT-PCR (C).

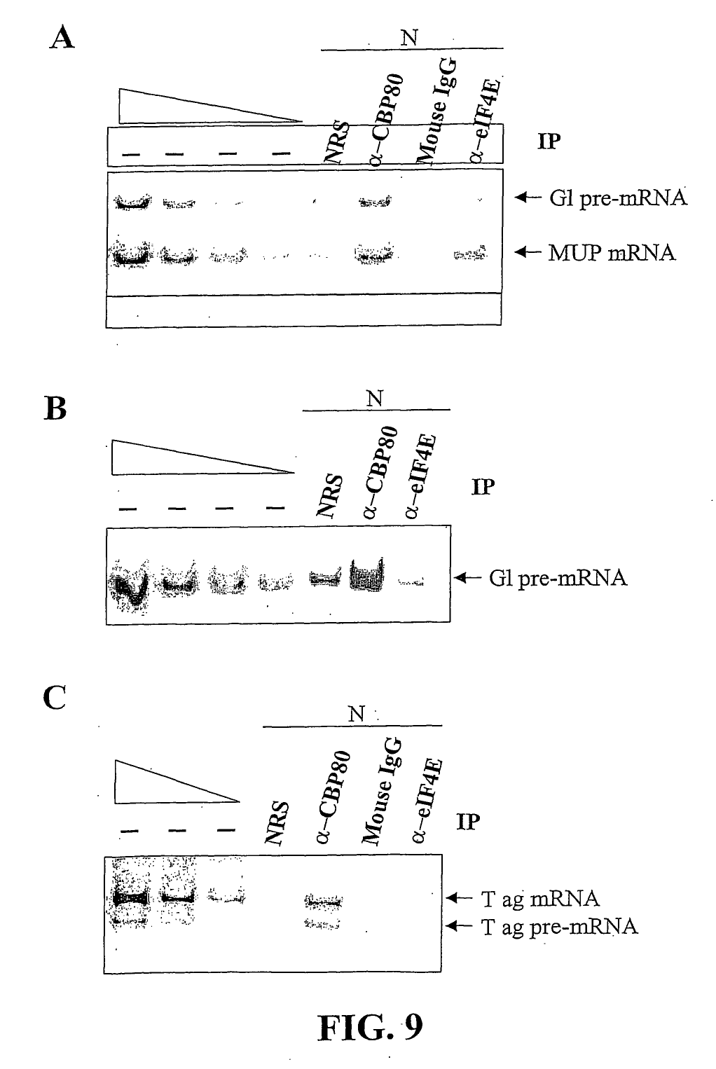

15. FIG. 9 shows that CBP80 but not eIF4E is detectably bound to intron-containing pre-mRNA. (A) As in FIG. 8 except that only nuclear fractions and Gl pre-mRNA rather than Gl mRNA were analyzed. Furthermore, mouse anti-eIF4E antibody was used instead of rabbit anti-eIF4E antibody and mouse IgG served to control for non-specific immunopurification using mouse anti-eIF4E. (B) As in panel (A) except that rabbit anti-eIF4E antibody was used in place of mouse anti-eIF4E antibody and the analysis of MUP mRNA was omitted. (C) As in panel (A) except that SV40 pre-mRNA and mRNA were analyzed. Results are representative of other independently performed experiments, including those using rabbit anti-eIF4E antibody and NRS as a control. Taking the sum of Gl or SV40 pre-mRNA immunopurified by anti-CBP80 antibody and anti-eIF4E antibody in each panel as 100%, the average amount of CBP80-bound pre-mRNA was 96% ∀ 6% and the average amount of eIF4E-bound pre-mRNA was 4 ∀ 6%.

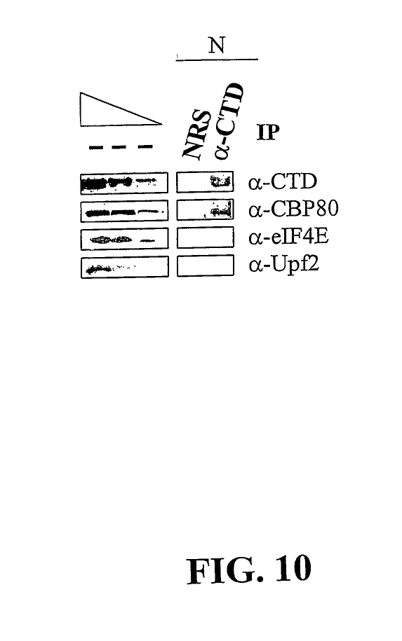

16. FIG. 10 shows that CBP80 but neither eIF4E nor Upf2 inmunopurifies with the CTD. The nuclear fraction of 32×10⁶ untransfected Cos cells was immunopurified using either NRS or anti-C-terminal domain of RNA polymerase II antibody (α-CTD), and protein from either 8×10⁵ cells (CTD) or 32×10⁵ cells (CBP80, eIF4E, Upf2) was subjected to western blotting using anti-CTD, anti-CBP80, anti-eIF4E or anti-Upf2 antibody. The three left-most lanes, which analyze two-fold dilutions of nuclear protein from 1.6×10⁵ cells (CTD, CBP80) or 6.4×10⁵ cells (eIF4E, Upf2) before immunopurification (-IP), demonstrate that the conditions of western blotting were semi-quantitative. The two forms of CTD likely differ in the degree of phosphorylation (Dubois et al., 1994). Results are representative of two-to-four independently performed experiments, depending on the antibody used in western blotting.

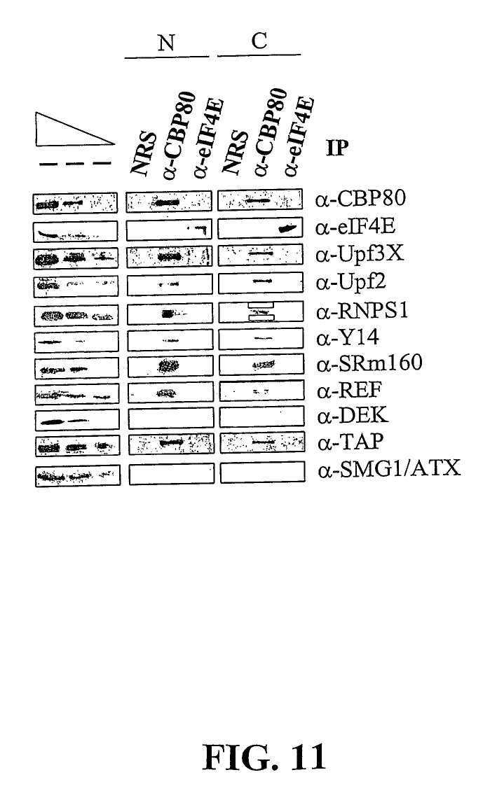

17. FIG. 11 shows that nuclear and cytoplasmic CBP80, unlike nuclear and cytoplasmic eIF4E, co-immunopurify with RNPS1, Y14, SRm160, REF/Aly, TAP, Upf3X and Upf2. Nuclear and cytoplasmic fractions of 32×10⁶ untransfected Cos cells were immunopurified using anti-CBP80 antibody, anti-eIF4E antibody or, as a control, NRS. Immunopurified protein from 8×10⁵ or 32×10⁵ cells was then analyzed by western blotting using the antibodies specified. The three left-most lanes, which analyze two-fold dilutions of nuclear protein from 8×10⁵ or 32×10⁵ cells before immunopurification (-IP), demonstrate that the conditions of western blotting were semi-quantitative. Notably, Upf3 likely co-migrates with the heavy chain of anti-hUpf3/3X antibody.

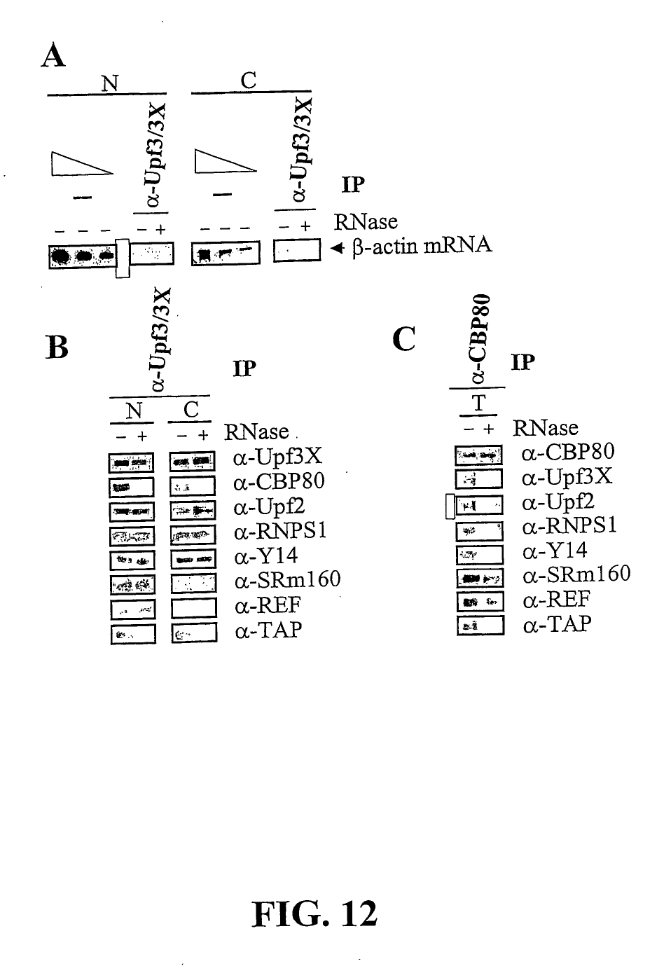

18. FIG. 12 shows RNPS1, Y14, SRm160, REF/Aly and Upf2 co-immunopurify with Upf3X in an RNase-insensitive manner, while RNPS1, Y14, REF/Aly, TAP, Upf3X and Upf2 co-immunopurify with CBP80 in an RNase-sensitive manner. Same as FIG. 4 except that an additional immunopurification using anti-Upf3/3X was performed, total (T) lysate was immunopurified using anti-CBP80 antibody, RNase was added to half of each sample prior to immunopurification, RNA as well as protein were prepared, and western blot analyses of eIF4E and DEK were omitted. (A) RT-PCR quantitation of the level of β-actin mRNA before or after immunopurification using anti-Upf3/3X antibody in order to demonstrate the efficiency of RNase treatment. Prior to immununopurification, half of the sample was treated with RNase (+), and the other half was not treated with RNase (−). (B) Western blot analysis of protein immunopurified using anti-Upf3/3X antibody from nuclear and cytoplasmic fractions. (C) Western blot analysis of protein immunopurified using anti-CBP80 antibody from total-cell extract.

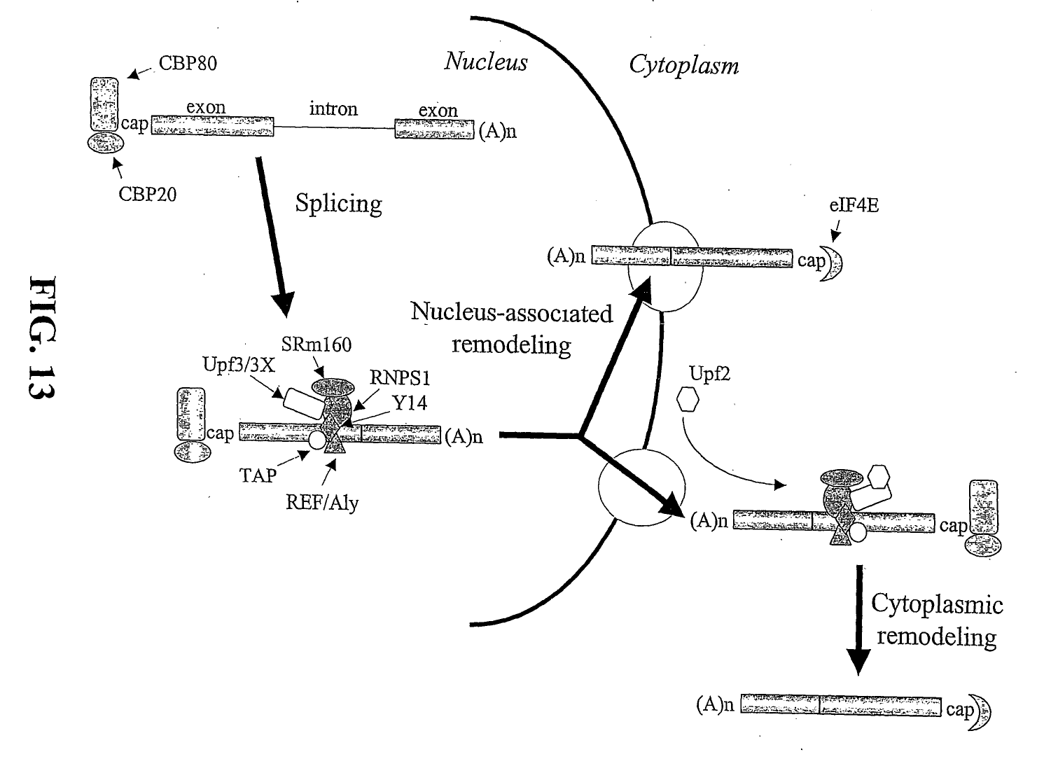

19. FIG. 13 shows a model for the dynamics of mRNP structure in mammalian cells as a consequence of pre-mRNA capping, pre-mRNA splicing and mRNA export. In the nucleus, CBP80-bound pre-mRNA is spliced to generate CBP80-bound mRNA. This mRNA is bound by an EJC (dark grey shapes), consisting of RNPS1, Y14, SRm160, and REF/Aly, located 20-24 nt upstream of the exon-exon junction. The EJC recruits the NMD factor Upf3/3X and the mRNA export factor TAP. Some mRNPs (possibly those subject to nucleus-associated NMD) undergo remodeling while nucleus-associated (nucleus-associated remodeling). Other mRNPs (including those subject to cytoplasmic NMD) undergo remodeling after export to the cytoplasm (cytoplasmic remodeling). In both types of remodeling, CBP80 is replaced by eIF4E and the EJC is lost. Data indicate that the mostly perinuclear Upf2 protein is recruited by the EJC at a point when mRNA co-purifies with nuclei. In theory, recruitment could occur during mRNA export. However, if nucleus-associated remodeling takes place in the nucleoplasm, it is conceivable that an as yet undetected pool of Upf2 is nucleoplasmic. The single exon-exon junction exemplified is likely to typify all splicing-generated exon-exon junctions, regardless of position relative to the mRNA 3′ end.

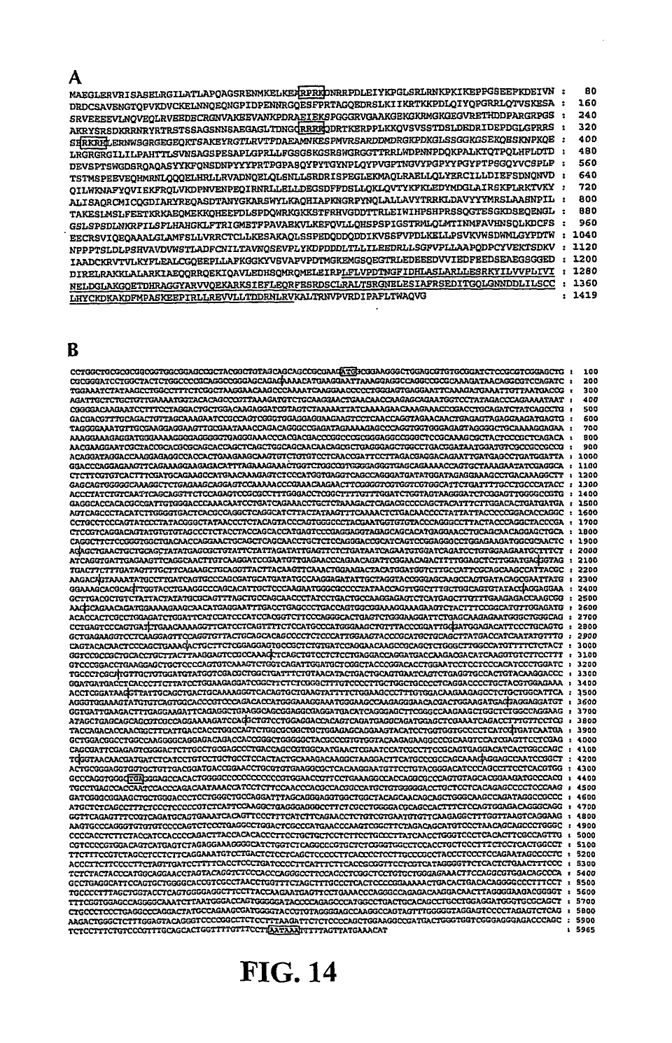

20. FIG. 14 shows hSmg5/7a amino acid (SEQ ID NO: 37) and hSMG5/7a cDNA (SEQ ID NO: 38) sequences. FIG. 14(A) shows the 1419-aa sequence of hSmg5/7a is numbered to the right. Underlined amino acids constitute regions of a putative PIN domain. Three predicted nuclear localization sequences are boxed. FIG. 14(B) shows the 5965-nt sequence of hSMG5/7a cDNA (KIAA0732) is numbered to the right. The ATG initiation codon, TGA termination codon, and AATAAA polyadenylation sequence are boxed; exon-exon junctions are labeled with bold vertical lines; and the 5′- and 3′-untranslated regions may be incomplete.

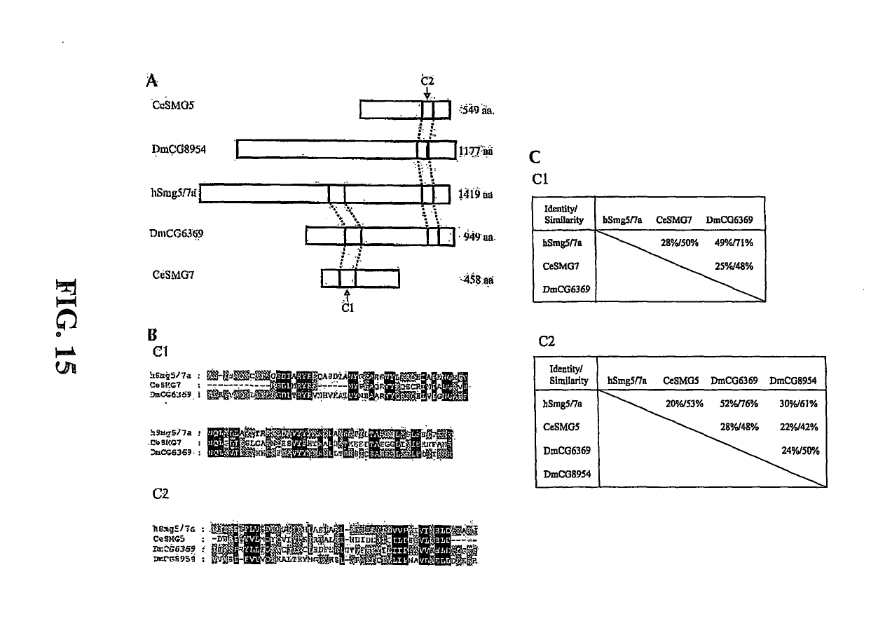

21. FIG. 15 shows an alignment of human Smg5/7a to Caenorhabditis elegans SMG5 and SMG7 and Drosophila melanogaster CG8954 and CG6369. FIG. 15(A) shows hSmg5/7a, C. elegans (Ce) SMG5, CeSMG7, and D. melanogaster (Dm) CG8954, and DmCG6369 are diagrammed as horizontal bars. The number of constituent amino acids (aa) is provided to the right of each bar. Conserved regions 1 (C1) and 2(C2 ) are boxed and aligned with dashes. FIG. 15(B) shows amino acids 721-816 of hSmg5/7a, 136-214 of CeSMG7, and 279-375 of DmCG6369 are aligned at C1; amino acids 1241-1276 of hSmg5/7a, 422-464 of CeSMG5, 765-815 of DmCG6369, and 1008-1055 of DmCG8954 are aligned at C2. White letters in black boxes specify amino acids that are either identical or similar among all compared proteins. White letters in gray boxes specify amino acids that are either identical or similar among only two (C1) or three (C2) compared proteins. Black letters in gray boxes specify amino acids that are either identical or similar between only two (C2) compared proteins. FIG. 15(C) shows the percent (%) identity/similarity between proteins within C1 or C2. Notably, considering the recently described CeSMG6 sequence, Smg5/7a is probably best renamed human Smg6, and Smg5/7b and c are best renamed human Smg5 and human Smg7, respectively (Gatfield et al., 2003; see below).

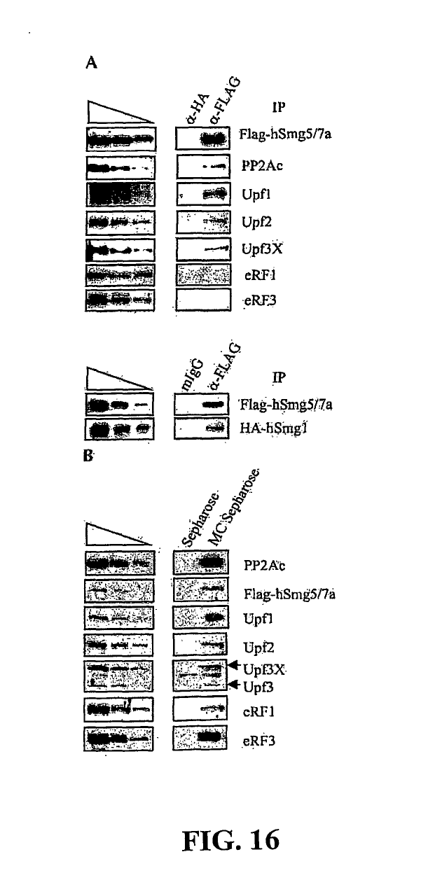

22. FIG. 16 shows that Flag-hSmg5/7a associates with PP2Ac, Upf1, Upf2, Upf3X, and hSmg1, but not with eRF1 and eRF3. FIG. 16(A) Flag-hSmg5/7a and, when specified, HA-hSmg1 were transiently expressed in Cos cells, and total-cell lysate was purified by using anti-Flag antibody, which purified 26% (top) or 3% (bottom) of cellular FlaghSmg5/7a, or anti-HA or mouse IgG, each of which controlled for nonspecific immunopurification. FIG. 16(B) should result with lysate that was purified by using microcystin (MC)-Sepharose, which purified 50% of cellular PP2Ac, or unconjugated Sepharose, which controlled for nonspecific binding to Sepharose alone. Purified material was then analyzed by Western blotting with the specified antibodies, which consisted of those against PP2Ac, Upf1, Upf2, Upf3/3X, eRF3, eRF1, Flag (to detect Flag-hSmg5/7a), and HA (to detect HA-hSmg1).

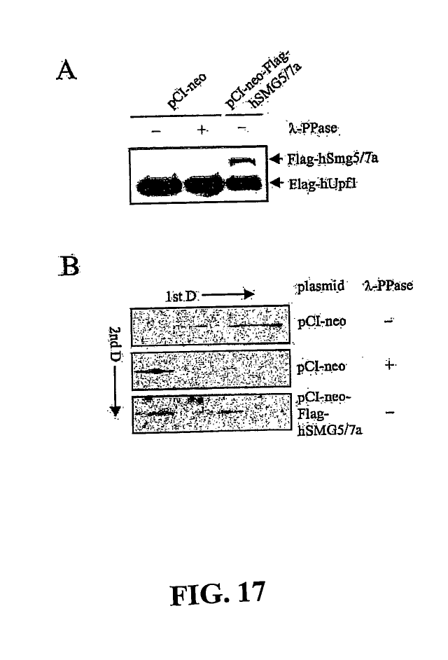

23. FIG. 17 shows that the overexpression of hSmg5/7a reduces the level of Upf1 phosphorylation. HEK293T cells were transiently transfected with pCI-neo-Flag-hUPF1 and either pCI-neo-Flag-hSMG5/7a or, as a control, pCI-neo. Total-cell lysates were purified by using anti-Flag antibody and incubated with or without λ-phosphatase (λ-PPase). FIG. 17(A) shows a fraction of each lysate prior to immunopurification was analyzed by Western blotting and anti-Flag antibody in order to quantitate the expression of Flag-hSmg5/7a and Flag-hUpf1 in each sample. FIG. 17(B) Comparable amounts of immunopurified FlaghUpf1 were then subjected to two-dimensional gel electrophoresis under conditions that precluded detection of Flag-hSmg5/7a, and Flag-hUpf1 was localized by blotting with anti-Flag antibody.

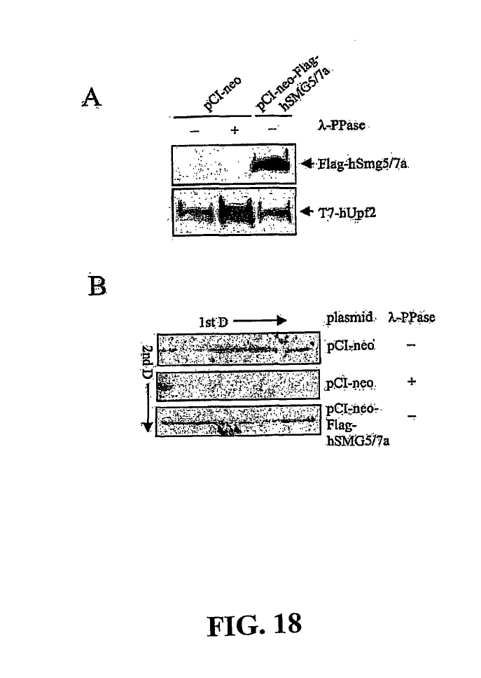

24. FIG. 18 shows hUpf2is a phosphoprotein, but overexpression of hSmg5/7a does not reduce the level of hUpf2phosphorylation. HEK293T cells were transiently transfected with pCI-neo-T7-hUPF2 and either pCI-neo-Flag-hSMG5/7a or, as a control, pCIneo. Total-cell lysates were purified by using anti-T7 antibody and incubated with or without λ-phosphatase (λ-PPase). FIG. 18(A) shows a fraction of each lysate was analyzed by Western blotting and anti-T7 or anti-Flag antibody in order to demonstrate the presence of T7-hUpf2and Flag-hSmg5/7a. FIG. 18(B) shows comparable amounts of immunopurified T7-hUpf2were then subjected to two-dimensional gel electrophoresis, and T7-hUpf2was localized by Western blotting and anti-T7 antibody.

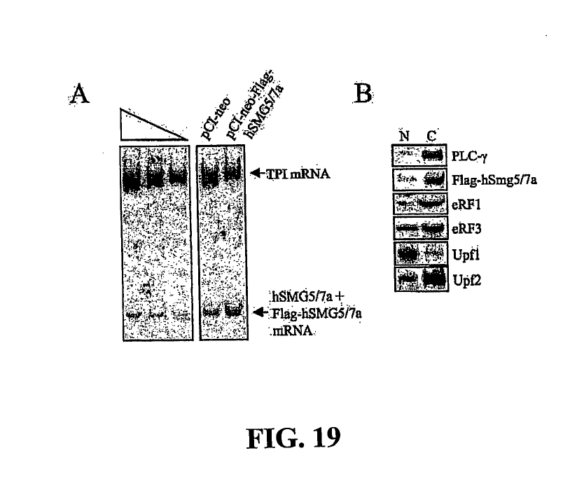

25. FIG. 19 shows that hSmg5/7a localizes primarily to the cytoplasmic fraction of HEK293T cells. Flag-hSMG5/7a was transiently expressed in HEK293T cells. Subsequently, RNA was purified from total-cell lysates, and protein was purified from nuclear and cytoplasmic fractions. FIG. 19(A) shows RT-PCR was used to determine the level at which Flag-hSmg5/7a was expressed relative to endogenous Smg5/7a. Considering a transfection efficiency of 90%, the combined levels of endogenous and exogenous hSMG5/7a mRNA in transfected cells was determined to be only 2.6-fold of the level of endogenous hSMG5/7a mRNA in untransfected cells. FIG. 19(B) shows Western blotting using antibodies against PLC-γ, Flag (to detect Flag-hSmg5/7a), eRF1, eRF3, Upf1, and Upf2was used to determine the cellular distribution of the various proteins.

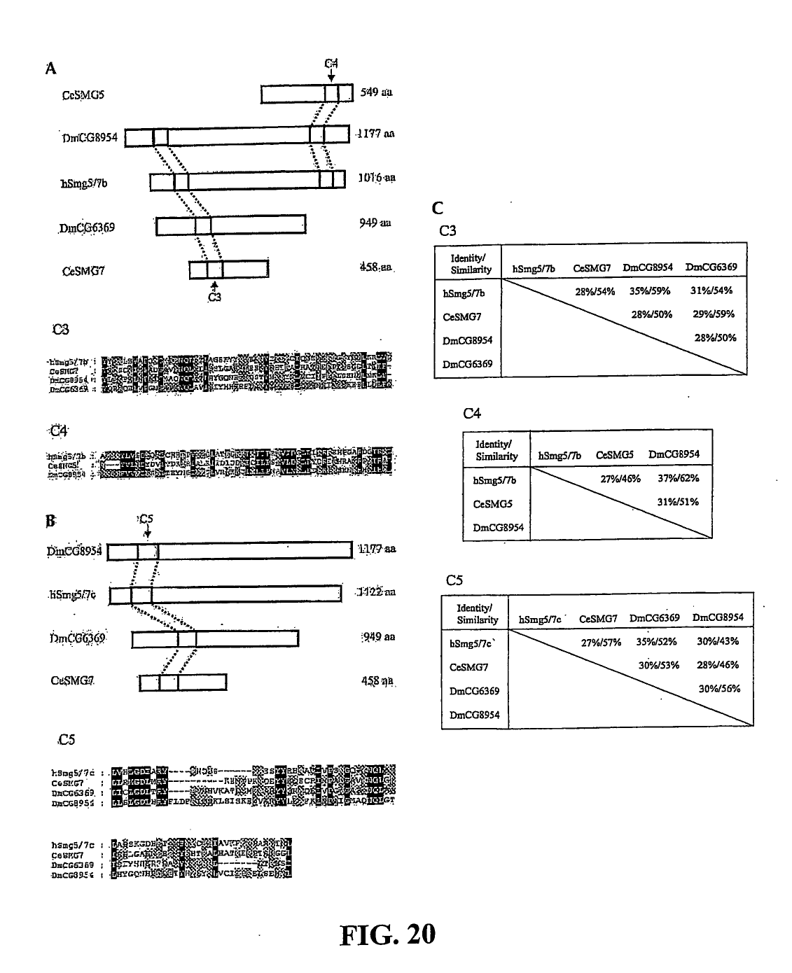

26. FIG. 20 shows the alignment of hSmg5/7b and hSmg5/7c to CeSMG5 and CeSMG7 and DmCG8954, and DmCG6369. FIG. 20(A, B) shows hSmg5/7b or hSmg5/7c, CeSMG5, CeSMG7, DmCG8954, and DmCG6369 are diagrammed as horizontal bars. The number of constituent amino acids (aa) is provided to the right of each bar. Conserved regions 3 (C3), 4 (C4), and 5 (C5) are boxed and aligned with dashes. Amino acids 173-257 of hSmg5/7b, 130-208 of CeSMG7, 286-370 of DmCG6369, and 211-298 of DmCG8954 can also be aligned at C3. Amino acids 853-924 of hSmg5/7b, 1009-1081 of DmCG8954, and 425-480 of CeSMG5 can be aligned at C4. Amino acids 186-257 of hSmg5/7c can be aligned with amino acids 133-208 of CeSMG7, 288-361 of DmCG6369, and 213-299 of DmCG8954 at C5. White letters in black boxes, white letters in gray boxes, and black letters in gray boxes are as in FIG. 14B. FIG. 20(C) shows the percent (%) identity/similarity between proteins within C3, C4, and C5. As noted above, considering the recently described CeSMG6 sequence, Smg5Ma is probably best renamed human Smg6, and Smg5/7b and Smg5/7c are best renamed human Smg5 and human Smg7, respectively (Gatfield et al., 2003).

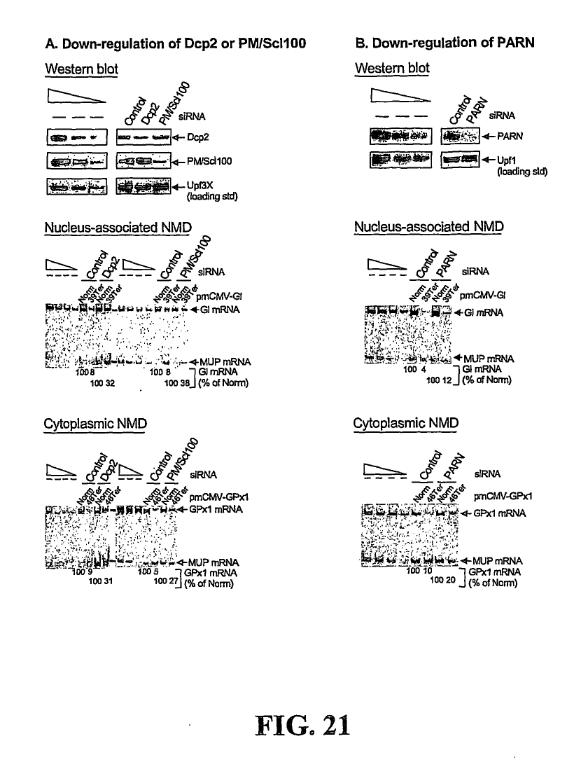

27. FIG. 21 shows SiRNA-mediated down-regulation of the Dcp2, PM/Scl100 or PARN increases the level of nonsense-containing Gl mRNA, which is subject to nucleus-associated NMD, as well as the level of nonsense-containing GPx1 mRNA, which is subject to cytoplasmic NMD. FIG. 21(A) shows that HeLa cells (2×10⁶) were transiently transfected with 180 ng of a polIII promoter-containing PCR fragment that directs the synthesis of Dcp2 siRNA in vivo, or 600 pmol of in vitro-synthesized PM/Scl100 siRNA or a non-specific “Control” siRNA. Cells were re-transfected four days later with the same PCR fragment or siRNA, 3 μg of a pmCMV-Gl test plasmid and 3 μg of a GPx1 test plasmid, either nonsense-free (Norm) or nonsense-containing (39Ter or 46Ter, respectively), and 1 μg of the reference phCMV-MUP plasmid. Cells were harvested after an additional two days. Protein was purified from half of the cells, and RNA was purified from nuclear and cytoplasmic fractions of the remaining cells. (A, Upper) Western blot analysis of total-cell protein (20 μg) demonstrates that siRNA down-regulated the level of Dcp2 or PM/Scl100 to ˜35% of normal when Upf3X was used to control for differences among lanes in protein loading. The three or four left-most lanes, which consist of 2-fold dilutions of cellular protein, indicate that the analysis is semi-quantitative. FIG. 21(A, Middle) shows RT-PCR analysis of nucleus-associated Gl and MUP mRNAs. The left-most four lanes, which analyze decreasing amounts of RNA, demonstrate that the conditions of RT-PCR were quantitative. Numbers below the Figure represent the level of Gl mRNA normalized to the level of MUP mRNA, where the normalized level of nonsense-free (Norm) mRNA in the presence of each siRNA is defined as 100%. FIG. 21(A, Lower) shows RT-PCR analysis of cytoplasmic GPx1 and MUP mRNAs, essentially as described in (A, Middle). FIG. 21(B) shows that HeLa cells (2×10⁶) were transiently transfected with 600 ng of a polIII promoter-containing PCR fragment that directs the synthesis of PARN siRNA in vivo or a non-specific “Control” siRNA. FIG. 21 (B, Upper) as in (A, Upper), except that the level of PARN was shown to be down-regulated by siRNA to ˜15% of normal. FIG. 21(B, Middle and Lower) as in (A, Middle and Upper). All results are representative of at least three independently performed experiments.

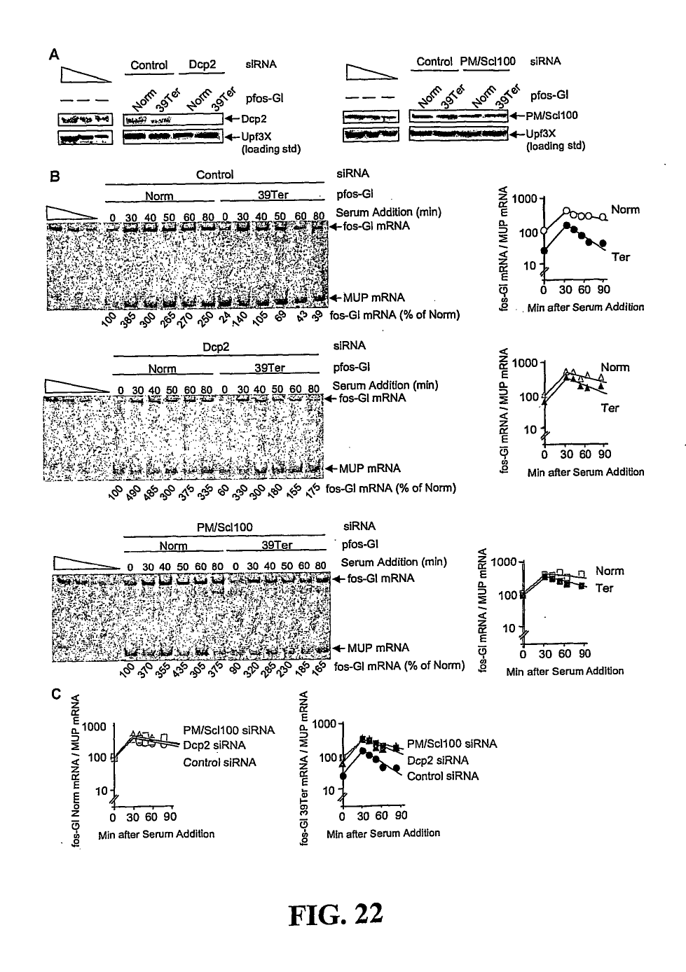

28. FIG. 22 shows SiRNA-mediated down-regulation of Dcp2 or PM/Scl100 decreases the rate at which fos-Gl 39Ter mRNA undergoes NMD. L cells (4×10⁶) were transiently transfected as described in the legend to FIG. 1. However, the test plasmid was either pfos-Gl Norm or pfos-Gl 39Ter. Furthermore, serum was removed 12 hr after the second transfection. After an additional 24 hr, serum was added to 15%. Protein was purified from the cytoplasmic fraction and RNA was prepared from the nuclear fraction at the specified times thereafter. FIG. 22(A) shows Western blot analysis of cytoplasmic protein (10 μg) at 0 min after serum addition demonstrates that siRNA down-regulated the level of Dcp2 or PM/Scl100 to, respectively, <10% or ˜40% of normal. For each analysis, the level of Upf3X was used to control for differences between samples in protein loading. FIG. 22(B) shows RT-PCR analysis of levels of nucleus-associated fos-Gl and MUP mRNAs demonstrates that down-regulation of Dcp2 or PM/Scl100 increases the half-life of fos-Gl 39Ter mRNA. The three or four left-most lanes analyzing decreasing amounts of RNA demonstrate that the RT-PCR conditions were quantitative. For each time point, the level of fos-Gl mRNA was normalized to the level of MUP mRNA. Normalized levels were calculated as a percentage of the normalized levels of either fos-Gl Norm mRNA (for both Norm and Ter mRNAs) or fos-Gl 39Ter mRNA (for Ter mRNA) at time 0, which were defined as 100, and are specified below each RT-PCR analysis. To the right of each RT-PCR analysis, normalized levels are plotted as a function of time after serum addition. FIG. 22(C) shows a comparison of the relative rates of fos-Gl Norm mRNA (left) or fos-Gl 39Ter mRNA decay (right) in the presence of Control, Dcp2 or PM/Scl100 siRNA using data obtained in (B).

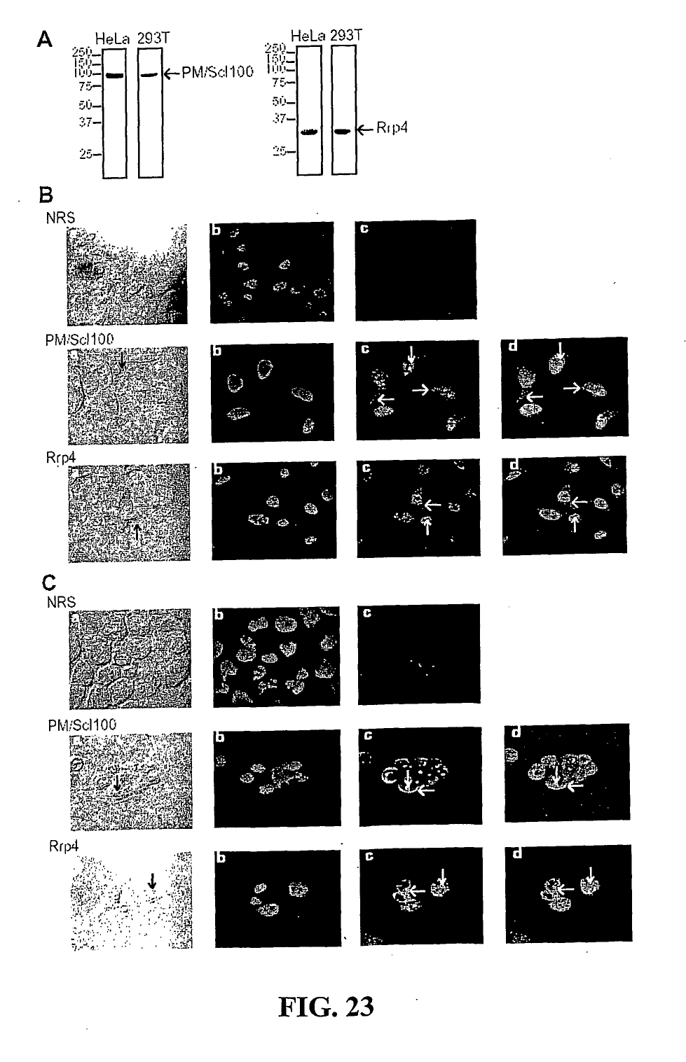

29. FIG. 23 shows that PM/Scl100 localizes to both nuclei and cytoplasm of HeLa and 293T cells using indirect immunofluorescence and confocal microscopy. FIG. 23(A) shows a Western blotting demonstrating that the immunoreactivity of HeLa-cell and 293T-cell extracts with each antibody used for immunolocalization is specific to the protein of interest. The molecular weights of standards are shown to the left of each Western blot. FIG. 23(B) shows the immunolocalization of PM/Scl100 and, as a control, Rrp4 in HeLa cells. For each antibody, panels represent the following: (a) differential interference contrast, (b) staining of nuclei by DAPI, (c) immunofluorescence of each antibody, and (d) overlay of panels (b) and (c). Vertical arrows specify exemplary nucleoli, and horizontal arrows specify exemplary cytoplasm. NRS, normal rabbit serum. FIG. 23(C) same as (B), only 293T cells were analyzed.

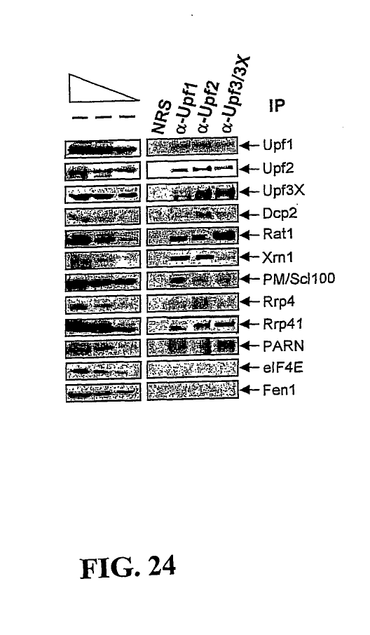

30. FIG. 24 shows Upf1, Upf2 and Upf3X co-immunopurify with components of the 5′-to-3′ and 3′-to-5′ mRNA decay pathways. Proteins and RNP from 3×10⁷ untransfected Cos cells were subjected to immunopurification (IP) using total-cell extract and anti-Upf1, anti-Upf2, or anti-Upf3/3X antibody or, as a control for non-specific IP, normal rabbit serum (NRS). Proteins present in each IP were then analyzed by Western blotting using the antibody specified to the right of each panel. The three left-most lanes analyzed decreasing amounts of protein before IP and demonstrate that the conditions used for Western blotting are semi-quantitative. IP efficiencies were 10% for Upf1, 10% for Upf2 and 15% for Upf3X. Notably, the co-migration of Upf3 with antibody heavy chain precludes its detection. Results are representative of three independently performed experiments in that all degradative proteins that were assayed were invariably detected in all IPs, albeit with efficiencies that sometimes varied between IPs.

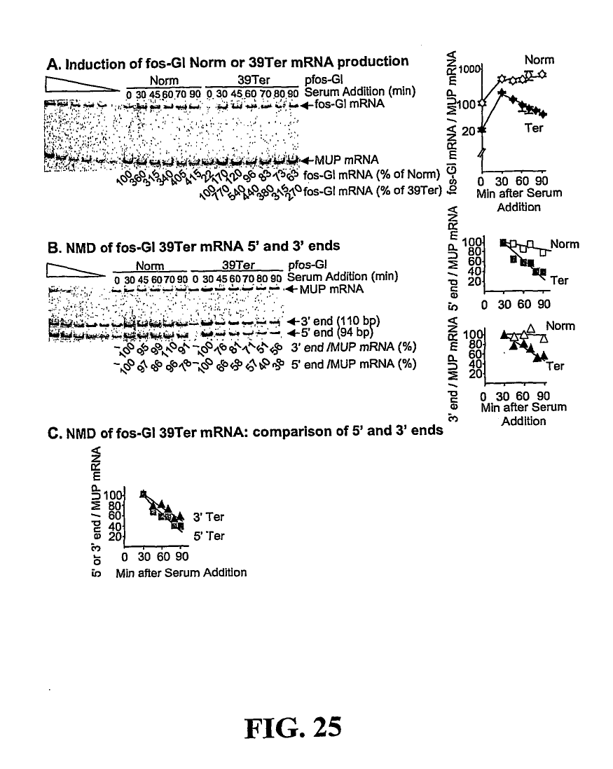

31. FIG. 25 shows the 5′ end of transiently induced fos-Gl 39Ter RNA is degraded at a rate that is comparable to the decay rate of full-length mRNA and at a comparable for slightly faster than the decay rate of the 3′ end. FIG. 25(A) shows the NMD of serum-induced fos-Gl 39Ter mRNA is evident after 30 min of serum addition to serum-deprived L-M(TK) cells. L cells were transiently transfected and analyzed as described in the legend to FIG. 22 except siRNA was omitted. Error bars specify the extent of deviation among three independently performed experiments. FIG. 25(B) shows RT-PCR analysis of the relative rates of fos-Gl Norm and 39Ter mRNA 5′-end and 3′-end decay. As in (A), however the levels of fos-Gl mRNA 5′ and 3′ ends were analyzed as 94-bp and 110-bp RT-PCR products, respectively, and data were plotted on a linear scale. FIG. 25(C) shows a comparison of the relative rates of fos-Gl 39Ter 5′-end and 3′-end decay using data obtained in (B).

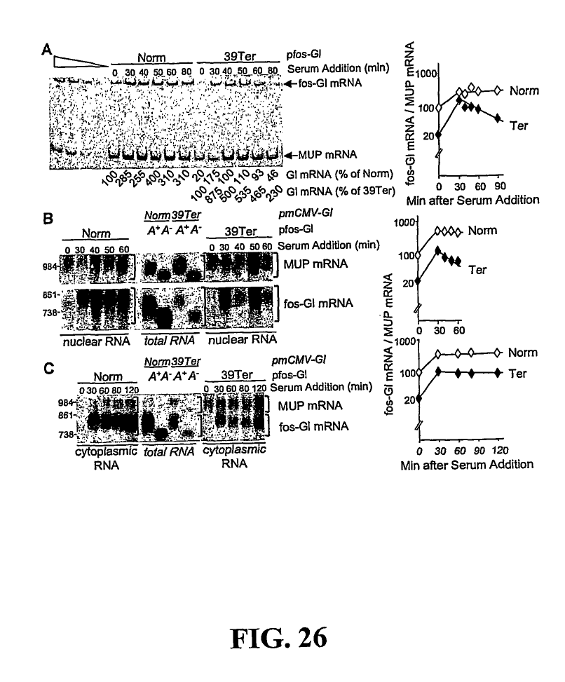

32. FIG. 26 shows the NMD of fos-Gl 39Ter mRNA is not accompanied by detectable deadenylated intermediates. L cells were transfected as in FIG. 25. However, cells were harvested at different times after serum addition, and both nuclear and cytoplasmic RNA was prepared. FIG. 26(A) shows that nuclear RNA was analyzed by RT-PCR as described in FIG. 5A. FIG. 26(B) shows that nuclear RNA (4 μg) was analyzed by Northern blotting. The lengths of the poly (A) tails on newly-synthesized fos-Gl Norm and Ter mRNAs were assessed relative to the lengths of the poly (A) tail on of steady-state Gl Norm and Ter mRNAs (i.e., Gl mRNA driven by the mouse CMV promoter) in 2 μg total-cell RNA before (A⁺) and after (A⁻) deadenylation in vitro using 0.2 μg-of oligo (dT) and 0.1 U of RNase H. A lighter Phosphorimager exposure is shown for lanes containing pmCMV-Gl “mRNA (total RNA) relative to fos-Gl Norm and Ter mRNAs (nuclear RNA) even though all RNAs were analyzed in the same gel. MW standards are shown to the left. FIG. 26(C) shows cytoplasmic RNA (4 μg) that was analyzed as in (B).

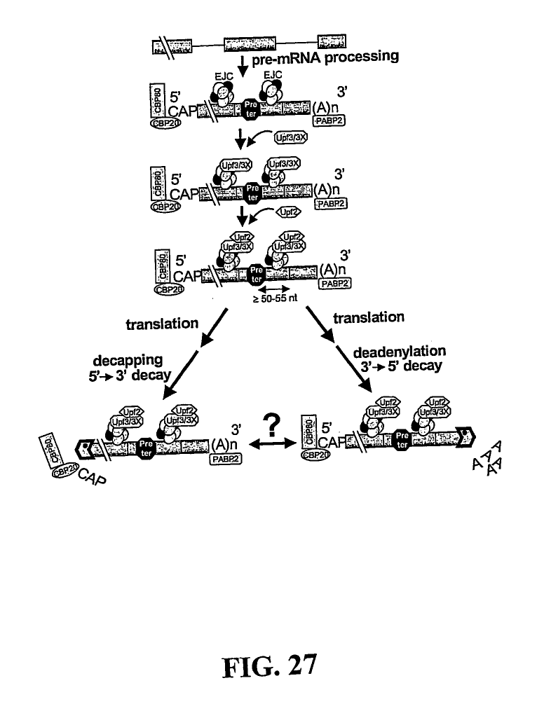

33. FIG. 27 shows the model for the polarity and enzymology of NMD in mammalian cells. NMD takes place during what is called the “pioneer” round of translation (Ishigaki et al., 2001; Lejeune et al., 2002). The pioneer translation initiation complex involves mRNA bound by CBP80/20 at the 5′ cap, PABP2 at the 3′ poly(A) tail, and an exon junction complex (EJC) that is deposited as a consequence of pre-mRNA splicing at each exon-exon junction. The EJC recruits Upf3 or 3X, each of which is a mostly nuclear shuttling protein involved in NMD (Lykke-Andersen et al., 2000; Serin et al., 2001). Upf3/3X then recruits Upf2, another protein involved in NMD that appears concentrated on the cytoplasmic side of the nuclear envelope (Lykke-Andersen et al., 2000; Serin et al., 2001). Upf1, a group 1 RNA helicase that is mostly cytoplasmic but also nuclear (Mendell et al., 2002), is not detected as an integral mRNP protein but presumably triggers NMD by interacting with EJC-bound Upf2 when translation terminates at a premature termination codon (Pre Ter) that is located more than 50-55 nucleotides upstream of the corresponding exon-exon junction. Data indicate that NID initiates from both the mRNA 5′ end so as to involve decapping followed by 5′-to-3′ exonucleolytic decay and the mRNA 3′ end so as to involve deadenylation followed by exosome-mediated 3′-to-5′ exonucleolytic decay, which can take place on the same molecule. It is currently not possible to determine the relative efficiencies of 5′-to-3′ and 3′-to-5′ decay during NMD.

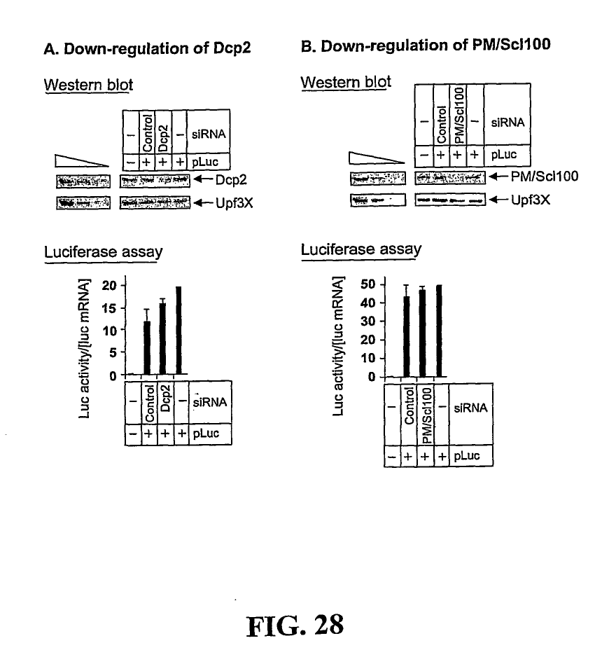

34. FIG. 28 shows the down-regulation of either Dcp2 or PM/Scl100 protein does not down-regulate cellular translation as measured by renilla luciferase activity. HeLa cells (2×10⁶ cells) were transiently transfected with 180 ng of a pol III promoter-containing PCR fragment that directs the synthesis of Dcp2 siRNA in vivo, or 600 pmol of in vitro-synthesized PM/Scl100 siRNA, a non-specific Control siRNA, or no siRNA (−) as described in the legend to FIG. 1. Cells were re-transfected three days later with the same PCR fragment or siRNA or nothing and 0.5 μg of pRenilla Luciferase (pLuc) (obtained from Sung Key Jang; Kim et al., 2003) or empty vector (−). Cells were harvested after an additional two days. Protein was purified from half of the cells, and RNA was purified from the other half. (A and B, Upper). Western blot analysis of total-cell protein (10 μg) demonstrates that siRNA down-regulated the level of Dcp2 and PM/Scl100 to, respectively, 44% and <10% of normal when the level of Upf3X was used to control for differences among lanes in protein loading. The three left-most lanes, which consist of 2-fold dilutions of cellular protein, indicate that the analysis is semi-quantitative. (A and B, Lower). Renilla luciferase activity assay using total-cell protein demonstrates that down-regulating the level of either Dcp2 or PM/Scl100 does not significantly affect the amount of luciferase activity. For each sample, luciferase (Luc) activity was normalized to the level of luciferase mRNA in order to control for variations in transfection efficiencies. Error bars specify the extent of deviation between two independently performed experiments.

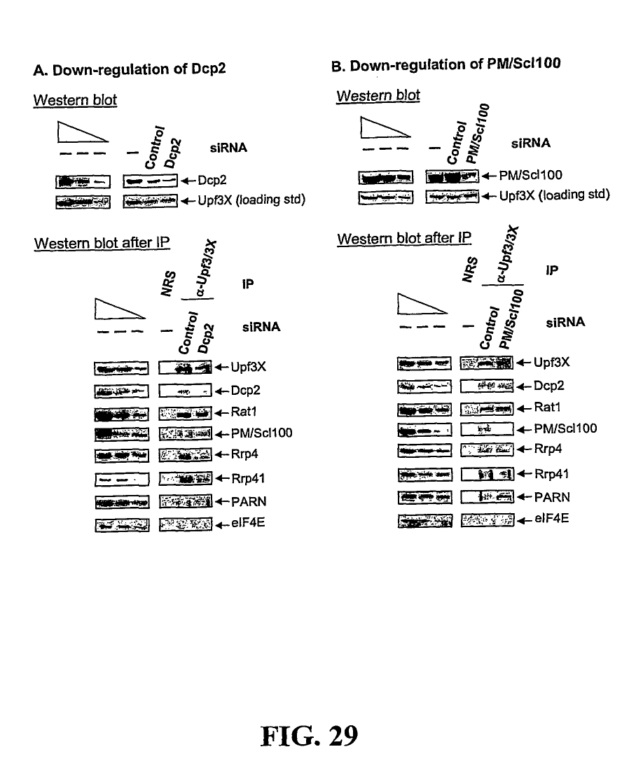

35. FIG. 29 shows that the down-regulation of Dcp2 or PM/Scl100 does not disrupt the co-immunopurification of Upf3X with components of the 5′-to-3′ and 3′-to-5′ mRNA decay pathways, ruling out the formal possibility that, e.g., down-regulation of PM/Scl100 abrogated NMD not because 3′-to-5′ decay is involved but because 5′-to-3′ decay was debilitated. HeLa cells (8×10⁶ cells) were transiently transfected with 720 ng of a pol III promoter-containing PCR fragment that directs the synthesis of Dcp2 siRNA in vivo or a non-specific Control siRNA. Alternatively, cells (8×10⁶) were transiently transfected with 2400 pmol of either in vitro-synthesized PM/Scl100 siRNA or a non-specific Control siRNA. Cells were re-transfected three days later with the same PCR fragment or siRNA and harvested after an additional two days. Total-cell protein was purified. The majority (9/10) was subjected to immunopurification (IP) under conditions that maintain mRNP using total-cell extract and either anti-Upf3/3X antibody or, as a control for non-specific IP, NRS. (A, B Upper) Western blot analysis of total-cell protein before IP (20 μg) demonstrates that siRNA down-regulated the level of Dcp2 or PM/Scl100 to ˜40% or <10% of normal, respectively, when Upf3X was used to control for differences among lanes in protein loading. The three left-most lanes, which consist of 2-fold dilutions of cellular protein, indicate that the conditions used for Western blotting are semi-quantitative. (A,B lower) Proteins present in each IP were analyzed by Western blotting using the antibody specified to the right of each panel. The three left-most lanes analyzed decreasing amounts of protein before IP and demonstrate that the Western blot is semi-quantitative. (A, lower) IP efficiencies in were 10% (Control siRNA) or 5% (D)cp 2 siRNA). Notably, given this difference in IP efficiencies, Western blot intensities indicate that the complex remains intact after Dcp2 down-regulation. (B, lower) IP efficiencies were 6% for both Control and PM/Scl100 siRNAs. As usual, the co-migration of Upf3 with antibody heavy chain precludes its detection.

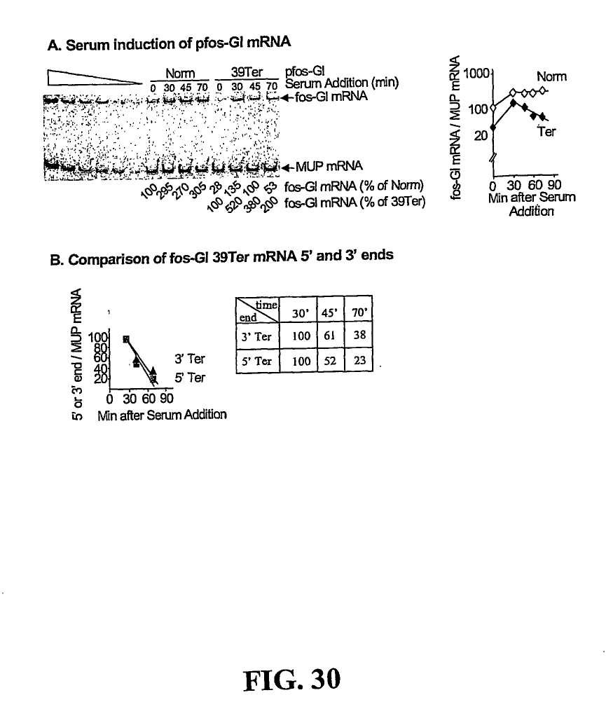

36. FIG. 30 shows independent analysis (i.e, independent of data shown in FIG. 25) of the rates at which the 5′ and 3′ ends of transiently induced fos-Gl 39Ter mRNA are degraded. FIG. 30(A) shows the NMD of serum-induced fos-Gl 39Ter mRNA is evident after 30 min of serum addition to serum-deprived L-M(TK⁻) cells. L cells were transiently transfected and analyzed as described in the legend to FIG. 5. FIG. 30(B) shows data derived from RT-PCR analysis demonstrating that the 5′ end of fos-Gl 39Ter mRNA is degraded slightly faster than the 3′ end. As in (A), however, the levels of fos-Gl 39Ter mRNA 5′ and 3′ ends were analyzed as 94-bp and 110-bp RT-PCR products, respectively, and data were plotted on a linear scale.

### IV. DETAILED DESCRIPTION

37. The present invention may be understood more readily by reference to the following detailed description of preferred embodiments of the invention and the Examples included therein and to the Figures and their previous and following description.

38. Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

39. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

40. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

41. In this specification and in the claims which follow, reference will be made to a number of terms which shall be defined to have the following meanings:

42. “Optional” or “optionally” means that the subsequently described event or circumstance may or may not occur, and that the description includes instances where said event or circumstance occurs and instances where it does not.

43. As used herein, “pioneer round of translation” or “pioneering round” refers to an initial round of mRNA translation prior to steady-state translation. The round is characterized by the presence of CBP80 and CBP20 as the CAP binding proteins, PABP2 as a polyadenylation binding protein and involves Upf2 and Upf3 or Upf3X.

44. As used herein, “chemical” refers to any reagent that can bind, mutate, dissolve, cleave, initiate a reaction that results in a new substance, or alter the confirmation of a target substance. An example of a chemical would be small molecules.

45. As used herin, “interacts” means to affect a substance either directly or indirectly through cooperative function, competitive inhibition, non-competitive inhibition, binding, or contacting the substance, a target molecule, an accessory molecule, or alternative portion of a system so as to effect at least one function. The interaction can be stimulatory or cooperative in nature having an additive or synergistic effect. The interaction can also result in the inhibition of a process or target molecule

46. As used herein, “system” refers to any cell, organism, or in vitro assay or culture. Such a system includes components necessary for NMD activity. Such components can include, for example, but are not limited to Upf1, Upf2, Upf3, Upf3X, PABP2, CBP20, CBP80, RNPS1, Y14, SRm160, REF, and TAP.

47. By “NMD complex” is any combination of one or more of the essential components of NMD in the pioneering round of translation.

48. As used herein, “subject” refers to any cell, tissue, or organism used to study or treat for a disorder relating to NMD. Including, for example, human patients with conditions that result from NMD or cell lines used to study aspects of NMD.

49. By “modulation” or “modulating” is meant either an increase or a decrease in NMD activity. Whether the NMD levels are increased or decreased in a subject with a condition resulting from a nonsense mutation depends on the particular sample. For example, with cystic fibrosis, a nonsense mutation results in NMD to remove the truncated RNA prior to translation. A decrease in NMD activity would increase the amount of mRNA from the truncated protein available for translation.

50. By “acquired disorder” is meant any disorder that lacks a clear genetically inherited link. For example, an infection or malignancy without a clear genetically inherited link.

### B. COMPOSITIONS AND METHODS

51. Disclosed are methods of treating a disorder in a subject comprising administering to the subject a substance, wherein the substance modulates (e.g., increases or decreases) nonsense-mediated mRNA decay in the pioneer round of translation.

52. Also disclosed are methods of the invention, wherein the disorder is an inherited genetic disorder. The genetic disorder can be selected from the group consisting of cystic fibrosis, hemophilia, Tay-Sachs disease, other mucopolysaccharidoses, Duchenne muscular dystrophy, other dystrophies, β°-thalassemia, other anemias, triose phosphate isomerase deficiency, other glycolytic enzyme deficiencies, Marfan Syndrome, other connective tissue disorders, ataxia telangiectasia, other DNA repair disorders, Alzheimer's disease, other dementias, Sandhoffdisease, epidermolysis bullosa simplex, insulin resistance, maple syrup urine disease, hereditary fructose intolerance, X-linked severe combined immunodeficiency, other immunodeficiencies, inherited cancers such as those due to BRCA-1 nonsense mutations, carbohydrate metabolism disorders, amino acid metabolism disorders, lipoprotein metabolism disorders, lipid metabolism disorders, lysomal enzymes metabolism disorders, steroid metabolism disorders, purine metabolism disorders, pyrimidine metabolism disorders, metal metabolism disorders, porphyrin metabolism disorders, or heme metabolism disorders.

53. Also disclosed are methods of the invention, wherein the disorder is an acquired disorder. The acquired disorder is, by way of example but not by way of a limitation, a cancer. Such a cancer may be related to mutations such as mutations in p53 or BRCA-1.

54. Also disclosed are methods of the invention, wherein the acquired disorder is a cancer. The cancer to be treated is selected from the group consisting of lymphoma, B cell lymphoma, T cell lymphoma, leukemia, carcinoma, sarcoma, glioma, blastoma, neuroblastoma, plasmacytoma, histiocytoma, melanoma, adenoma, mycosis fungoide, hypoxic tumor, myeloma, metastatic cancer, bladder cancer, brain cancer, nervous system cancer, head and neck cancer, ovarian cancer, pancreatic cancer, prostate cancer, skin cancer, liver cancer, colon cancer, cervical cancer, cervical carcinoma, breast cancer, epithelial cancer, renal cancer, genitourinary cancer, pulmonary cancer, esophageal carcinoma, hematopoietic cancers, testicular cancer, colorectal cancer, and prostatic cancer.

55. In one embodiment the subject is a mammal. It is specifically contemplated that mammal can include but is not limited to human, monkey, mouse, dog, pig, rabbit, and cow.

56. Also disclosed are methods of the invention, wherein the substance interacts with Upf1, Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, Rrp41 or a combination thereof. Deletion of amino acids 1095-1272 and, to a lesser extent, amino acids 94-133 of Upf2 inhibits interaction with Upf1 (Serin et al., 2001). Deletion of amino acids 711-928 of Upf2 inhibits interaction with Upf3, Upf3X, and a slicing variant of Upf3, called Upf3Δ (Serin et al., 2001). Mutation of either the Upf3X amino acids 5-58 (a putative NES) or amino acids 117-119 (YVF-to-DVD) or deletion of amino acids 30-255 inhibits interactio with Upf2 (Serin et al., 2001). Thus specific fragments of Upf2 or other proteins could be selected whereby the substance interacts with a selected fragment or portion of the protein.

57. The modulating substance used in the method is a functional nucleic acid, peptide, protein, antibody, or chemical.

58. Disclosed are methods of screening for a substance that modulates a nonsense-mediated mRNA decay (NMD) complex (optionally, during the pioneer round) comprising incubating the substance with the all or part of the complex, and assaying for a change in NMD, an increase or decrease in NMD activity indicating a modulating substance. The complex optionally comprises Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, Rrp41 or a combination thereof. Also disclosed are methods of the invention, wherein the complex comprises one or more of the group consisting of Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41. More specifically, the complex can comprise at least, two, three, four, five, six, seven, eight, nine, ten, eleven, twelve, thirteen, or fourteen members of the group consisting of Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41.

59. Disclosed are methods of screening for a substance that modulates nonsense-mediated mRNA decay (NMD) (optionally, during the pioneer round) comprising incubating the substance with a stably transfected cell comprising a reporter gene with a nonsense-mutation, and assaying the amount of NMD in the cell, wherein an increase or decrease in the amount of mRNA relative to the amount of mRNA in the absence of the substance indicates a substance that modulates NMD activity.

60. Disclosed are methods of screening for a substance that inhibits nonsense-mediated mRNA decay (NMD) comprising incubating the substance with Upf2 and Upf3 forming a substance-Upf2-Upf3 mixture, and assaying the amount of Upf2-Upf3 complex present in the mixture, wherein a decrease in the amount of Upf2-Upf3 complex relative to the amount of Upf2-Upf3 complex in the absence of the substance indicates that the substance inhibits NMD. 61. Also disclosed are methods of determining

if a substance promotes nonsense-mediated mRNA decay (NMD) (e.g., during the pioneer round) comprising incubating the substance with Upf2 and Upf3 forming a substance-Upf2-Upf3 mixture, and assaying the amount of Upf2-Upf3 complex present in the mixture, wherein an increase in the amount of Upf2-Upf3 complex relative to the amount of Upf2-Upf3 complex in the absence of the substance indicates that the substance promotes NMD.

62. As used throughout, when reference is made to a particular protein or nucleic acid, some variation in amino acid or nucleotide sequence is expected without a substantial decline in function. Thus, Upf2 can include proteins or nucleic acid sequences having at least 80%, 85%, 90%, or 95% identity to the sequence set forth in ATCC No. AF318574, or fragment thereof Also disclosed are methods of the invention, wherein the Upf3 has at least 80%, 85%, 90%, or 95% identity to the sequence set forth in ATCC No. AF318575, or fragment thereof.

63. Disclosed are methods of determining if a substance inhibits nonsense-mediated mRNA decay (NMD) comprising incubating the substance with Upf2 and Upf3X forming a substance-Upf2-Upf3X mixture, and assaying the amount of Upf2-Upf3X complex present in the mixture, wherein a decrease in the amount of Upf2-Upf3X complex relative to the amount of Upf2-Upf3X complex in the absence of the substance indicates that the substance inhibits NMD (optionally, during the pioneer round).

64. Also disclosed are methods of determining if a substance promotes nonsense-mediated mRNA decay (NMD) (e.g. during the pioneer round) comprising incubating the substance with Upf2 and Upf3X forming a substance-Upf2-Upf3X mixture, and assaying the amount of Upf2-Upf3X complex present in the mixture, whereinan increase in the amount of Upf2-Upf3X complex relative to the amount of Upf2-Upf3X complex in the absence of the substance indicates that the substance promotes NMD.

65. Disclosed are methods of determining if a substance that modulates nonsense-mediated mRNA decay (NMD) (e.g., the pioneer round of NMD) comprising incubating the substance with Upf2, Upf3, or Upf3X; and mRNA, and assaying the amount of Upf2, Upf3, or Upf3X that binds mRNA, wherein a increase or decrease in the amount of bound mRNA relative to the amount of bound mRNA in the absence of the substance indicates a substance that modulates NMD activity.

66. Disclosed are methods of screening for a substance that modulates nonsense-mediated mRNA decay (NMD) comprising, a) administering a substance to a system, wherein the system comprises at least the minimum components essential for NMD activity, b) assaying the effect of the substance on the amount of NMD activity in the system, and wherein a substance which causes a change in the amount of NMD activity present in the system compared to the amount of NMD activity in the system in the absence of composition is a modulator. In a preferred embodiment the method screens for a substance that modulates the pioneer round of NMD.

67. Disclosed are methods of modulating nonsense-mediated mRNA decay (NMD) activity (optionally, the pioneer round) comprising administering a substance, wherein the substance is identified by the methods of the invention.

68. The invention further provides methods of making a substance capable of modulating nonsense-mediated mRNA decay (NMD) activity comprising admixing a substance identified by the methods of the invention with a pharmaceutically acceptable carrier. In a preferred embodiment the substance is capable of modulating the pioneer round of NMD.

69. Also disclosed are substances made by methods of the invention.

70. Disclosed are methods of making a modulator of nonsense-mediated mRNA decay (NMD) (e.g. a modulation of the pioneer round) comprising a) administering a substance to a system, wherein the system comprises NMD activity, b) assaying the effect of the substance on the amount of NMD activity in the system, c) selecting a substance which causes a change in the amount of NMD activity present in the system compared to the amount of NMD activity in the system in the absence of substance, and d) synthesizing the substance.

71. More specifically, the invention discloses substances that modulate NMD in the pioneer round of translation. In one embodiment, the substance is an antibody that modulates NMD, optionally in the pioneer round. More specifically, the antibody can bind Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41. In another embodiment the substance is a siRNA that modulates NMD, preferably in the pioneer round. More specifically, the si RNA can bind Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41.

72. Also disclosed are substances of the invention, wherein the substance is a vector comprising a nucleic acid that encodes a-protein that modulates NMD, optionally in the pioneer round. Also disclosed is a cell comprising the vector of the invention.

73. Also disclosed are substances of the invention, wherein the protein is an antibody. The protein can optionally be an antibody, including e.g., an antibody that binds Upf2, Upf3, Upf3X, or PABP2.

74. Also disclosed are substances of the invention, wherein the substance is a chemical that modulates NMD, optionally, for example NMD in the pioneer round.

### C. COMPOSITIONS

75. Disclosed are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41 is disclosed and discussed and a number of modifications that can be made to a number of molecules including the Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41 are discussed, specifically contemplated is each and every combination and permutation of Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

76. Disclosed are substances that modulate NMD, optionally in the pioneer round of translation, including for example antibodies that modulate NMD, as described elsewhere in the application.

77. Also disclosed are substances of the invention, wherein the substance is a vector comprising a nucleic acid that encodes a protein (including, e.g., an antibody) that modulates NMD, optionally during the pioneer round.

78. Also disclosed are substances of the invention, wherein the substance is a chemical that modulates NMD, preferably the chemical modulates NMD during the pioneer round.

79. Also disclosed is a cell comprising the vector of the invention.

1. Sequence Similarities

80. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

81. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

82. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

83. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

84. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Nucleic Acids

85. There are a variety of molecules disclosed herein that are nucleic acid based, including for example the nucleic acids that encode, for example, Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41, as well as various functional nucleic acids. The disclosed nucleic acids are made up of for example, nucleotides, nucleotide analogs, or nucleotide substitutes. Non-limiting examples of these and other molecules are discussed herein. It is understood that, for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

86. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

87. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modifcation, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference for methods of modifying bases selectively.

88. Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modiifcations also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

89. Other modifications at the 2′ position include but are not limted to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO2 CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacolinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

90. Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

91. It is understood that nucleotide analogs need only contain a single modification, but may also contain multiple modifications within one of the moieties or between different moieties.

92. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

93. Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

94. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331;and 5,719,262 teach how to make and use PNA molecules, each of which is herein incorporated by reference. (See also Nielsen et al., Science, 1991, 254, 1497-1500).

95. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (anoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

96. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

97. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Functional Nucleic Acids

98. Functional nucleic acids are nucleic acid molecules that have a specific function, such as binding a target molecule or catalyzing a specific reaction (e.g., modulating NMD in the pioneer round). Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

99. Functional nucleic acid molecules can interact with any macromolecule, such as DNA, RNA, polypeptides, or carbohydrate chains. Thus, functional nucleic acids can interact with the mRNA of Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41 or the genomic DNA of Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41 or they can interact with the polypeptide Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp41. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

100. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant k_{d}) less than 10⁻⁶. It is more preferred that antisense molecules bind with a k_{d} less than 10⁻⁸. It is also more preferred that the antisense molecules bind the target moelcule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

101. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

3. Delivery of the Compositions to Cells

102. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosnids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

a) Nucleic Acid Based Delivery Systems

103. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

104. As used herein, plasmid or viral vectors are agents that transport the disclosed nucleic acids, such as Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp⁴¹ into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

105. Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

106. A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein.′ Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

107. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules that are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

108. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

109. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993);. Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

110. A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Asscociated Viral Vectors

111. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

112. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

113. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

114. The vectors of the present invention thus provide DNA molecules which are capable of integration into a mammalian chromosome without substantial toxicity.

115. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

116. Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

117. Other useful systems include, for example, replicating and host-restricted non-replicating vaccinia virus vectors.

118. Also disclosed are substances of the invention, wherein the substance is a vector comprising a nucleic acid that encodes a protein (e.g., an antibody which binds Upf2, Upf3, Upf3X, PAB2, CBP20, CBP80, hSmg5/7a, hSmg5/7b, hSmg5/7c, poly (A) ribonuclease (PARN), Dcp2, PM/Scl100, Rat1, Xrn1, or Rrp4l) that modulates NMD, preferably the pioneer round of NMD.

b) Non-Nucleic Acid Based Systems

119. The disclosed compositions can be delivered to the target cells in a variety of ways. For example, the compositions can be delivered through electroporation, through lipofection, or through calcium phosphate precipitation. The delivery mechanism chosen will depend in part on the type of cell targeted and whether the delivery is occurring for example in vivo or in vitro.

120. Thus, the compositions can comprise, in addition to the disclosed Upf2, Upf3, Upf3X, PABP2, CBP20, CBP80, CBP80, hSmg5/7a, hSmg5/7b, hSmg5/7c, poly (A) ribonuclease (PARN), Dcp2, PM/Scl100, Rat1, Xrn1, Rrp41 or vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No.4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

121. In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

122. The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as stealth and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

123. Nucleic acids that are delivered to cells that are to be integrated into the host cell genome typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

124. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In vivo/ex vivo

125. As described above, the compositions can be administered in a pharmaceutically acceptable carrier and can be delivered to the subjects cells in vivo and/or ex vivo by a variety of mechanisms well known in the art (e.g., uptake of naked DNA, liposome fusion, intramuscular injection of DNA via a gene gun, endocytosis and the like).

126. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

127. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

128. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIlI E restriction fragment (Greenway, P. J. et al., Gene 18: 355-360 (1982)). Of course, promoters from the host cell or related species also are useful herein.

129. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banedji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

130. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

131. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

132. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

133. Expression vectors used in eukaryotic host cells (yeast, fungi, insect, plant, animal, human or nucleated cells) may also contain sequences necessary for the termination of transcription that may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

134. The viral vectors can include a nucleic acid sequence encoding a marker product. This marker product is used to determine if the gene has been delivered to the cell and once delivered is being expressed. Preferred marker genes are the E. Coli lacZ gene, which encodes B-galactosidase, and green fluorescent protein.

135. In some embodiments the marker maybe a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DBFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DBFR or TK gene into cells lacking the respective genes, thus altering their growth requirements. Individual cells that were not transformed with the DHF or TK gene will not be capable of survival in non-supplemented media.

136. The second category is dominant selection which refers to a selection scheme used in any cell type and does not require the use of a mutant cell line. These schemes typically use a drug to arrest growth of a host cell. Those cells that have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Peptides

a) Protein Variants

137. As discussed herein there are numerous variants of the Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp41 proteins that are known and herein contemplated. In addition to the known functional Upf2, Upf3;, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp41 strain variants, there are derivatives of the Upf2, Upf3, Upf3X, PABP2, CBP20, CBP80, hSmg5/7a, hSmg5/7b, hSmg5/7c, poly (A).ribonuclease (PARN), Dcp2, PM/Scl100, Rat1, Xrn1, and Rrp41 proteins which also function in the disclosed methods and compositions. Protein variants and derivatives can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example, M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic | Glu |
| acidp | |
| serine | SerS |
| threonine | ThrT |
| tyrosine | TyrY |
| tryptophan | TrpW |
| valine | ValV |

TABLE 2
Conservative Amino Acid Substitutions

Ala; Ser
Arg; Lys; Gln
Asn; Gln; His
Asp; Glu
Cys; Ser
Gln; Asn; Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

138. Examples of conservative amino acid substitutions are shown in Table 2. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

139. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

140. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

141. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

142. It is understood that one way to define the variants and derivatives of the disclosed proteins herein is through defining the variants and derivatives in terms of homology/identity to specific known sequences. For example, ATCC ACCESSION NO.:AF318574 sets forth a particular sequence of Upf2 and ATCC ACCESSION NO.:AF318575 sets forth a particular sequence of a Upf3 protein. Specifically disclosed are variants of these and other proteins herein disclosed which have at least, 70% or 75% or 80% or 85% or 90% or 95% homology to the stated sequence. Methods of determining homology are described above.

143. It is understood that the description of conservative mutations and homology can be combined together in any combination, such as embodiments that have at least 70% homology to a particular sequence wherein the variants are conservative mutations.

144. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and, every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rip4l from which that protein arises is also known and herein disclosed and described.

6. Antibodies

a) Antibodies Generally

145. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies are tested for their desired activity using the in vitro assays described herein, or by analogous methods, after which their in vivo therapeutic and/or prophylactic activities are tested according to known clinical testing methods.

146. As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

147. The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRS) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CD Rs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

148. As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies that maintain CBP80, hSmg5/7a, hSmg5/7b, hSmg5/7c, PARN, Dcp2, PM/Scl100, Rat1, Xrn1, or Rrp41 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

149. Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

150. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

151. Monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

152. The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

153. In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

154. The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

155. As used herein, the term antibody or antibodies can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

156. The invention also provides fragments of antibodies which have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp41. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

157. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

158. A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

159. Also provided is an antibody reagent kit comprising containers of the monoclonal antibody or fragment thereof of the invention. The reagents can include, for example, fluorescent tags, enzymatic tags, or other tags. The reagents can also include secondary or tertiary antibodies or reagents for enzymatic reactions, wherein the enzymatic reactions produce a product that can be visualized.

b) Human Antibodies

160. The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boemer et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

161. The human antibodies of the invention can also be obtained from transgenic animals. For example, transgenic, mutant mice that are capable of producing a fall repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

c) Humanized Antibodies

162. Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

163. To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

164. Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are% also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

7. Administration of Substances

165. Substances of the invention including, e.g., antibodies, are preferably administered to a subject in a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

166. The antibodies or other substances can be administered to the subject, patient, or cell by injection (e.g., intravenous, intraperitoneal, subcutaneous, intramuscular), or by other methods such as infusion that ensure its delivery to the bloodstream in an effective form. Local or intravenous injection is preferred.

167. Effective dosages and schedules for administering the antibodies or other substances may be determined empirically, and making such determinations is within the skill in the art. Those skilled in the art will understand that the dosage of substances or antibodies that must be administered will vary depending on, for example, the subject that will receive the substance or antibody, the route of administration, the particular type of substance or antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above. Doses for other substances can be determined by one of skill in the art (see, e.g., Remington: The Science and Practice of Pharmacy (19th ed.) ed. A. R. Gennaro, Mack Publishing Company, Easton, Pa. 1995).

168. Following administration of an antibody or substance for treating, inhibiting, or preventing NMD, the efficacy of the therapeutic antibody can be assessed in various ways well known to the skilled practitioner. For instance, one of ordinary skill in the art will understand that an antibody of the invention that inhibits NMD is efficacious in treating or inhibiting an NMD in a subject by observing that the antibody increases mRNA levels or prevents further nonsense-mediated mRNA decay.

a) Nucleic Acid Approaches for Delivery

169. The proteins, polypeptides, or fragments thereof of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment.

b) Nucleic Acid Delivery

170. In the methods described above which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

171. As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods.

172. As one example, if the nucleic acid of the invention is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

173. Parenteral administration of the nucleic acid or vector of the present invention, if used, is generally characterized by injection. Injectables of any of the substances of the invention can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

8. Pharmaceutical Carriers/Delivery of Pharamceutical Products

174. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

175. The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

176. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach forparenteral administration involves use of a slow release or sustained release system such that a constant dosage is maintained. See, e.g., U.S. Pat. No. 3,610,795, which is incorporated by reference herein.

177. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Inmunother., 35:421-425, (1992); Pietersz and McKenzie, Immunolog. Reviews, 129:571-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

178. The compositions, including antibodies, can be used therapeutically in combination with a pharmaceutically acceptable carrier.

179. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

180. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

181. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

182. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

183. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

184. Compositions for oral administration include powders or granules, suspensions or solutions in water or non-aqueous media, capsules, sachets, or tablets. Thickeners, flavorings, diluents, emulsifiers, dispersing aids or binders may be desirable.

185. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

186. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

9. Chips and Micro Arrays

187. Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

188. Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

10. Compositions Identified by Screening with Disclosed Compositions/Combinatorial Chemistry

a) Combinatorial Chemistry

189. The disclosed compositions can be used as targets for any combinatorial technique to identify molecules or macromolecular molecules that interact with the disclosed compositions in a desired way. The nucleic acids, peptides, proteins and related molecules disclosed herein can be used as targets for the combinatorial approaches. Also disclosed are the compositions that are identified through combinatorial techniques or screening techniques in which the compositions disclosed in ATCC ACCESSION NOS. AF318576, AF318575, AFAF318574, U12137, AF116341, AF026029, X80030, X84157, U39665, AJ238969 or portions thereof, are used as the target in a combinatorial or screening protocol.

190. It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp4l, are also disclosed. Thus, the products produced using the combinatorial or screening approaches that involve the disclosed compositions, such as, Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp41, are also considered herein disclosed.

191. Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

192. There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

193. A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached-to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

194. Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice.

195. Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

196. Techniques for making combinatorial libraries and screening combinatorial libraries to isolate molecules that bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

197. Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits U.S. Pat. Nos. 5,698,685 and 5,506,337), sulfamides (U.S. Pat. No. 5,618,825), and benzodiazepines (U.S. Pat. No. 5,288,514).

198. Screening molecules for inhibition of Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, or Rrp⁴l is a method of isolating desired compounds.

199. In another embodiment the inhibitors are non-competitive inhibitors of Upf2, Upf3, Upf3X, PABP2, CBP20, CBP80, hSmg5/8a, hSmg5/7b, hSmg5/7c, poly (A) ribonuclease (PARN), Dcp2, PM/Scl100, Rat1, Xrn1, or Rrp41. One type of non-competitive inhibitor will cause allosteric rearrangements which prevent binding to target mRNA.

200. As used herein combinatorial methods and libraries included traditional screening methods and libraries as well as methods and libraries used in interative processes.

b) Computer Assisted Drug Design

201. The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

202. It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp41, are also disclosed. Thus, the products produced using the molecular modeling approaches that involve the disclosed compositions, such as, Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp41, are also considered herein disclosed.

203. Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

204. Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

205. A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

206. Although described above with reference to design and generation of compounds which could alter binding, one could also screen libraries of known compounds, including natural products or synthetic chemicals, and biologically active materials, including proteins, for compounds which alter substrate binding or enzymatic activity.

11. Kits

207. Disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits could include primers to perform the amplification reactions discussed in certain embodiments of the methods, as well as the buffers and enzymes required to use the primers as intended. The kits could include systems comprising the essential elements of NMD activity.

12. Compositions with Similar Funtions

208. It is understood that the compositions disclosed herein have certain functions, such as modulating NMD (e.g., the pioneering round of translation) or binding one or more components involved in NMD. Disclosed herein are certain structural requirements for performing the disclosed functions, and it is understood that there are a variety of structures which can perform the same function which are related to the disclosed structures, and that these structures will ultimately achieve the same result, for example stimulation or inhibition of No.

### D. METHODS OF MAKING THE COMPOSITIONS

209. The compositions disclosed herein and the compositions necessary to perform the disclosed methods can be made using any method known to those of skill in the art for that particular reagent or compound unless otherwise specifically noted.

1. Nucleic Acid Synthesis

210. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

211. One method of producing the disclosed proteins, such as ATCC ACCESSION NO.:AF318575 or a variant fragment thereof, is to link two or more peptides or polypeptides together by protein chemistry techniques. For example; peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

212. For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

213. Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Processes for Making the Compositions

214. Disclosed are methods of making a substance capable of modulating nonsense-mediated mRNA decay (NID) activity, preferably during the pioneer round, comprising admixing a substance identified by the methods of the invention with a pharmaceutically acceptable carrier. The invention also provides substances made by methods of the invention.

215. Disclosed are methods of making a modulator of nonsense-mediated mRNA decay (NMD) (e.g., in the pioneer round) comprising, a) administering a substance to a system, wherein the system comprises NMD activity, b) assaying the effect of the substance on the amount of NMD activity in the system, c) selecting a substance which causes a change in the amount of NMD activity present in the system compared to the amount of NMD activity in the system in the absence of substance, and d)synthesizing the substance.

216. Disclosed are cells produced by the process of transforming the cell with any of the disclosed nucleic acids. Disclosed are cells produced by the process of transforming the cell with any of the non-naturally occurring disclosed nucleic acids.

217. Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

218. Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

219. Also disclosed are animals produced by the process of adding to the animal any of the cells disclosed herein.

### E. METHODS OF USING THE COMPOSITIONS

1. Methods of Treatment

220. Disclosed are methods of treating a disorder in a subject comprising administering to the subject a substance, wherein the substance modulates nonsense-mediated mRNA decay, preferably in the pioneer round of translation. Modulation of nonsense-mediated mRNA decay in the pioneer round of translation that results in a descrease in NMD would increase the abundance of the nRNA containing a premature termination codon. Herein, modulation can be measured by, e.g., comparing the level of the mRNA harboring a premature termination codon relative to an unaffected cellular RNA to the level of that mRNA lacking a premature termination codon to the same unaffected cellular RNA.

221. Also disclosed are methods of the invention, wherein the disorder is an inherited genetic disorder. The genetic disorder can be selected from the group consisting of cystic fibrosis, hemophilia, Tay-Sachs disease, other mucopolysaccharidoses, Duchenne muscular dystrophy, other dystrophies, β°-thalassemia, other anemias, triose phosphate isomerase deficiency, other glycolytic enzyme deficiencies, Marfan Syndrome, other connective tissue disorders, ataxia telangiectasia, other DNA repair disorders, Alzheimer's disease, other dementias, Sandhoff disease, epidermolysis bullosa simplex, insulin resistance, maple syrup urine disease, hereditary fructose intolerance, X-linked severe combined immunodeficiency, other immunodeficiencies, inherited cancers such as those due to BRCA-1 nonsense mutations, carbohydrate metabolism disorders, amino acid metabolism disorders, lipoprotein metabolism disorders, lipid metabolism disorders, lysomal enzymes metabolism disorders, steroid metabolism disorders, purine metabolism disorders, pyrimidine metabolism disorders, metal metabolism disorders, porphyrin metabolism disorders, or heme metabolism disorders.

222. Also disclosed are methods of the invention, wherein the disorder is an acquired disorder, including, e.g., disorders from acquired mutations (such as a mutation in p53 or BRCA-1).

223. Also disclosed are methods of the invention, wherein the acquired disorder is a cancer. The cancer can be selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumours, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

224. The disclosed compositions can be used to treat any disease where uncontrolled cellular proliferation occurs, such as cancers. A non-limiting list of different types of cancers is provided above.

2. Methods of Using the Compositions as Research Tools

225. The compositions can be used for example as targets in combinatorial chemistry protocols or other screening protocols to isolate molecules that possess desired functional properties related to modulation of NMD (e.g., NMD in the pioneer round).

226. The disclosed compositions can also be used as diagnostic tools related to diseases listed above that result from nonsense-mutations affected by NMD including NMD in the pioneer round.

b 227. The disclosed compositions can be used as discussed herein as either reagents in microarrays or as reagents to probe or analyze existing microarrays. The disclosed compositions can be used in any known method for isolating or identifying single nucleotide polymorphisms. The compositions can also be used in any known method of screening assays, related to chip/microarrays. The compositions can also be used in any known way of using the computer readable embodiments of the disclosed compositions, for example, to study relatedness or to perform molecular modeling analysis related to the disclosed compositions.

228. Disclosed are methods of screening for a substance that modulates a nonsense-mediated mRNA decay (NMD) complex comprising incubating the substance with the complex, and assaying for a change in NMD, an increase or decrease in NMD activity indicating a modulating substance. Such a modulator preferably modulates NMD during the pioneer round.

229. Also disclosed are methods of the invention, wherein the complex comprises Upf2, Upf3, Upf3X, PABP2, CBP20, CBP80, hSmg5/7a, hSmg5/7b, hSmg5/7c, poly (A) ribonuclease (PARN), Dcp2, PM/Scl100, Rat1, Xrn1, and Rrp⁴¹ or any combination thereof.

230. Disclosed are methods of screening for a substance that modulates nonsense-mediated mRNA decay (NMD) preferably during the pioneer round, comprising incubating the substance with a stably transfected cell comprising a reporter gene with a nonsense-mutation, and assaying the amount of NMD in the cell, wherein a increase or decrease in the amount of mRNA relative to the amount of mRNA in the absence of the substance indicates a substance that modulates NMD activity.

231. Disclosed are methods of determining if a substance inhibits nonsense-mediated mRNA decay (NMD) comprising incubating the substance with Upf2 and Upf3X forming a substance-Upf2-Upf3X mixture, and assaying the amount of Upf2-Upf3X complex present in the mixture, wherein a decrease in the amount of Upf2-Upf3X complex relative to the amount of Upf2-Upf3X complex in the absence of the substance indicates that the substance inhibits NMD. Preferably the inhibition of NMD occurs in the pioneer round.

232. Disclosed are methods of screening for a substance that modulates nonsense-mediated mRNA decay (NMD) (preferably during the pioneer round) comprising, a) administering a substance to a system, wherein the system comprises the components essential for NMD activity, b) assaying the effect of the substance on the amount of NMD activity in the system, and wherein a substance which causes a change in the amount of NMD activity present in the system compared to the amount of NMD activity in the system in the absence of composition is a modulator.

3. Methods of Evaluating Expression of Genes Using Microarrays

233. The disclosed compositions and methods can be used to evaluate the expression of genes involved in NMD in the pioneer round of translation. Specifically contemplated are methods wherein mRNA from a system comprising a nonsense-mutation is assayed using a micro array. Genes identified as having significantly (as determined by the manufacturers specifications of the array) increased or decreased expression are comodulators of NMD.

4. Methods of Gene Modification and Gene Disruption

234. The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

235. One of the preferred characteristics of performing homologous recombination in mammalian cells is that the cells should be able to be cultured, because the desired recombination event occur at a low frequency.

236. Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal. Thus, animals can be produced that have target genes involved in NMD, preferably in the pioneer round.

5. Methods of Treating Cancer

237. As refered to throughout, when the treatment of cancer is mentioned, treatment of precancer conditions such as cervical dysplasia, anal dysplasias, other dysplasias, hyperplasias, atypical hyperplasias, and neoplasias is meant to be included.

### F. EXAMPLES

238. The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Nucleus-Associated NMD of β-globin mRNA Takes Place in Association with CBP80, CBP20, eIF4G, PABP2, and Upf2 and Upf3 Proteins

239. Considering that β-globin mRNA is subject to nucleus-associated NMD in non-erythroid cells such as HeLa and Cos cells (Kugler et al., 1995; Zhang et al., 1998a), β-globin mRNP was immunopurified to determine if NMD is evident using anti-CPB80 antibody. Endogenous CPB80 was assayed in order to avoid artifactual interactions that could be due to abnormally high levels of CPB80 produced from transiently introduced expression vectors. Therefore, only RNA substrates—either nonsense-free or nonsense-containing—derived from transiently introduced vectors. Notably, the detection of mRNP proteins in immunopurified complexes does not depend on the presence of these RNAs since they represent only a small fraction of cellular RNA. However, quantitative analysis of these RNAs in the complexes allows for identification of those proteins associated with NMD.

240. Cos cells were transiently transfected with two plasmids: a test pmCMV-Gl plasmid, which expressed β-globin (Gl) mRNA that was either nonsense-free (Norm) or harbored a premature termination codon at position 39 (Ter) within exon 2 (Zhang et. al., 1998a; FIG. 1A), and the reference phCMV-MUP plasmid (Belgrader and Maquat, 1994), which expressed mRNA for the mouse major urinary protein (MUP) and served to control for variations in the-efficiencies of cell transfection and RNA recovery. After 40 hr, cells were lysed. Lysates were first cleared by incubation with protein A-agarose beads and subsequently incubated with either anti-CBP80 antibody or, as a control, normal rabbit serum (NRS). After 90 min at 4EC, tRNA, RNase inhibitor and protein A-agarose beads were added, and the incubation was continued for another 60 min. The beads were washed extensively, bound material was eluted with SDS-containing buffer, and protein and RNA were purified for analysis by western blotting and RT-PCR, respectively.

241. The results of western blotting revealed that CBP80 was immunopurified using anti-CBP80 antibody but not NRS (FIG. 1B, upper), indicating that immunopurification was specific for CBP80-containing complexes. The results of RT-PCR revealed that Gl and MUP mRNAs were also immunopurified using anti-CBP80 antibody but not NRS (FIG. 1B, lower), offering additional evidence for the specificity of immunopurification. Quantitation of each CBP80-associated mRNA demonstrated that the level of nonsense-containing (Ter) Gl mRNA was reduced to 19±4% the level of nonsense-free (Norm) Gl rnRNA (FIG. 1B, lower). This level of reduction represented the full extent of NMD since the level of nonsense-containing Gl mRNA using total-cell mRNA (i.e., mRNA that had not been immunopurified) was reduced to essentially the same extent (i.e., 23±4% of nonsense-free Gl mRNA) (FIG. 1B, lower). These data indicate that the NMD of Gl mRNA takes place in association with CBP80.

242. The experiment was scaled up and repeated using anti-CBP80 antibody and, in parallel, anti-eIF4E antibody. Results of western blotting confirmed the specificity of each immunopurification by demonstrating that CBP80 but not eIF4E was immunopurified using anti-CBP80 antibody, eIF4E but not CBP80 was immunopurified using anti-eIF4E antibody, and neither protein was immunopurified using NRS (FIG. 1C, upper). Calculations indicated that 34±2% of cellular CBP80 was immunopurified using anti-CBP80 antibody, and 15±1% of cellular eIF4E was immunopurified using anti-eIF4E antibody. The finding that the NMD of Gl mRNA takes place in association with CBP80 was corroborated using RT-PCR: the level of nonsense-containing Gl mRNA was 17±4% and 20±4% of normal in complexes immunopurified using anti-CBP80 antibody and in total-cell RNA, respectively (FIG. 1C, lower). The level of nonsense-containing Gl mRNA was comparably reduced to 12±3% of normal in complexes immunopurified using anti-eIF4E antibody (FIG. 1C, lower). These results demonstrate that more than 80% of CBP80-bound Gl mRNA is degraded when the mRNA harbors the nonsense codon. Since CBP80 is thought to be replaced by eIF4E in the cytoplasm (Visa et al., 1996; Shen et al., 2000), and the Gl mRNA that escapes NMD can be available for binding to eIF4E.

243. Additional evidence for specificity of the immunopurifications and the idea that CBP80-bound mRNA is susceptible to NMD was provided by the findings that complexes immunopurified using anti-CBP80 antibody but not anti-eIF4E antibody contained four other proteins detectable by western blotting: CBP20, which together with CBP80 forms the heterodimeric nuclear cap binding complex, poly(A) binding protein II (PABP2), which binds newly synthesized poly(A) tails of pre-mRNA and mRNA, and Upf2 and Upf3 proteins (FIG. 1C, upper). Since Upf2 and Upf3 proteins are known to be involved in NMD, their presence in CBP80-containing but not eIF4E-containing complexes offers strong evidence that Gl mRNA is a substrate for NMD when bound by CPB80 and is not detectably a substrate when bound by eIF4E. The presence of Upf2 and Upf3 proteins as well as PABP2 in CBP80-containing complexes was dependent on RNA as evidenced by their absence in immunopurified complexes that were treated with RNase A prior to dissociation from anti-CBP80 antibody. In contrast, the presence of CPB20 in CBP80-containing complexes was not dependent on RNA, as would be expected considering its direct interaction with CPB80. Interestingly, CBP20, PABP2 and Upf3 protein are mostly nuclear, consistent with the newly synthesized nature of both CBP80-containing mRNP and nucleus-associated NMD. However, PABP2 and Upf3 proteins shuttle, opening up the possibility that nucleus-associated NMD could take place on the cytoplasmic side of the nuclear envelope. In support of this possibility, Upf2p is not detected in nuclei (Lykke-Andersen et al., 2000). Upf1 protein, which is known to interact with Upf2 and Upf3 proteins in HeLa cells, was not detected in either CBP80-bound or eIF4E-bound Gl mRNA (FIG. 1C, upper). Its absence from CPB80-bound mRNA is consistent with its putative role in NMD only after translation termination. Both CBP80 and eIF4E were associated with eIF4G and ribosomal protein L10 (FIG. 1C, upper), consistent with the translational dependence of NMD (reviewed in Maquat, 2000). Notably, the amounts of eIF4G and L10 associated with CBP80 were small but reasonable considering the amounts associated with eIF4E, which supports the bulk of cellular translation.

244. As further indications that the CBP80-bound Gl mRNP complexes under evaluation realistically represent complexes formed in vivo, mRNP immunopurified using anti-CBP80 antibody was isolated from nuclear and cytoplasmic fractions of transfected cells. Nuclear fractions were not contaminated by cytoplasm as indicated by the absence of detectable eIF4A, which has been shown to be undetectable in nuclei (Lejbkowicz et al., 1992). CBP80-bound Gl mRNA was detected by RT-PCR in both nuclear and cytoplasmic fractions (FIG. 2), consistent with CBP80 being a shuttling protein. Furthermore, the extent of NMD for CBP80-bound Gl mRNA in both cellular fractions was comparable (FIG. 2). This result is expected given that Gl mRNA is subject to nucleus-associated NMD (Zhang et al., 1998; Thermann et al., 1998), and is consistent with the idea that NMD takes place in association with CBP80.

245. As another means of confirming that the CBP80-containing complex is a substrate for NMD, mRNP was immunopurified from untransfected cells using anti-Upf3/3X antibody. This antibody reacts with Upf3 and Upf3X proteins, which derive from non-X-linked and X-linked UPF3 genes, respectively (Lykke-Andersen et al., 2000; Serin et al., 2001). Notably, both Upf3 and Upf3X proteins are mostly nuclear and shuttle between nuclei and cytoplasm (Lykke-Andersen et al., 2000; Serin et al., 2001). Consistent with findings described above, CPB80, PABP2 and Upf2 protein but not eIF4E or Upf1 protein were detected in complexes immunopurified using anti-Upf3/3X antibody (FIG. 3). The presence of CPB80 and the absence of eIF4E in complexes immunopurified using anti-Upf3/3X antibody corroborate the idea that NMD is largely confined to CBP80-bound mRNA. The finding that there is more Upf2 protein in complexes immunopurified using anti-Upf3/3X antibody than in complexes immunopurified using anti-CBP80 antibody is consistent with data demonstrating that Upf2 and Upf3 proteins can interact independently of RNA (Lykke-Andersen et al, 2000) whereas the interaction between CBP80 and Upf3 is dependent on RNA.

b) Evidence that CPB80-Bound Gl mRNA is Translated

246. The finding that NMD takes place in association with CBP80 indicates that CBP80-bound mRNA can be translated, which is consistent with the presence of eIF4G and ribosomal protein in immunopurifications using anti-CBP80 antibody (FIG. 1C). To obtain more direct evidence for the translatability of CBP80-bound mRNA, the effect of a block in translation on the level of total-cell CBP80-bound mRNA was quantitated. Addition of 100:g/ml of cycloheximide for two hr prior to cell harvesting abrogated the NMD of CBP80-bound Gl mRNA so that the level of nonsense-containing mRNA was increased from 15% to 42% of normal (FIG. 4A). Therefore, CBP80-bound mRNA is a substrate for protein synthesis. This conclusion was corroborated using an amber suppressor tRNA (Belgrader et al., 1993) that directs the incorporation of serine at the nonsense codon of nonsense-containing Gl mRNA. Co-expression of a plasmid producing suppressor tRNA abrogated the NMD of CBP80-bound Gl mRNA: the level of nonsense-containing Gl mRNA was increased from 23% of normal to 37%, 46% or 62% of normal as the ratio of suppressor tRNA expression vector to Gl mRNA expression vector was increased (FIG. 4B). In contrast, co-expression of a plasmid harboring the suppressor tRNA gene in antisense orientation had no effect on the NMD of CBP80-bound Gl mRNA (FIG. 4B).

c) Cytoplasmic NMD of GPx1 mRNA Also Takes Place in Association with CBP80, CBP20, eIF4G, PABP2, and Upf2 and Upf3 Proteins

247. Unlike nucleus-associated NMD, for which data are lacking, cytoplasmic NMD has been demonstrated to take place on cytoplasmic ribosomes (Rajavel and Neufeld, 2001). In order to gain insight into the mechanistic relationship between NMD at the two cellular sites, the cap binding protein associated with mRNA that is a substrate for cytoplasmic NMD was determined. To this end, Cos cells were transiently transfected with two plasmids: a test pmCMV-GPx1 plasmid, which expressed glutathione peroxidasel (GPx1) mRNA that was either nonsense-free (Norm) or harbored a premature termination codon at position-46 (Ter) within exon 1 (Moriarty et al., 1998; FIG. 5A), and the reference phCMV-MUP plasmid. Immunopurifications were performed using either anti-CBP80 or anti-eIF4E antibody as described above for studies of Gl mRNP. 248. The results of western blotting confirmed the specificity of each immunopurification, i.e., the absence of detectable eIF4E in the immunopurification using anti-CBP80 antibody, the absence of detectable CBP80 in the immunopurification using anti-eIF4E antibody, and the absence of either protein in the immunopurification using NRS (FIG. 5B, upper). Furthermore, quantitation of GPx1 and MUP mRNAs by RT-PCR revealed the presence of each mRNA in immunopurifications using anti-CBP80 or anti-eIF4E antibody but not NRS (FIG. 5B, lower). Remarkably, despite its cytoplasmic location, NMD was evident for CBP80-bound GPx1 mRNA: the level of nonsense-containing mRNA was 25±5% of normal, which was comparable to the (29±4% of normal) level of nonsense-containing NIRNA that had not undergone immunopurification (FIG. 5B, lower). The level of nonsense-containing mRNA bound by eIF4E was 21±2% of normal (FIG. 5B, lower), indicating that GPx1 mRNA may no longer be appreciably susceptible to NMD once CBP80 is replaced by eIF4E.

249. Also consistent with findings using Gl mRNA, results of western blotting revealed that CBP80-bound GPx1 mRNA but not eIF4E-bound GPx1 mRNA was associated with CBP20, PABP2, Upf2 protein and Upf3 protein (FIG. 5B, upper). Furthermore, both CBP80-bound and eIF4E-bound GPx1 mRNAs were associated with e]F4G and ribosomal protein L10, and neither was detectably associated with Upf1 protein (FIG. 5B, upper). The analysis of cell fractions was used to corroborate the biological significance of the GPx1 mRNP under study. NMD was evident for CBP80-bound GPx1 mRNA in the cytoplasmic but not nuclear fraction (FIG. 6). This result is consistent with the fact that NMD of GPx1 mRNA takes place only after export to the cytoplasm and indicates that translation in the cytoplasm can take place in association with CBP80.

d) Discussion

250. It was determined that mRNA bound by the cap binding proteins CPB80 and CBP20 and the poly(A) binding protein PABP2 is translated and subject to NMD regardless of whether NMD takes place in association with nuclei or in the cytoplasm. Even though CBP80, CBP20 and PABP2 are primarily nuclear, their function in cytoplasmic NMD is attributable to their ability to shuttle between nuclei and cytoplasm. In the cytoplasm, the replacement of CPB80 and CPB20 by the primarily cytoplasmic cap binding protein eIF4E is generally concomitant with the loss of susceptibility to NMD for both nucleus-associated and cytoplasmic NMD. Furthermore, Upf2 protein, which is also bound to mRNA subject to NMD at either cellular site, is detected only in the cytoplasm albeit mostly at the nuclear periphery (Lykke-Andersen et al., 2000; Serin et al., 2001). Additionally, Upf1 protein, which is known to be required for NMD (Sun et al., 1998) and interacts with Upf2 protein (Serin et al., 2001), is also detected only in the cytoplasm (Lykke-Andersen et al., 2000; Serin et al., 2001).

251. These findings are important for a number of reasons. Most notably, CBP80, CBP20 and, presumably, PABP2 can now be defined as components of the translation initiation complex that typifies early rounds—possibly exclusively the first round—of translation in mammalian cells. The idea of a “pioneer” round that is distinct from subsequent rounds has recently been indicated for transcription by RNA polymerase II (reviewed in Orphanides and Reinberg, 2000). A pioneer round of translation during which NMD generally functions certainly makes sense considering that NMD surveys mRNAs to eliminate those that prematurely terminate translation as a form of quality control.

252. In the steady-state initiation complex, eIF4G is a multipurpose adaptor, interacting directly with eIF4E, eIF3, eIF4A and PABP1 (reviewed in Hentze et al., 1997; Gingras et al., 1999; Sachs and Varani, 2000; see FIG. 7). For a comparable complex to exist during the pioneer round of translation, CBP80-CBP20 would interact with eIF4G analogously to the way eIF4E interacts with eIF4G; similarly, PABP2 would functionally replace PABP1 (FIG. 7).

253. Consistent with recent studies of mammalian cells demonstrating that CBP80 and eIF4G co-immunoprecipitate (McKendrick et al., 2001), recent studies of S. cerevisiae demonstrated that eIF4Gl interacts directly with CBP80 in vitro in a way that is competed by eIF4E (Fortes et al., 2000). Additionally, TAP-tagged CBP20 co-purified with CBP80, eIF4Gl and a small amount of eIF4E, indicating an association in extracts.

e) Materials and Methods

(1) Cell Culture, Plasmids and Transfection

254. Monkey kidney Cos-7 and mouse NIH3T3 cells were cultured in MEM-α (Gibco BRL, Grand Island, N.Y.) supplemented with 10% FCS. Cells were transfected with test plasmids pmCMV-Gl (Norm or 39Ter; Sun et al. 1998) or pmCMV-GPx1 Norm or 46Ter, also called 46 TAA; Moriarty et al. 1998) and reference plasmid phCMV-MUP (Belgrader and Maquat, 1994) by using calcium phosphate (Wigler et al., 1979). Where indicated, cycloheximide (100 μg/ml; Sigma, St. Louis, Mo.) was added 2 hour before harvesting. Alternatively, either p53tsSu+, which expresses an amber suppressor tRNA (Belgrader et al., 1993), or a derivative of p53tsSu+ that harbored the amber suppressor tRNA in antisense orientation was included in the transfection.

(2) Antibodies to Human Upf2 and Upf3 Proteins

255. Rabbit polyclonal antisera were raised (Rockland, Inc.) against His6-tagged human Upf2 protein (Serin et al., 2001; amino acids 441-761) and His6-tagged human Upf3 protein (Serin et al., 2001; amino acids 1-177). Each antibody was purified using protein A agarose (Sigma, St. Louis, Mo.) following the protocol described in Harlowe and Lane (1999).

(3) Cell Lysis and Immunopurifications

256. Immunopurification and RT-PCR were according to Chu et al. (1999), with modification. Briefly, two days after transfection, 3.2×10⁷ Cos-7 cells or, where indicated, NIH3T3 cells, were washed three times with ice-cold phosphate-buffered saline (PBS). Cells were harvested and suspended in PBS. After centrifugation, cell pellets were resuspended in 0.5 ml of NET-2 buffer (50 mM Tris-HCl, pH 7.4, 300 mM NaCl, 0.05% NP-40) containing 100 U of RNase-inhibitor (Promega, Madison, Wis.). Cells were sonicated with three 10-sec bursts using a Branson Sonifier. After sonication, homogenates were centrifuged at 10,000×g for 10 min at 4° C. The supernatants were used as the source of antigen in the immunopurifications. They were first cleared by incubation with end-over-end rotation in the presence of 100 μl of protein A-agarose beads (Boehringer Mannheim, Indianapolis, Ind.) for 30 min at 4° C. followed by centrifugation at 10,000×g. Cleared supernatants were then rotated in the presence of a specific anti-serum for 90 min at 4° C., after which 0.5 mg of yeast tRNA (Sigma, St. Louis, Mo.), 100 U of RNase-inhibitor, and protein A-agarose beads was added. The incubation was continued for another 60 min at 4° C. The beads were washed six times with NET-2 buffer, suspended in 50 μl of SDS sample buffer (0.1 M Tris-HCl, pH 6.8, 4% SDS, 20% glycerol, 12% 2-mercaptoethanol), and split into two portions. In some experiments, RNase A (20 μg; Sigma, St. Louis, Mo.) or, as a control, BSA (20 μg; New England Biolabs) was added to the beads immediately prior to splitting. One portion (six-tenths) was used as a source of protein, which was analyzed by SDS-PAGE and western blotting. The other portion (four-tenths) was extracted with phenol, chloroform and isoamyl alcohol, and precipitated using ethanol. Precipitates were dissolved in 50 μl of NET-2 buffer, treated with 10 U of DNase I (Promega, Madison, Wis.) for 15 min at 37° C., extracted, precipitated, dissolved in 30 :l of water, and used as a source of RNA for RT-PCR (Sun et al. 1998). Notably, this protocol was scaled up to include as many as eight individual immunopurifications, which were pooled as a source of protein and RNA. In cases where nuclear and cytoplasmic fractions were analyzed, nuclei and cytoplasm were separated from 1-2×10⁸ transfected cells as previously described (Belgrader et al. 1994).

(4) Western Blot Analysis

257. Immunopurified protein or protein prior to immunopurification (5-10 :l) was electrophoresed in 8% (eIF4G, Upf1 protein or Upf2 protein), 10% (CBP80); 12% (eIF4E or CBP20),15% (rpL10), or a gradient of 4-15% polyacrylamide (Bio-Rad) either with (Upf3 protein) or without (PABP2)12% 2-mercaptoethanol. Protein was transferred to Hybond ECL nitrocellulose (Amersham), and probed with antibody against CBP80 (Izaurralde et al., 1994), CBP20 (Izaurralde et al., 1995), eIF4E (Morley and McKendrick, 1997), eIF4G (Morley and McKendrick, 1997), PABP2 (Krause et al., 1994), Upf1 protein (Pal et al., 2001), Upf2 protein (Serin et al., 2001), Upf3 protein (Serin et al., 2001) or ribosomal protein (rp) L10 (QM rabbit polyclonal IgG; Santa Cruz Biotechnology). In the case of cellular fractionations, nuclear fractions were deemed free of cytoplasm by that absence of detectable reactivity with antibody against eIF4A (Lejbkowicz et al., 1992). Reactivity to each primary antibody was detected using a 1:5000 dilution of horse radish peroxidase-conjugated donkey anti-rabbit antibody (Amersham). Reactivity of the secondary antibody was visualized by SuperSignal West Pico or West Femto Solution (Pierce, Rockford, Ill.).

(5) RT-PCR

258. Immunopurified Gl, GPx1 or MUP transcripts (10 :l) or transcripts prior to immunopurification were analyzed by RT-PCR as described (Sun et al., 1998). GPx1 RNA was amplified using the primers 5′ ACCACCGTAGAACGCAGATCG 3′ (sense) and 5′ CTTCTCACCATTCACCTCGCACTT 3′ (antisense). Gl RNA was amplified using the same sense primer that was used to amplify Gl RNA and 5′ GGGTTTAGTGGTACTTGTGAGC 3′ (antisense). MUP RNA was amplified using 5′ CTGATGGGGCTCTATG 3′ (sense) and 5′ TCCTGGTGAGAAGTCTCC 3′ (antisense). Half of each RT-PCR reaction was electrophoresed in 5% polyacrylamide. The simultaneous analysis of serial dilutions of RNA ensured that the RT-PCR was quantitative. RT-PCR products were quantitated by PhosphorImaging (Molecular Dynamics).

### 2. Example 2

259. The goal of this experiment was to gain a broader understanding of the structural rearrangements that typify CBP80- and eIF4E-bound transcripts as a consequence of pre-mRNA synthesis, pre-mRNA splicing, mRNA export and mRNA translation. The characterization of proteins bound to spliced mRNA immunopurified from nuclear and cytoplasmic fractions of Cos cells using anti-CBP80, anti-eIF4E or anti-Upf3/3X antibody under conditions that preserve RNP is reprted here. In nuclear fractions, anti-CBP80 antibody and anti-Upf3/3X antibody, but not anti-eIF4E antibody, immunopurified RNPS1, Y14, SRm160 and REF/Aly, all of which are components of the EJC. Also present in the anti-CBP80 and anti-Upf3/3X immunopurifications of nuclear fractions were the NMD factors Upf3X and Upf2 and the mRNA export factor TAP. RNase treatment demonstrated that immunopurification of RNPS1, Y14, SRm160, REF/Aly and Upf2 but not CBP80 using anti-Upf3/3X antibody was independent of RNA, consistent with these proteins constituting the EJC and associating with Upf3/3X. Furthermore, immunopurification of RNPS1, Y14, Upf3X, Upf2 and, to a lesser extent, SRm160 and REF/Aly using anti-CBP80 antibody was dependent on RNA, indicating that SRm160 and REF/Aly, in addition to being part of the EJC, can interact with CBP80. The immunopurification of TAP using anti-Upf3/3X antibody was also dependent on RNA, indicating that TAP has a relatively weak association with the EJC, is present in association with non-EJC mRNA binding proteins, or both. All EJC components were detected in cytoplasmic fractions immunopurified using anti-CBP80 antibody in a way that is RNase sensitive, indicating that they are exported with mRNA from the nucleus to the cytoplasm before completely dissociating. Data demonstrating that Upf2 and Upf3X are exported to the cytoplasm in association with CBP80 and the EJC are consistent with the understanding that some mRNAs are subject to cytoplasmic NMD. The finding that eIF4E-bound transcripts that co-purify with nuclei are not bound by the EJC, together with the detection of significant levels of CBP80-bound pre-mRNA but not eIF4E-bound pre-mRNA, indicate that eIF4E replaces CBP80/CBP20 concomitant with or after dissociation of the EJC. In support of the idea that CBP80/CBP20 rather than eIF4E binds to nascent transcripts, antibody against the C-terminal domain of RNA polymerase II immunopurified CBP80 but not eIF4E.

a) β-Globin mRNA that Co-Purifies with Nuclei is Bound by CBP80 or eIF4E

260. CBP80 together with CBP20 comprise the major nuclear cap binding complex that is added co-transcriptionally and functions in nuclear RNA processes such as pre-mRNA splicing and 3′ end formation (zaurralde et al., 1994; Lewis and Izaurralde, 1997). Like CBP80 in C. tentans (Visa et al., 1996) and S. cerevisiae (Shen et al., 2000), mammalian CBP80 is a nucleocytoplasmic shuttling protein (Ishigaki et al., 2001). Given that the bulk of cellular translation involves mRNA bound by the major cytoplasmic cap binding protein, eIF4E, it makes sense that eIF4E replaces CBP80 at some point after mRNA export to the cytoplasm (reviewed in Gingras et al., 1999). However, the finding that a fraction of cellular eIF4E localizes to the nucleus (Lejbkowitz et al., 1992; Dostie et al., 2000) indicates that eIF4E could also have a nuclear function.

261. To determine whether eIF4E that co-purifies with nuclei associates with mRNA, Cos cells were transiently transfected with two plasmids: a test pmCMV-Gl plasmid that produces β-globin (Gl) mRNA (Zhang et al., 1998; FIG. 8A), and the reference phCMV-MUP plasmid that produces mRNA for the mouse major urinary protein (MUP; Belgrader and Maquat, 1994) and served to control for variations in the efficiencies of cell transfection and RNA recovery. After 40 hr, cells were lysed, nuclear and cytoplasmic fractions were generated, and immunopurifications were performed under conditions that preserve RNP (Ishigaki et al., 2001). Briefly, lysates were first cleared by incubation with protein A-agarose beads and subsequently incubated with rabbit anti-CBP80 antibody, rabbit anti-eIF4E antibody or, as a control for non-specific immunopurification, normal rabbit serum (NRS). After 90 min at 4° C., yeast RNA and protein A-agarose beads were added, and the incubation was continued for another 60 min. The beads were washed extensively, bound material was eluted with SDS-containing buffer, and protein and RNA were purified for analysis by western blotting and RT-PCR, respectively.

262. Results obtained using western blotting were consistent with previous findings (Ishigaki et al., 2001): anti-CBP80 antibody immunopurified only CBP80 in both cellular fractions, anti-eIF4E antibody immunopurified only eIF4E in both cellular fractions, and the control NRS did not immunopurify either protein in either cell fraction (FIG. 8B). RT-PCR demonstrated that each antibody immunopurified Gl and MUP mRNAs and that, regardless of the cellular fraction or cap binding protein, the level of nonsense-containing (Ter) Gl mRNA was reduced to an average of 17±7% the level of nonsense-free (Norm) mRNA (FIG. 8C). Therefore, eIF4E is associated with mRNA that co-purifies with nuclei.

b) CBP80 but not eIF4E is Detectably Bound to Intron-Containing Gl and SV40 T Antigen pre-mRNAs.

263. Since CBP80-bound but not eIF4E-bound mRNA was found to be, associated with factors required for NMD, the reduction in the abundance of nonsense-containing eIF4E-bound mRNA was attributed to NMD that occurred prior to the exchange of CBP80 for eIF4E (Ishigali et al., 2001). Therefore, intron-containing pre-mRNA can be bound primarily by CBP80 rather than eIF4E. Alternatively, it can be bound by CBP80 or eIF4E but the latter is a dead-end product that fails to be spliced and, therefore, fails to recruit Upf3/3X and Upf2 (Ishigaki et al., 2001; see below). One way to determine whether eIF4E-bound mRNAs generally derive from CBP80-bound mRNAs or, alternatively, eIF4E binds to pre-mRNA is to identify the cap binding protein on intron-containing RNA: if it is only CBP80, then eIF4E-bound RNA must derive from CBP80-bound RNA. To this end, the experiment described above was repeated. However, only nuclear fractions were prepared, and intron-containing Gl pre-mRNA was analyzed instead of Gl mRNA. Furthermore, the anti-eIF4E antibody derived from either rabbit or mouse. When mouse anti-eF4E antibody was used, the level of non-specific immunopurification was controlled for using mouse IgG.

264. RT-PCR demonstrated that the levels of MUP mRNA immunopurified using either anti-CBP80 antibody or mouse anti-eIF4E antibody were higher than non-specific, i.e., higher than the levels immunopurified using NRS or mouse IgG, respectively (FIG. 9A), consistent with previous findings (Ishigaki et al., 2001; FIG. 8). In contrast, while the level Gl pre-mRNA immunopurified using anti-CBP80 antibody was higher than non-specific, the level of Gl pre-mRNA immunopurified using mouse anti-eIF4E antibody was barely above non-specific (FIG. 9A). The essential failure to detect eIF4E-bound Gl pre-mRNA was reproduced using rabbit anti-eIF4E antibody (FIG. 9B). Therefore, CBP80 is readily detected in association with Gl pre-mRNA whereas eIF4E is not.

265. To determine if this result can be extended to another pre-mRNA, the immunopurification was repeated using untransfected Cos cells. RT-PCR was then used to assay the relative levels of SV40 T antigen (ag) pre-mRNA and mRNA. The levels of SV40 T ag mRNA immunopurified using either anti-CBP80 or mouse anti-eIF4E antibody were higher than non-specific (FIG. 9C). However, as was the case for Gl pre-mRNA, the level of SV40 T ag pre-mRNA immunopurified using anti-CBP80 antibody was significantly higher than non-specific, while the level immunopurified using mouse anti-eIF4E antibody was only slightly higher than non-specific (FIG. 9C).

266. Therefore, the vast majority of Gl and SV40 T ag pre-mRNAs is bound by CBP80 rather than eIF4E, indicating that eIF4E-bound mRNA derives primarily from transcripts initially bound by CBP80.

c) CBP80 but not eIF4E Co-Immunopurifies with the Carboxyl-Terminal Domain of RNA Polymerase II

267. Given that 5′ capping enzymes bind the phosphorylated carboxyl-terminal domain (CTD) of transcribing RNA polymerase II molecules (McCracken et al., 1997; Schroeder et al., 2000; Pei et al., 2001), and since proteins known to function in pre-mRNA splicing and 3′-end formation also associate with the CTD (reviewed in Hirose and Manley, 2000; Proudfoot, 2000), an additional way to determine whether CBP80-bound mRNA is generally a precursor to eIF4E-bound mRNA might be to determine whether CBP80 or eIF4E also co-immunopurify with the CTD. To test this idea, the nuclear fraction of untransfected Cos cells was immunopurified using anti-CTD antibody, and the presence of CBP80 and eIF4E was tested.

268. The results of western blotting indicate that anti-CTD antibody immunopurified CTD, as expected and, remarkably, CBP80 but not eIF4E (FIG. 10). This finding is consistent with the finding that intron-containing pre-mRNA is bound by CBP80 rather than eIF4E (FIG. 9) and offers additional support for a precursor-product relationship between CBP80-bound mRNA and eIF4E-bound mRNA. Another protein that failed to immunopurify with the CTD was Upf2 (FIG. 10), which was chosen as a negative control based on its cytoplasmic localization (Lykke-Anderson et al., 2000; Serin et al., 2001).

d) Anti-CBP80 Antibody, Unlike Anti-eIF4E Antibody, Co-Immunopurifies with RNPS1, Y14, Srm160, REF/Aly, TAP, Upf2 and Upf3X in Nuclear and Cytoplasmic Fractions

269. To gain insight into the dynamics of mRNP remodeling before and after eIF4E replaces CBP80 at the mRNA cap, the immunopurifications were repeated using nuclear and cytoplasmic fractions from untransfected Cos cells and analyzed for a variety of proteins using western blotting. Initially, RNPS1, Y14, SRm160, REF/Aly and DEK, which are components of the mRNA EJC that is deposited as a consequence of pre-mRNA splicing, were analyzed. Remarkably, all components except DEK were detected in immunopurifications of both nuclear and cytoplasmic fractions using anti-CBP80 antibody (FIG. 11). The failure to detect DEK indicates that either it is not present in the complex in vivo or that conditions used in the immunopurification removed DEK. A similar conclusion was drawn from immunopurifications using unfractionated mammalian cells expressing FLAG-tagged proteins, where all tagged components of the EJC were detected except for DEK (Lykke-Andersen et al., 2001). Our results indicate that RNPS1, Y14, SRm160 and REF shuttle to the cytoplasm, presumably in association with spliced mRNA (see below). Considering that SRm160 does not shuttle between the two nuclei of mammalian-cell heterokaryons (Lykke-Andersen et al., 2001), detection of SRm160 in the cytoplasmic fraction by immunopurification using anti-CBP80 antibody indicates that SRm160 can have a limited trajectory in the cytoplasm. Consistent with our failure to detect eIF4E-bound pre-mRNA, no components of the EJC were detected in either cell fraction using anti-eIF4E antibody (FIG. 11).

270. The mRNA export factor TAP and the NMD factors Upf2 and Upf3X were also detected in anti-CBP80 antibody immunopurifications of nuclear and cytoplasmic fractions but not anti-eIF4E immunopurifications of either fraction (FIG. 11). The failure to detect either Upf protein using anti-eIf4E antibody is consistent with data demonstrating that eIF4E-bound mRNA is largely immune to NMD (Ishigaki et al., 2001). SMG1/ATX, the PIK-related protein kinase thought to be required for NMD because of its role in phosphorylating Upf1 (Denning et al., 2001; Yamashita et al., 2001), was not detected in either anti-CBP80 or anti-eIF4E immunopurification (FIG. 11). This indicates that, like Upf1, SMG1/ATX is not a stable component of mRNP, at least under the purification conditions used here.

e) Evidence that RNPS1, Y14, SRm160, REF/Aly, TAP, Upf2 and Upf3X are Components of or Associate with the EJC on CBP80-Bound mRNA in Mammalian Cells

271. In order to determine if RNPS1, Y14, SRm160, and REF/Aly immunopurify with anti-CBP80 antibody as components of the EJC of mRNP, the immunopurifications were repeated using anti-Upf3/3X antibody or anti-CBP80 antibody either with or without the addition of RNase prior to the immunopurification step. Data demonstrating that Upf3/3X is recruited to mRNP by an EJC formed in vitro in mammalian-cell extract (Kim et al., 2001) or in vivo in intact mammalian cells using FLAG-tagged Upf3 produced by transient transfect (Lykke-Andersen et al., 2001) indicate that the EJC and CBP80 bind to distinct regions of mRNA. Therefore, components of or proteins recruited by the EJC would be expected to immunopurify with anti-Upf3/3X antibody both before and after RNase treatment but with anti-CBP80 antibody only before and not after RNase treatment.

272. RNase treatment was shown to be effective by the disappearance of β-actin mRNA in immunopurified samples after exposure to RNase (FIG. 12A). As expected, anti-Upf3/3X antibody immunopurified Upf3X in both nuclear fractions with or without exposure to RNase (FIG. 12B) and anti-CBP80 antibody immunopurified CBP80 in total-cell extract with or without exposure to RNase (FIG. 12C). Furthermore, anti-Upf3/3X antibody immunopurified CBP80 in both fractions only in the absence of RNase but immunopurified Upf2, RNPS1 and Y14 in both fractions in the absence or presence of RNase (FIG. 12B). These results, together with the finding that anti-CBP80 antibody immunopurified Upf3X, Upf2, RNPS1 and Y14 in total-cell extract only in the absence of RNase (FIG. 12C), indicate that Upf3X and Upf2 form a complex with the EJC that is conserved after mRNA export to the cytoplasm. SRm160 and REF/Aly immunopurified with anti-Upf/3X antibody in the nuclear fraction with or without exposure to RNase (FIG. 12B), as would be expected of EJC components. However, they failed to immunopurify with anti-Upf3/3X antibody in the cytoplasmic fraction (FIG. 12B), and they immunopurified with anti-CBP80 antibody in a partially RNase-insensitive manner (FIG. 12C). Considering that both proteins immunopurify with anti-CBP80 antibody in the cytoplasmic fraction (FIG. 11), they undergo a remodeling in the cytoplasm that provides a more direct connection to CBP80. The finding that TAP immunopurifies with anti-Upf3/3X antibody and anti-CBP80 antibody in an RNase-sensitive manner in nuclear and cytoplasmic fractions (FIG. 12B&C) does not preclude the possibility that it is recruited by the EJC. In fact, TAP could be only loosely associated with the EJC, making it susceptible to removal by RNase treatment. However, it is also possible that some TAP is recruited by the EJC and the majority is recruited by non-EJC mRNA binding proteins.

f) Discussion

273. This study aimed to characterize proteins associated with CBP80-bound and eIF4E-bound RNA in mammalian cells before and after export from the nucleus to the cytoplasm. Even though both CBP80 and eIF4E are present in nuclei (Ohno et al, 1990; Izaurralde et al., 1992; Lejbkowicz et al., 1992; Dostie et al., 2000) and eIF4E is detected in association with mRNA that co-purifies with nuclei (FIG. 8), only CBP80 is readily detected in association with intron-containing pre-mRNA (FIG. 9). The failure to detect appreciable amounts of eIF4E-bound pre-mRNA is consistent with data demonstrating that immunodepletion of CBP80 from HeLa-cell extracts strongly reduces the efficiency of pre-mRNA splicing (Izaurralde et al., 1994). It is also consistent with the detection of CBP80 but not eIF4E in association with the CTD (FIG. 10). It was found that CBP80 and not eIF4E co-immunopurifies with components of the EJC (FIG. 11). In verification that experimental conditions allowed for the isolation of mRNP and, therefore, a characterization of its structure, it was demonstrated that immunopurification of components of the EJC using anti-CBP80 antibody was sensitive to RNase whereas immunopurification of these components using anti-Upf3/3X antibody was not (FIG. 12). This result is expected of cap-bound CBP80 and distally bound components of the EJC, the latter of which recruits Upf3/3X. Our findings are consistent with data demonstrating that either Flag-Upf3 or Flag-Upf3X expressed transiently in 293T cells immunopurifies Y14 and REF/Aly independently of RNA (Kim et al., 2001b), RNPS 1, SRm160, and Upf2 were not tested and, in contrast to our results, only Flag-Upf3 immunopurified TAP in a manner that was partially dependent on RNA. eIF4E binds RNA after splicing and concomitant with or after removal of the EJC.

274. Given that CBP80-bound mRNA is the substrate for NMD and the NMD of some mRNA takes place in the cytoplasm, the replacement of CBP80 by eIF4E on mRNA subject to cytoplasmic NMD must take place in the cytoplasm at least some of the time (Ishigali et al., 2001; FIG. 13). However, the presence of eIF4E-bound mRNA in the nuclear fraction (FIG. 8) indicates that the replacement of CBP80 by eIF4E on mRNA also takes place before mRNA release into the cytoplasm at least some of the time (FIG. 13). In fact, replacement before release might typify mRNAs subject to nucleus-associated NMD.

g) Material and methods

(1) Cell Culture and Transfections

275. Monkey kidney Cos-7 were cultured and, where indicated, transfected as previously described (Ishigaki et al., 2001).

(2) Cell Fractionation, Lysis and Immunopurifications

276. Whole cells or nuclear and cytoplasmic fractions of cells (3-4×10⁷) were processed as previously described (Ishigaki et al., 2001). For all immunopurifications except those using anti-CTD antibody, samples were first cleared by incubation with end-over-end rotation in the presence of 50 μl of protein A-agarose beads (Boehringer Mannheim) in NET-2 buffer and, where specified, 1 mM PMSF (Sigma), 2 mM benzamidine (Sigma) and 100 U of RNase inhibitor (Promega) for 30 min at 4° C. followed by centrifugation at 10,000×g. Cleared samples were then rotated in the presence of either normal rabbit antiserum (NRS; GIBCO/BRL) or mouse IgG (Sigma), anti-CBP80 antibody (Izaurralde et al., 1994), anti-eIF4E antibody (Morley and McKendrick, 1997; Santa Cruz Biotechnology) or anti-hUpf3/3X antibody (Ishigaki et al, 2001) for 90 min at 4° C., after which 2 mg of yeast RNA (Sigma) and protein A-agarose beads were added. The incubation was continued for another 60 min at 4° C. The beads were washed five-six times with NET-2 buffer, suspended in 50 μl of SDS sample buffer (Ishigaki et al., 2001) that either did or, for the analysis of RNPS1 and Upf3 that co-migrated with antibody, did not contain 12% 2-mercaptoethanol, and split into two portions. In some experiments, RNase A (10 μg; Sigma) or, as a control, BSA (10 μg; New England Biolabs, Beverly, Mass.) was added to the beads and incubated for 30 min at 37° C. before elution using SDS sample buffer. One portion (six-tenths) was used as a source of protein, which was analyzed by SDS-PAGE and western blotting. The other portion (four-tenths) was extracted with phenol, chloroform and isoamyl alcohol, and precipitated using ethanol. Precipitates were dissolved in 20 μl of RQ1 DNase Buffer (Promega), treated with 10 U of DNase I (Promega) for 30 min at 37° C., extracted, precipitated, dissolved in 20 μl of water, and used as a source of RNA for RT-PCR (Sun et al. 1998).

277. For immunopurifications using anti-CTD antibody and, as a control, NRS, the concentration of NaCl in NET-2 buffer was decreased from 300 mM to 50 mM.

(3) Western Blotting

278. Protein in immunopurifications (1-5 μL) was electrophoresed in 7.5% (for SMG1/ATX), 10% (for CBP80, Upf2, SRm160, TAP, CTD), or 12% (for eIF4E, Y14, REF, DEK, Upf3X, RNPS1) polyacrylamide. Protein was transferred to Hybond ECL nitrocellulose (Amersham), and probed with antibody against CBP80 (a gift from E. Izaurralde), eIF4E (a gift from S. Morley), Upf3/3X (Serin et al., 2001), Upf2 (Serin et al., 2001), RNPS1 (a gift from A. Mayeda), Y14 (a gift from G. Dreyfuss), SRm160 (a gift from B. J. Blencowe), DEK (a gift from G. Grosveld), REF/Aly (a gift from E. Izaurralde), TAP (a gift from E. Izaurralde), SMG1/ATX (a gift from R. T. Abraham) or CTD (a gift from D. Bentley). In the case of cellular fractionations, nuclear fractions were deemed free of cytoplasm by that absence of detectable reactivity with antibody against eIF4A (a gift from S. Morely; Lejbkowicz et al., 1992) and anti-PLC-γ (a gift of R. T. Abraham). Reactivity to each primary antibody was detected using a 1:5000 dilution of anti-rabbit Ig, horse radish peroxidase-linked whole antibody (Amersham; to detect CBP80, eIF4E, RNPS1, Y14, REF/Aly, TAP, Upf2, Upf3X, SMG1/ATX and CTD); anti-mouse Ig, horse-radish peroxidase-linked whole antibody (Amersham; to detect eIF4E); or anti-mouse IgM peroxidase conjugate (Sigma; to detect SRm160). Reactivity of the secondary antibody was visualized by SuperSignal West Pico or West Femto Solution (Pierce).

(4) RT-PCR

279. Gl or MUP mRNAs in immunopurifications (10 μl) were analyzed by RT-PCR as described (Iskigaki et al., 2001). Alternatively, Gl pre-mRNA was amplified using primers 5′ GCCTATTGGTCTATTTTCCC 3′ (SEQ ID NO: 1) (sense) and 540 CCTGAAGTTCTCAGGATC 3′ (SEQ ID NO: 2) (antisense). To assay for RNase activity, endogenous Cos-cell β-actin mRNA was amplified using 5′ ATCTGGCACCACACCTTCTACAATGAGCTGCG 3′ (SEQ ID NO: 3) (sense) and 5′ CGTCATACTCCTGCTTGCTGATCCACATCTGC 3′ (SEQ ID NO: 4) (antisense). Endogenous Cos-cell SV40 T antigen (ag) pre-mRNA and mRNA were amplified using primers 5′ TGCAAGGAGTTTCATCCTG 3′ (SEQ ID NO: 5) (sense) and respectively, 5′ AGAATCAGTAGTTTAACACAC 3′ (SEQ ID NO: 6) and 5′ TGAGCATAGTTATTAATAGCAG 3′ (SEQ ID NO: 7) (antisense), in which case the annealing reaction was performed at 50 ° C. for 40 s for a total of 23 cycles. The simultaneous analysis of serial dilutions of RNA ensured that RT-PCR was quantitative. RT-PCR products were quantitated by PhosphorImaging (Molecular Dynamics).

### 3. Example 3

a) Characterization of hSMG5/7a cDNA and Encoded Protein

280. The TBLASTN algorithm was used to analyze both the human genome and human cDNA (NR) databases, resulting in the identification of a cDNA (AB018275) encoding a protein having similarity to CeSMG5 and CeSMG7. In the absence of finding proteins more closely related to either CeSMG5 or CeSMG7, this protein was named human (h) SmgS5/7a. hSmg5/a consists of 1419 amino acids and has a predicted molecular weight of 163 kD and a pI of 7.5. According to analysis in silico, hSmg5/7a contains a putative PIN domain (Clissold and Ponting 2000) and three putative nuclear localization sequences (PSORT; FIG. 14A). The hSMG5/7a gene maps to the extremity of the small arm of chromosome 17 and contains 19 exons (FIG. 14B, which shows exons within cDNA). Two Drosophila melanogaster (Dm) cDNAs, CG8954 and CG6369, were also found to encode protein-containing domains related to both CeSMG5 and CeSMG7 by using the TBLASTN algorithm to analyze the D. melanogaster genome. Protein encoded by DmCG8954 cDNA consists of 1177 amino acids and has a predicted molecular weight of 136 kD. Protein encoded by DmCG6369 cDNA consists of 949 amino acids and has a predicted molecular weight of 68 kD.

281. Amino acids 721-816 of hSmg5/7a can be aligned to amino acids 136-214 of CeSMG7 and amino acids 279-375 of DmCG6369, and each region of each protein conserved region (C) 1 has been designated (FIG. 15). Additionally, amino acids 1241-1276 of hSmg5/7a can be aligned to amino acids 422-464 of CeSMG5, amino acids 765-815 of DmCG6369, and amino acids 1008-1055 of DmCG8954, and each region of each protein C2 has been designated (FIG. 15). Furthermore, amino acids 766-825 of hSmg5/7a and amino acids 285-358 of DmCG6369 contain regions similar to amino acids 130-163 and 164-197 of CeSMG7, both of which contain probable tetratricopeptide repeats (TPRs; Cali et al. 1999). TPRs form pairs of amphipathic α-helices that generally mediate protein-protein interactions (Das et al. 1998). Also, the putative PIN domain within hSmg5/7a amino acids 1246-1397 is conserved within amino acids 426543 of CeSMG5 and has been proposed to manifest RNase activity (Clissold and Ponting 2000).

b) hSmg5/7a Purifies with PP2Ac, Upf1, Upf2, Upf3X, and Smg1

282. Initially, immunopurifications were performed to identify proteins that interact either directly or indirectly with hSmg5/7a. To this end, pCI-neo-Flag-hSMG5/7a, a vector that expresses Flag-tagged hSmg5/7a, was transiently introduced into monkey kidney Cos cells. Two days later, total-cell protein was immunopurified by using anti-Flag antibody or, as a control for nonspecific immunopurification, anti-HA antibody. Subsequently, the presence of FlaghSmg5/7a, as well as other proteins, was tested by using Western blotting.

283. Flag-hSmg5/7a was detected with anti-Flag antibody when protein was immunopurified with anti-Flag antibody but not anti-HA antibody (FIG. 16A), demonstrating the specificity of immunopurification. Because C. elegans mutants that harbor defective CeSMG5 or CeSMG7 alleles are characterized by an abnormally high level of a phosphorylated isoform of CeSMG2 (Page et al. 1999), it is reasonable to think that CeSMG5 and CeSMG7 target a phosphatase to CeSMG2. Despite the limited sequence similarity of hSmg5/7a to either CeSMG5 or CeSMG7, the 36-kD catalytic subunit (c) of PP2A was detected in the anti-Flag antibody immunoprecipitated but not the anti-HA immunoprecipitated (FIG. 16A). Upf1, Upf2, and Upf3X also were detected in the anti-Flag antibody immunoprecipitated but not the anti-HA immunoprecipitated (FIG. 16A; Upf3 was too low in abundance to be detectable, as evidenced below). The presence of both eRFs was next assayed because (1) PP2A interacts with eRF1 (Andjelkovic et al. 1996); (2) S. cerevisiae Upf1 interacts with eRF1 and eRF3 (Czaplinski et al. 1998); and (3) S. cerevisiae Upf2 and Upf3 interact with eRF3 (Wang et al. 2001). However, neither eRF1 nor eRF3 was detected in the anti-Flag antibody immunoprecipitated (FIG. 16A; see below). To assay for the possibility that Smg1 associates with Flag-hSmg5/7a, it was necessary to analyze for exogenously expressed HAtagged hSmg1 rather than endogenous Smg1 because the HA antibody is more sensitive than the available anti-Smg1 antibody. To this end, Cos cells were cotransfected with pCI-neo-Flag-hSMG5/7a and pCDNA-HA-hSMG1. Results indicate that both Flag-hSmg5/7a and HA-hSmg1 were immunopurified with anti-Flag antibody but not mouse IgG (FIG. 16A), indicating that hSmg5/7a and hSmg1 interact.

c) Microcystin Purifies Flag-hSmg5M7a, Upf1, Upf2, Upf3, Upf3X, eRF1, and eR-F3

284. Microcystin (MC)-Sepharose is often used to purify protein phosphatases and associated proteins (Moorhead et al. 1999). As an additional means to evaluate interactions of hSmg5/7a with other proteins, Flag-hSmg5/7a was transiently expressed in Cos cells, and total-cell protein was purified using MC-Sepharose and, as a control, unconjugated Sepharose. PP2Ac along with Flag-hSmg5/7a, Upf1, Upf2, Upf3, Upf3X, eRF1, and eRF3 were purified by using MC-Sepharose but not Sepharose alone (FIG. 16B). The inability to detect eRF1 or eRF3 in the immunopurification of Flag-hSmg5/7a with anti-Flag antibody (FIG. 16A) indicates that neither release factor interacts directly with hSmg5/7a.

d) Overexpression of hSmg5/7a Reduces the Phosphorylation of hUpf1

285. The finding that Flag-hSmg5/7a copurifies with PP2Ac indicates that it could function in Upf1 dephosphorylation analogously to the way CeSMG5 and CeSMG7 function in CeSMG2 dephosphorylation. To examine this possibility, pCI-neo-Flag-hSMG5/7a or, as a control, pCI-neo was transiently introduced into HEK293T cells together with pCIneo-Flag-hUPF1 (Pal et al. 2001). Two days later, total-cell protein was purified, and the ratio of Flag-hSmg5/7a to Flag-hUpf1 was determined by using Western blot analysis and anti-Flag antibody to be 1:10 (FIG. 17A). Flag-hUpf1 (together with Flag-hSmg5/7a) was then immunopurified with anti-Flag antibody, and the effect of Flag-hSmg5/7a on the phosphorylation status of Flag-hUpf1 was analyzed by using two-dimensional gel electrophoresis and Western blotting using anti-Flag antibody (Pal et al. 2001). When pCI-neo was expressed, Flag-hUpf1 was detected as multiple spots, each of which is potentially a different phosphorylated isoform (FIG. 17B; Flag-hSmg5/7a was not detected under the conditions used), as expected from previous results (Pal et al. 2001). Exposure to λ-phosphatase (λ-PPase) reduced or eliminated the more acidic isoforms, consistent with the isoforms being phosphoproteins. Notably, the degree of Flag-hUpf1 phosphorylation was also significantly reduced when pCI-neo-Flag-hSMG5/7a was expressed (FIG. 17B). Thus, hSmg5/7a functions in hUpf1 dephosphorylation.

e) Overexpression of hSmg5/7a does not Reduce the Phosphorylation of hUpf2

286. Just as mammalian Upf1 is a phosphoprotein, mammalian Upf2 could be a phosphoprotein. To test this possibility and simultaneously assay for effects of overexpressing hSmg5/7a, T7-tagged hUpf2 was transiently produced in HEK293T cells together with pCI-neo or pCI-neo-Flag-hSMG5/7a. The expression of T7-hUpf2 and Flag-hSmg5/7a was confirmed by using Western blotting with, respectively, anti-T7 antibody and anti-Flag antibody (FIG. 18A). However, the ratio of the two proteins was undeterminable because the reactivity of each antibody is undoubtedly different. T7-hUpf2 was then immunopurified by using anti-T7 antibody either with or without exposure to λ-PPase. Two-dimensional gel electrophoresis and Western blotting using anti-T7 antibody revealed that, like Flag-hUpf1, T7-hUpf2 was detected as multiple spots when pCI-neo was expressed (FIG. 18B; Flag-hSmg5/7a did not enter the gel under the conditions used). At least the majority of these spots correspond to phosphoprotein as evidenced by their reduction or elimination on exposure to λ-PPase (FIG. 18B). Therefore, T7-hUpf2 is a phosphoprotein. However, the degree of T7-hUpf2 phosphorylation was unaffected when pCI-neo-FlaghSMG5/7a was expressed (FIG. 18B), indicating that FlaghSmg5/7a does not target T7-hUpf2 for dephosphorylation.

f) Flag-hSmg5/7a is Located Primarily in the Cytoplasmic Fraction of HEK293T Cells

287. Insight into protein function often derives from information on where the protein localizes in cells. Near-normal levels of Flag-hSmg5/7a can localize as does endogenous hSmg5/7a. Therefore, HEK293T cells were transiently transfected with pCI-neo-Flag-hSMG5/7a so that the level of Flag-hSMG5/7a RNA was only 1.6-fold higher than the level of endogenous SMG5/7a RNA (FIG. 19A). Nuclear and cytoplasmic fractions were generated under conditions in which PLC-γ was detected by Western blotting primarily in the cytoplasmic fraction (FIG. 19B).

288. Flag-hSmg5/7a was detected primarily in the cytoplasmic fraction (FIG. 19B). eRF1 and eRF3 were also detected primarily but not exclusively in the cytoplasmic fraction (FIG. 19B), consistent with the recent finding that a fraction of eRF3 localizes to transcriptionally active loci, as well as the nucleolus (Brogna et al. 2002). For reasons not understood, Upf1 was detected primarily in the nuclear fraction (FIG. 19B). This result is surprising because results of in situ hybridizations indicate that Upf1 endogenous to HeLa cells, and Flag-hUpf1 transiently expressed in HeLa cells are mostly cytoplasmic (Lykke-Andersen et al. 2000; Serin et al. 2001). However, recent studies using immunofluorescence and confocal microscopy have demonstrated that the endogenous Upf1 of HeLa cells accumulates in the nuclei when CRM1-mediated nuclear export is inhibited by using leptomycin B (Mendell et al. 2002). Upf2 was detected primarily but not exclusively in the cytoplasmic fraction (FIG. 19B). Until now, immunofluorescence has demonstrated that Upf2 endogenous to HeLa cells, and T7-hUpf2 transiently expressed in HeLa cells are detected essentially exclusively in the cytoplasm, concentrated around the cytoplasmic side of the nuclear envelope (Lykke-Andersen et al. 2000; Serin et al. 2001), and HA-hUpf2 (also called RENT2) has been deemed to be exclusively cytoplasmic by using immunofluorescence coupled with confocal microscopy (Mendell et al. 2000).

### Discussion

289. The present study provides the first description of hSmg5/7a (FIG. 14A; SEQ ID NO: 37 and SEQ ID NO: 38), which has limited and localized identity and similarity to both CeSMG5 and CeSMG7 (FIG. 15). It was show that hSmg5/7a functions in the dephosphorylation of Upf1, as would be predicted if it were a functional ortholog to the C. elegans proteins (FIG. 17). Additionally, it was shown that Upf2 is a phosphoprotein but is not subject to hSmg5/7a-mediated dephosphorylation (FIG. 18).

290. Consistent with a role in Upf protein dephosphorylation, hSmg5/7a interacts with PP2Ac (FIG. 16). The PP2As are a large and diverse family of multimeric serine/threonine phosphatases that function to regulate many cellular processes in eukaryotes (Millward et al. 1999; Janssens and Goris 2001). PP2A contains two subunits in addition to the catalytic (C) subunit: a structural or scaffolding (A) subunit and a regulatory (B) subunit, the latter of which can influence phosphatase activity, substrate specificity, and subcellular location. Holoenzyme interacting proteins include substrates. Therefore, the finding that hSmg5/7a immunopurified with Upf proteins, coupled with the finding that overexpression of hSmg5/7a caused Upf1 protein dephosphorylation, indicates that hSmg5/7a delivers PP2A to Upf1 similarly to the way that eRF1 has been proposed to deliver PP2A to polysomes (Lechward et al. 1999). Along similar lines, co-immunopurification of hSmg5/7a and the PIK-related protein kinase hSmg1 (FIG. 16) reflects that both effectors target Upf1. Additionally, hSmg5/7a can target hsmg1, in keeping with the observation that the majority of PP2A substrates identified to date are protein kinases (Lechward et al. 1999). Lastly, hSmg5/7a is itself can be a substrate of PP2A.

291. eRF1 and eRF3 were not detected in the immunopurification of Flag-hSmg5/7a using anti-Flag antibody (FIG. 16A). In S. cerevisiae, both eRF1 and eRF3 interact with Upf1 (Wang et al. 2001). It has been proposed that dissociation of eRF1 from eRF3 allows binding of Upf2 and Upf3 to a putative surveillance complex through interactions with eRF3 (Wang et al. 2001). In fact, the dissociation of eRF3 from Upf1 activates the Upf1 ATPase activity (Wang et al. 2001). If Upf1 interacts with eRF1 and eRF3 in mammalian cells, then our inability to detect either release factor in association with Flag-hSmg5/7a indicates that hSmg5/7a binds to Upf1 after eRF1 and eRF3 dissociate from Upf1.

292. Flag-hSmg5/7a is mostly cytoplasmic in HEK293T cells when produced from a transiently introduced plasmid that generates Flag-hSMG5/7a RNA at only 1.6-fold the level of endogenous hSMG5/7a RNA (FIG. 19). Notably, this finding does not preclude the possibility that Flag-hSmg5/7a can be imported into nuclei, as would be understood from a computerbased analysis demonstrating the presence of three nuclear localization sequences (FIG. 14A). In fact, Flag-hSmg5/7a was mostly nuclear in Cos cells, but under conditions in which the exact level of expression relative to endogenous hSmg5/7a could not be accessed because primer pairs used to amplify hSMG5/7a transcripts did not cross-react with Cos-cell RNA.

293. A database search revealed two other human proteins that are similar to CeSMG5 and CeSMG7 (FIG. 20). These proteins are called hSmg5/7b (KIAA1089) and hSmg5/7c (KIAA0250). The hSMG5/7b gene maps to chromosome 1 and encodes a protein of 1016 amino acids with a predicted molecular weight of 114 kD and a putative PIN domain that spans amino acids 873-972. The hSMG5/7c gene also maps to chromosome 1 and encodes a protein of at least 1122 amino acids (to date, only a partial sequence is available). An interspecies comparative analysis revealed that amino acids 853-924 of hSmg5/7b can be aligned to amino acids 425-480 of CeSMG5 and amino acids 1009-1081 of DmCG8954 (FIG. 20A). Amino acids 173-257 of hSmg5/7b can be aligned to amino acids 130-208 of CeSMG7, amino acids 286-370 of DmCG6369, and amino acids 211-298 of DmCG8954 (FIG. 7A). Additionally, amino acids 186-257 of hSmg5/7c can be aligned to amino acids 133-208 of CeSMG7, amino acids 288-361 of DmCG6369, and amino acids 213-299 of DmCG8954 (FIG. 7B). An intraspecies comparative analysis revealed that amino acids 636-823 of hSmg5/7a, 65-268 of hSmg5/7b, and 84-274 of hSmg5/7c can be aligned with 21% identity and 45% similarity between hSmg5/7a and hSmg5/7b, 25% identity and 47% similarity between hSmg5/7b and hSmg5/7c, and 28% identity and 48% similarity between hSmg5/7a and hSmg5/7c. However, we were unable to identify a conserved functional motif within these regions.

294. The presence of human proteins that contain regions related to both CeSMG5 and CeSMG7 indicates that the C. elegans proteins can function as a heterodimer. In fact, CeSMG5 interacts with CeSMG7 (Cali et al. 1999), and the interaction is direct (A. Grimson and P. Anderson, pers. comm.). The finding that mutation of either C. elegans protein abolishes NMD (Pulak and Anderson 1993) and prevents the dephosphorylation of CeSMG2 (Page et al. 1999) reinforces the idea of cooperative function in NMD.

### Materials and Methods

(1) Plasmid Constructions

295. pCI-neo-Flag-hSMG5/7a was generated in two steps. First, the 6-kbp NotI-NotI fragment containing the fill-length coding region of hSmg5/7a was inserted in the NotI site of pCI-neo (Invitrogen, Carlsbad, Calif.). Second, DNA encoding the Flag epitope was PCR-generated by using Vent polymerase, pBS-hSMG5/7a, and primers 5′-GCTAGCTCGAGACCGGTGCCACCATGGACTACAAAGACGATGACGA CAAGGCGGAAGGGCTGGAGCGTGTGCGGATC-3′ (SEQ ID NO: 8) (sense, where the NheI site is italicized, and the Flag coding region is underlined) and 5′-TTTAAACCCGGCCTGCGGGGCCAGAGTAGCCAGGATCCCGCGC-3′ (SEQ ID NO: 9) (antisense, where the SfiI site is italicized). The PCR product was digested with NheI and SfiI and inserted into the NheI and SfiI sites of the plasmid generated in the first step.

(2) Cell Culture and Transfection

296. Monkey kidney Cos-7 and HEK293T cells were cultured in DMEM (GEBCO BRL) with 10% fetal calf serum. Cells were transfected with plasmids by using LipofectAMINE 2000 (Life Technologies, Carlsbad, Calif.).

(3) Cell Fractionation, Lysis, and Immunopurification or Microcystin Binding

297. Cos cells (6×10⁷ to 7×10⁷) were lysed, or 293T cells (3×10⁶ to 4×10⁶) were fractioned into nuclei and cytoplasm, and protein complexes were immunopurified essentially following the method of Ishigaki et al. (2001). For immunopurification, briefly, Cos-cell lysates were cleared using protein G-agarose beads (Boehringer Mannheim) or Sepharose beads (Sigma) and then incubated in the presence of mouse IgG (Sigma), anti-HA antibody (Roche, Basel, Switzerland), or anti-Flag antibody (Sigma) for 90 min at 4° C. Subsequently, protein G-agarose beads and 2 mg of yeast tRNA (Sigma) or microcystin-conjugated Sepharose beads (Upstate Technology) were added, and the incubation was continued for an additional 60 min or overnight at 4° C. The beads were washed five times with NET-2 buffer or lysis buffer (10 mM HEPES at pH 7.9, 10 mM KCl, 0.1 mM EDTA, 0.1 mM EGTA, 1 mM DTT, 0.1% NP-40, 1 mM PMSF, and 2 mM benzamidine) and suspended in 50 μL of SDS sample buffer (Ishigaki et al. 2001). Immunopurified material was then analyzed by using Western blotting.

(4) Western Blotting

298. Immunopurified proteins or proteins in nuclear and cytoplasmic fractions were electrophoresed in polyacrylamide (6% for FlaghSmg5/7a, HA-Smg1, Upf1, Upf2 or PLC-γ; 10% for PP2Ac, Upf3, Upf3X, eRF1, or eRF3). Proteins were transferred to Hybond ECL nitrocellulose (Amersham) and probed with antibody against Flag, HA, PP2Ac (BD Biosciences), one of the Upf proteins (Ishigaki et al. 2001; Serin et al. 2001), eRF1 or eRF3 (gifts from B. Hemmings, The Friedrich Miescher-Institut, Basel, Switzerland), PLC-γ (a gift of R. T. Abraham, The Burnham-Institute, San Diego, Calif.), or T7 (Novagen). Reactivity to each antibody was detected by using a 1:5000 dilution of horseradish peroxidase-conjugated donkey anti-rabbit antibody (Amersham; for Upfs, eRFs, or PLC-γ) or horseradish peroxidase-conjugated sheep antimouse antibody (Amersham; for Flag-hSmg5/7a, HA-Smg1, PP2Ac, or T7). Reactivity of the secondary antibody was visualized by using SuperSignal West Pico or Femto solution (Pierce).

(5) Two-Dimensional Gel Analysis

299. The two-dimensional analysis of proteins was performed essentially according to the method of Pal et al. (2001). HEK293T cells (3×10⁶ to 4×10⁶ for the analysis of Flag-hUpf1, or 2×10⁷ for the analysis of T7-hUpf2) were lysed in extraction buffer (Pal et al. 2001) in the presence or absence of 0.25 mM Na-o-vanadate, 10 mM NaF, and 10 nM okadaic acid. Lysates were incubated with anti-Flag or anti-T7 antibody for 2 h at 4° C. and, subsequently, for 2 h with protein G-agarose. The beads were washed once with λ-phosphatase buffer (New England BioLabs) and incubated with or without 400 U of λ-phosphatase (New England BioLabs) for 30 min at 30° C. The beads were then washed once with extraction buffer, and immunopurified material was eluted in sample buffer (0.1 M Tris-HCl at pH 6.8, 2% Triton X-100, 20% glycerol, 10% 2-mercaptoethanol). For the first dimension, isoelectric focusing was performed by using Bio-lytes (pH 3 to 10 and pH 5 to 7 for Flag-hUpf1; pH 4 to 6 for T7-hUpf2). For the second dimension, separation according to molecular mass was performed by electrophoresis in 6% polyacrylamide. Proteins were transferred to Hybond ECL nitrocellulose and probed with anti-Flag antibody conjugated to peroxidase or anti-T7 antibody. Reactivity of anti-T7 antibody was detected by using horseradish peroxidase-conjugated sheep anti-mouse antibody. Reactivity of the primary or secondary antibody was visualized using SuperSignal West Femto solution.

(6) RT-PCR

300. TPI and hSMG5/7a mRNAs were analyzed by RT-PCR as described (Iskigaki et al. 2001). TPI mRNA was amplified using primers 5′-TGACCTTCAGCGCCTCGG-3′ (SEQ ID NO: 10) (sense) and 5′-CTCCGAGTCCCTCTGCC-3′ (SEQ ID NO: 11) (antisense). hSMG5/7a mRNA was amplified using primers 5′-GGCAAAGGCTCTGAGAAGC-3′ (SEQ ID NO: 12) (sense) and 5′-CCGAGGTCCCAAAGGCG-3′ (SEQ ID NO: 13) (antisense). The simultaneous analysis of serial dilutions of RNA ensured that RT-PCR was quantitative. RT-PCR products were quantitated by PhosphorImaging (Molecular Dynamics).

### 4. Example 4

a) Down-regulating the Dcp2 decapping protein of the 5′-to-3′ mRNA decay pathway or either the PM/Scl100 exosomal component or PARN of the 3′-to-5′ mRNA decay pathway abrogates both nucleus-associated and cytoplasmic NMD

301. It has been demonstrated that NMD targets newly synthesized mRNA that is bound by cap binding protein (CBP) 80, which is primarily nuclear but also shuttles to the cytoplasm (Ishigali et al., 2001; Lejeune et al., 2002). Most mRNAs are subject to nucleus-associated NMD, i.e., they undergo decay prior to release into the cytoplasm (either in the nucleoplasm or during export to the cytoplasm), while a minority of mRNAs is subject to cytoplasmic NMD (for review, see Hentze and Kulozik, 1999; Maquat, 2000; Wagner and Lykke-Andersen, 2002).

302. In theory, NMD could be exonucleolytic, initiating at either or both mRNA ends, or endonucleolytic. Initially, a component of the 5′-to-3′ exonucleolytic pathway was assessed since this pathway had been shown to function in NMD in S. cerevisiae (Hagan et al., 1995; Muhlrad and Parker, 1994; Muhlrad and Parker, 1999). Furthermore, endonucleolytic pathways are often mRNA-specific (Schoenberg and Chemokalskaya, 1997). At the same time, a component of the 3′-to-5′ exonucleolytic decay pathway was assessed. To this end, siRNAs were generated in vivo or in vitro to down-regulate the Dcp2 decapping protein of the 5′-to-3′ decay pathway or the PM/Scl100 exosomal component of the 3′-to-5′ decay pathway. As controls, siRNAs against no particular protein were separately introduced. Effects of each siRNA were assayed in HeLa cells that had been transiently transfected with the reference plasmid phCMV-MUP, which generates transcripts encoding the mouse major urinary protein (Belgrader and Maquat, 1994), and one of two sets of test plasmids: either (i) pmCMV-Gl Norm or pmCMV-GPx1 Norm, each of which lacks a nonsense codon and generates, respectively, β-globin (Gl) or glutathione peroxidase 1 (GPx1) transcripts using the mouse cytomegalovirus promoter (Zhang et al., 1998; Moriarty et al., 1998), or (ii) pmCMV-Gl 39Ter or pmCMV-GPx1 46Ter, each of which is identical to the corresponding Norm construct except for harboring a nonsense codon at position 39 or 46, respectively, that elicits NMD (Zhang et al., 1998; Moriarty et al., 1998). Cells were harvested two days after transfection, and total-cell protein as well as nuclear and cytoplasmic RNAs were purified. Notably, while mCMV-Gl (hereafter called Gl) 39Ter mRNA is subject to nucleus-associated NMD (Zhang et al., 1998; Thermann et al., 1998), mCMV-GPx1 (hereafter called GPx1) 46Ter mRNA is subject to cytoplasmic NMD (Moriarty et al., 1998). Together, they offer the potential to determine contributions of Dcp2 and PM/Scl100 to NMD at each cellular site.

303. The efficiency of Dcp2 and PM/Scl100 down-regulation was assessed by Western blotting. The level of Upf3X was used to control for differences between samples in protein recovery. Results indicate that the most effective siRNAs reduced the level of Dcp2 or PM/Scl100 to ˜35% of normal, while Control siRNA had no effect on the level of either protein (FIG. 21A, upper). Using the level of MUP mRNA to control for variations in transfection efficiencies and RNA recovery, comparisons of the amounts of nucleus-associated Gl Norm and Gl 39Ter mRNAs or cytoplasmic GPx1 Norm and GPx1 46Ter mRNAs in the presence of Dcp2 siRNA or PM/Scl100 siRNA relative to Control siRNA demonstrated that a reduction in the level of either degradative factor increased the level of nonsense-containing mRNA three-fold to five-fold (FIG. 21A, middle and lower). Notably, down-regulation of either protein did not affect the amount of cap-dependent luciferase activity produced from a transiently introduced plasmid (FIG. 28), indicating that the increased levels of nonsense-containing mRNAs are not due to decreased rates of cellular translation. Thus, both nucleus-associated and cytoplasmic NMD involve decapping and exosome-mediated decay. Similar results were obtained using HEK 293T cells.

304. In order to corroborate that NMD involves 3′-to-5′ mRNA decay, the effect of down-regulating PARN was also examined. Down-regulating PARN to 15% of the normal level (FIG. 21B, upper) increased the levels of nucleus-associated Gl 39Ter mRNA and cytoplasmic GPx1 46Ter mRNA 3-fold and 2-fold, respectively (FIG. 21B, middle and lower). Therefore, both nucleus-associated and cytoplasmic NMD involve deadenylation and, subsequently, 3′-to-5′ exonucleolytic decay.

305. In order to demonstrate that the increased level of nonsense-containing mRNA resulting when either Dcp2 or PM/Scl100 is down-regulated is due to an increase in mRNA half-life rather than an indirect effect of inhibiting a cellular process other than NMD, pfos-Gl Norm or pfos-Gl 39Ter were generated so that Gl gene transcription is transiently inducible upon the addition of serum to serum-deprived cells (see, e.g., Belgrader et al., 1994). The effect of Dcp2 siRNA, PM/Scl100 siRNA or Control siRNA on the NMD of fos-Gl 39Ter mRNA was assayed by transiently transfecting L cells, which effectively synchronize in G0 upon serum starvation, with the serum-insensitive pmCMV-MUP reference plasmid and either the pfos-Gl Norm or pfos-Gl 39Ter test plasmid. Briefly, cells were washed 12 hr after transfection and incubated in medium lacking serum. After an additional 24 hr, cells were incubated in medium containing 15% serum and harvested at subsequent intervals for the analysis of nuclear RNA as well as cytoplasmic protein.

306. The results of Western blotting demonstrate that the level of Dcp2 or PM/Scl100 was reduced to <10% or ˜40% of normal by the respective siRNA at time 0 (i.e., the time of serum addition) and was unaffected by Control siRNA (FIGS. 22A and 22C). Notably, differences between the extent to which Dcp2 or PM/Scl100 were down-regulated in these experiments, which employed L cells, and in experiments employing HeLa cells (FIG. 21) undoubtedly reflect the use of different (i.e., species-specific) siRNAs.

307. Using the level of MUP mRNA to control for variations in transfection efficiencies and RNA recovery, results obtained using RT-PCR and nucleus-associated RNA indicate that the level of fos-Gl 39Ter mRNA in the presence of Control siRNA at time 0 was 24% the level of fos-Gl Norm mRNA (FIG. 22B), consistent with our previous finding using a triose phosphate isomerase (TPI) transcript that substitution of a serum-insensitive promoter with the fos promoter does not alter the cellular site of NMD (Belgrader et al., 1994; see below). In the presence of Control siRNA, fos-Gl Norm and 39Ter mRNA production was maximally induced by 30 min after serum addition, and the NMD of fos-Gl 39Ter mRNA was evident thereafter (FIG. 22B, upper). Down-regulation of Dcp2 or PM/Scl100 increased the level of fos-Gl 39Ter mRNA at time 0 to, respectively, 60% or 90% of the level obtained in the presence of Control siRNA (FIG. 22B). Therefore, consistent with studies using HeLa cells (FIG. 21), down-regulation of either degradative protein abrogated NMD three-fold to four-fold. Notably, differences in the relative levels of Norm and 39Ter mRNAs and the efficacies with which down-regulated protein abrogated NMD between HeLa and L cells can reflect cell-type differences. Importantly, down-regulation of Dcp2 or PM/Scl100 also decreased the rate of NMD without significantly affecting the metabolism of fos-Gl Norm mRNA, as evidenced at times after 30 min of serum addition (FIGS. 22B and 22C). These results, which employed transiently synthesized mRNA, support our conclusion obtained using steady-state mRNA that NMD involves decapping and exosome-mediated decay.

b) PM/Scl100 is detected in both the nucleus and cytoplasm of HeLa and 293T cells using indirect immunofluorescence and confocal microscopy

308. Consistent with a role in both nucleus-associated and cytoplasmic NMD (FIGS. 21 and 22), Dcp2 is nuclear and cytoplasmic (Chen et al., 2001; Lykke-Andersen, 2002; Van Dijk et al., 2002; Wang et al., 2002). The finding that PM/Scl100 is also involved in both nucleus-associated and cytoplasmic NMD (FIGS. 21 and 22) indicates that this exosomal component also functions at both cellular locations. While data indicate that Rrp6p, the PM/Scl100 orthologue in S. cerevisiae, is confined to the nucleus in S. cerevisiae (Allmang et al., 1999), there is some controversy over whether or not the same is true for PM/Scl100 in mammalian cells. Using HeLa cells, one study reported that PM/Scl100 was detected in nuclear but not cytoplasmic extract (Allmang et al., 1999). Nevertheless, two other studies identified PM/Scl100 in both nuclear and cytoplasmic extract (Brouwer et al., 2001; Chen et al., 2001). Since results deriving from biochemical fractionation can be confounded by nuclear leakage, cytoplasmic contamination of nuclei, or both, indirect immunofluorescence together with confocal microscopy were used to localize PM/Scl100 and, as a control, the Rrp4 component of the exosome within Hela and 293T cells. Notably, Rrp4 has been localized using either indirect immunofluorescence or biochemical fractionation to both nuclei and cytoplasm of S. cerevisiae and HeLa cells (Alimang et al., 1999).

309. Western blotting demonstrated that each antibody specifically reacted with a single, appropriately sized protein in HeLa-cell and 293T-cell extracts (FIG. 23A), and normal rabbit serum (NRS), which was used to control for non-specific immunofluorescence, reacted with no protein. As expected, NRS failed to generate fluorescence in the nuclei or cytoplasm of HeLa cells (FIG. 23B) or 293T cells (FIG. 23C). In contrast, PM/Scl100 was detected in both nuclei and cytoplasm of both cell types (FIGS. 23B and 23C). Within nuclei, the concentration at nucleolar might reflect the role of PM/Scl100 in rRNA maturation, as has been shown for Rrp6p in S. cerevisiae (Briggs et al., 1998). As has been previously reported (Allmang et al., 1999), Rrp4 was also detected in both nuclei and cytoplasm, with less of a nucleolar concentration than was observed for PM/Scl100 (FIGS. 23B and 23C). Thus, the cellular distribution of PM/Scl100 is compatible with a role in nucleus-associated as well as cytoplasmic NMD.

c) All three Upf NMD factors co-immunopurify with proteins involved in the 5′-to-3′ decay and 3′-to-5′ decay of mRNA: Dcp2, Rat1, Xrn1, PM/Sc100, Rrp4, Rrp41 and PARN

310. Insight into the polarity and enzymology of NMD can also derive from an assessment of factors that bind to Upf factors, which are required for NMD. In order to increase the likelihood that the assessment reflects normal physiology, endogenous cellular proteins were analyzed rather than epitope-tagged proteins produced at what are usually abnormal levels from expression vectors. Upf1, Upf2 or Upf3X were immunopurified from Cos cells using, respectively, anti-Upf1, anti-Upf2 or anti-Upf3/3X antibody under conditions that preserve mRNP (Ishigaki et al., 2001; Lejeune et al., 2002). In a control experiment performed in parallel, NRS was used in place of an anti-Upf antibody. Western blotting was then performed to assess the efficiency of each immunopurification (IP) as well as the presence of Dcp2, the putative 5′-to-3′ exoribonuclease Rat1, the proven 5′-to-3′ exoribonuclease Xrn1, PM/Scl100, the exosomal components Rrp4 and Rrp41, and PARN.

311. Consistent with the findings that NMD involves Dcp2, PM/Scl100 and PARN (FIGS. 21 and 22), each Upf factor was found to co-immunopurify with not only Dcp2, PM/Scl100 and PARN but also the Rat1 and Xrn1 exonucleases that are thought to degrade mammalian mRNAs after decapping, and the Rrp4 and Rrp41 exosomal components that function in conjunction with PM/Scl100 and after PARN (FIG. 24). The presence of each factor in each IP was shown to be specific as evidenced by (i) the failure to detect any degradative factor in the NRS IP, and (ii) the failure to detect the major cytoplasmic cap binding protein eIF4E or nuclear Flap endonuclease (Fen) 1 in any Upf IP (FIG. 24). In related experiments, all interactions in the IP using anti-Upf3/3X antibody were found to be insensitive to the addition of RNase (IPs using anti-Upf1 or anti-Upf2 antibody were not assayed), and anti-Dcp2 antibody was found to immunopurify Upf1, Upf2, Upf3X, Rat1, PM/Scl100, Rrp41 and PARN in the presence or absence of RNase. Therefore, the interactions, once formed, do not require mRNA. Notably, the integrity of the interactions was not destroyed when either Dcp2 or PM/Scl100 was down-regulated (FIG. 29), which is consistent with the conclusion that NMD involves both 5′-to-3′ and 3′-to-5′ mRNA decay.

d) The decay rate of full-length Gl Ter mRNA is comparable to the decay rate of its 5′ end and comparable if not slightly faster than the decay rate of its 3′ end

312. In order to examine the kinetics of 5′-end and 3′-end decay relative to full-length mRNA decay during NMD, the relative decay rates of fos-Gl Norm mRNA and fos-Gl 39Ter mRNA were assessed in mouse L cells after the fos promoter was transiently induced by the addition of serum to serum-deprived cells as described above (FIG. 22). Results obtained using RT-PCR and nucleus-associated RNA indicate that the level of fos-Gl 39Ter mRNA at time 0 was 22% the level of fos-Gl Norm mRNA FIG. 25A). As observed previously (FIG. 22), fos-Gl Norm and 39Ter mRNA production was maximally induced by 30 min after serum addition, and the NMD of fos-Gl 39Ter mRNA was evident thereafter (FIG. 25A).

313. In order to assess the relative rates of 5′- and 3′-end loss, the levels of fos-Gl Norm and 39Ter mRNA 5′ and 3′ ends were quantitated beginning with the 30-min time point using RT-PCR, when pre-mRNA levels were sufficiently low so as not to confound measurements of mRNA. In the case of 5′-end measurements, 94 nucleotides of the 5′ UTR were amplified. In the case of 3′-end measurements, 110 nucleotides of the 3′ UTR were amplified. Results indicate that each end of nonsense-containing mRNA decreased in abundance faster than the corresponding end of nonsense-free mRNA (FIG. 25B). The findings that the amount of 39Ter mRNA 5′ ends at 90 minutes was 38% of the amount at 30 minutes (FIG. 25B), and the amount of full-length 39Ter mRNA at 90 minutes was 35% of the amount at 30 minutes (FIG. 25A) indicate that the decay of mRNA 5′ ends proceeds at essentially the same rate as the decay of full-length mRNA. A direct comparison of the relative rates of 5′- and 3′-end decay revealed that the 5′ end of fos-Gl 39Ter mRNA was degraded ˜1.5-fold faster than the 3′ end (FIG. 25C). A comparable if not slightly faster rate of 5′-end decay relative to 3′-end decay was obtained in each of three independently performed experiments (see e.g., FIG. 30). Taken together with data presented above, these results indicate that NMD initiates at both 5′ and 3′ ends.

e) Deadenylated Gl Ter mRNA is not detectable during NMD

314. Data indicate that NMD involves both 5′-to-3′ and 3′-to-5′ decay. Detectable deadenylated intermediates can be found for mRNAs in S. cerevisiae, especially when the major 5′-to-3′ decay pathway is inhibited (for review, see Tucker and Parker, 2000), as well as in mammalian cells, in the case of, e.g., mRNAs harboring an AU-rich element (for review, see van Hoof and Parker, 2002; Wilson and Brewer, 1999). Since NMD involves 3′-to-5′ decay, it might be possible to detect deadenylated intermediates. Alternatively, the rate of deadenylation could be fast enough to preclude the detection of deadenylated mRNA.

315. In order to assay for deadenylated decay intermediates, L cells were transiently transfected with the MUP reference plasmid and either the fos-Gl Norm or 39Ter test plasmid, and steps of serum addition after serum deprivation were repeated as described above (FIGS. 22 and 25). In this experiment, however, nuclear as well as cytoplasmic RNAs were prepared at different times after serum addition (as long as 120 minutes in the case of cytoplasmic RNA). Furthermore, not only were the kinetics of NMD measured using RT-PCR and nuclear RNA, but the lengths of the poly (A) tails on fos-Gl Norm and 39Ter mRNAs were compared using Northern blotting and nuclear or cytoplasmic RNA.

316. Consistent with data described above (FIGS. 22 and 25), an analysis of nuclear RNA demonstrated that the NMD of fos-Gl 39Ter mRNA was evident after 30 minutes of serum addition to serum-deprived cells using either RT-PCR (FIG. 26A) or Northern blotting (FIG. 26B; since there was too little RNA to quantitate by OD, the levels of MUP mRNA can be used to control for variations between lanes in the amounts of Gl mRNA analyzed). The results of Northern blotting also indicate that the size of newly induced nuclear fos-Gl Norm and 39Ter mRNAs (i.e, mRNAs detected at 30 minutes after serum addition and beyond) was larger than the size of steady-state (i.e., constitutively produced) mCMV-Gl Norm and 39Ter mRNAs driven by the mouse CMV promoter and isolated from total-cell RNA (FIG. 26B, A⁺). This finding is consistent with the fact that newly synthesized nuclear mRNA has a longer poly (A) tail than steady-state total-cell mRNA, which is mostly cytoplasmic mRNA. The same conclusion can be drawn for MUP mRNA (FIG. 26B). In the case of fos-Gl Norm mRNA, there was even a detectable difference in size between 0 and 30 minutes after serum addition (FIG. 26B), consistent with poly (A) shortening taking place in association with nuclei. NMD reduced the level of fos-Gl 39Ter mRNA at time 0 to below detectable so that a similar conclusion could not be drawn.

317. Notably, neither fos-Gl Norm mRNA nor fos-Gl 39Ter mRNA progressively decreased in size during times when NMD was active (FIG. 26B). Since electrophoretic conditions allowed for a distinction between poly (A)⁺ and poly (A)⁻ steady-state mCMV-Gl Norm or 39Ter mRNAs, the latter of which was generated by isolating and incubating total-cell RNA with oligo (dT) and RNase H FIG. 26B), results obtained for fos-Gl 39Ter mRNA indicate that partially or completely deadenylated species were not detectable during NMD (FIG. 26B). The failure to detect deadenylated mRNA during NMD is also supported by previous studies of serum-induced, nucleus-associated fos-TPI 189Ter mRNA (Belgrader et al., 1994), where we showed that there was no detectable shortening of the mRNA with time but, unlike now, had no indication that NMD involved 3′-end decay. The absence of detectable deadenylated mRNA, together with the data implicating roles for PARN and the exosome in NMD (FIGS. 21, 22 and 24), indicate that either NMD does not involve 3′-to-5′ decay at a level that permits the detection of deadenylated intermediates under the conditions used here or that the rate of deadenylation and subsequent 3′-to-5′ decay of the body of the transcript is sufficiently fast to preclude the detection of decay intermediates.

318. The size of cytoplasmic fos-Gl mRNA was also examined. As expected given that mRNAs that are subject to nucleus-associated NMD are immune to NMD after release into the cytoplasm (e.g., Belgrader and Maquat, 1994; Cheng and Maquat, 1993), 39Ter does not accelerate the rate of cytoplasmic deadenylation or otherwise elicit cytoplasmic mRNA decay (FIG. 26C). The decrease in poly (A) tail length that is evident for both Norm and 39Ter mRNAs beginning 60 min after serum addition represents shortening of the poly (A) tail on newly synthesized mRNA toward the length on steady-state mRNA. Consistent with this interpretation, there is no decrease in poly (A) tail length evident for MUP mRNA, which is exclusively steady-state mRNA (FIG. 26C). Therefore, NMD accelerates the 3′-to-5′ decay of nucleus-associated fos-Gl 39Ter mRNA without generating detectable deadenylated intermediates and does not accelerate the 3′-to-5′ decay of cytoplasmic fos-Gl 39Ter mRNA.

### Discussion

319. Here is shown for the first time that NMD in mammalian cells involves 5′-to-3′ as well as 3′-to-5′ mRNA decay based on the following results: (i) down-regulation of the Dcp2 decapping protein, the PM/Scl100 component of the exosome, or PARN abrogates both nucleus-associated and cytoplasm NMD (FIGS. 21 and 22), and (ii) all three Upf NMD factors co-immunopurify with Dcp2, PM/Scl100 and PARN as well as Rat1 and Xrn1, which are putative and proven 5′-to-3′ exonucleases respectively, and the Rrp4 and Rrp41 components of the exosome (FIG. 24). The decay rate of full-length fos-Gl Ter mRNA is comparable to the decay rate of its 5′ end and comparable if not slightly faster than the decay rate of its 3′ end (FIG. 25), which is consistent with data indicating that NMD initiates at both ends. The failure to detect deadenylated mRNA during NMD indicates that either 3′-to-5′ decay is sufficiently minor or deadenylation and the subsequent 3′-to-5′ decay of the mRNA body is sufficiently fast to preclude the detection of deadenylated decay intermediates (FIG. 26). Thus, NMD involves (i) 5′-to-3′ mRNA decay by engaging the Dcp2 decapping enzyme and, subsequently, Rat1 or Xrn1 5 ′-to-3′ exonucleases and (ii) 3 ′-to-5′ mRNA decay by engaging PARN and, subsequently, 3′-to-5′ exonucleases of the exosome (FIG. 27).

320. The use of siRNAs to demonstrate roles for the decapping enzyme Dcp2 and PM/Scl100 component of the exosome in both nucleus-associated and cytoplasmic NMD indicates that each activity is situated within mammalian cells so as to function at both cellular sites. While it is currently unclear if nucleus-associated NMD takes place in the nucleoplasm or in the cytoplasm during mRNA export, Dcp2 is detected in both nuclear and cytoplasmic fractions (Chen et al., 2001; Lykke-Andersen, 2002; Van Dijk et al., 2002; Wang et al., 2002), as is PM/Scl100 FIG. 23). Since Upf2 and Upf3X are detected on CBP80-bound mRNA, which is the target of NMD, but not eIF4E-bound mRNA, which is immune to NMD (Ishigaki et al., 2001; Lejeune et al., 2002), the co-IP of Upf2 and Upf3X with decay factors undoubtedly reflects decay factor function in NMD. However, the co-IP of Dcp2 and, if analogies can be made to yeast (Peltz and Jacobson, 2000), Upf1 with the same decay factors must primarily reflect decay factor function in degrading nonsense-free mRNAs, which constitute the bulk of cellular mRNAs. Notably, in HeLa cells, Dcp2 and Xrn1 have been shown to co-localize in cytoplasmic foci together with Dcp1 and Lsm1-7 proteins, which are required for 5′-to-3′ mRNA decay, indicating that these decay proteins may constitute a partially or fully assembled degradative complex (Ingelfinger et al., 2002; Van Dijk et al., 2002) that can be analogous to the cytoplasmic P bodies recently described for S. cerevisiae (Sheth and Parker, 2003). Additional evidence for complex formation derives from the finding that Upf1, Upf2, Upf3X, Dcp2, Rat1, Xrn1, PM/Scl100, Rrp41 and PARN co-immunopurify, consistent with other studies of mammalian cells as well as S. cerevisiae demonstrating that Upf1 interacts directly or indirectly with decapping proteins (Lykke-Andersen, 2002; He and Jacobson, 1995).

321. Despite NMD in mammalian cells and S. cerevisiae sharing the common feature of involving 5′-to-3′ and 3′-to-5′ decay, there are significant differences. As one difference, NMD in mammalian cells is restricted to newly synthesized mRNA and can take place either in association with nuclei or in the cytoplasm. In contrast, data indicate that NMD in S. cerevisiae targets steady-state mRNA in the cytoplasm (Maderazo et al., 2003; Zhang et al., 1997). As another difference, 5′ and 3′ nonsense codons in mammalian transcripts are generally (but not always) able to elicit NMD with comparable efficiencies (e.g., Li and Wilkinson, 1998; Maquat, 2000). In contrast, 5′ nonsense codons in S. cerevisiae are generally more efficient at eliciting NMD than 3′ nonsense codons (e.g., Hagan et al., 1995; Losson and Lacroute, 1979; Peltz et al., 1993).

f) Methods

(1) Plasmid Constructions

322. Nonsense-free (Norm) or nonsense-containing (39Ter) pfos-Gl plasmids, in which β-globin gene expression was driven by the human c-fos promoter, were generated by replacing the 4.36-kbp NcoI-EcoRI fragment of pfos-TPI (Cheng and Maquat, 1993; Belgrader et al., 1994) with the 2.03-kbp NcoI-XbaI fragment of pmCMV-Gl Norm or pmCMV-Gl 39Ter (Sun et al., 1998), respectively. Prior to ligation, the EcoRI- and XbaI-generated ends were filled using Klenow fragment.

(2) siRNA-Mediated Down-Regulation of Dcp2, PM/Scl100 or PARN

323. Small interfering (si) RNAs were synthesized either in vivo using a template that was constructed following a two-step PCR protocol (Castanotto et al., 2002) or in vitro (Xeragon) as described below.

324. For the down-regulation of Dcp2 in human cells, a PCR fragment was generated following a two-step PCR following a two-step PCR protocol (Castanotto et al., 2002). In the first step, a 300-bp PCR fragment was generated from pAV-U6+27 (a gift from D. Engelke) using primer pair 5′ ATCGAAGATCTGGATCCAAGGTCGGGCAGGAGAGGGCCT 3′ (SEQ ID NO: 14) (sense) and 5′ TACACAAAGCAATGTCCATTACATGCCACGGTGTTTCGTCCTTTCCACAAGA TATATAA (SEQ ID NO: 15) (antisense, where nucleotides complementary to Dcp2 mRNA are underlined). In the second step, a portion of the PCR product generated in the first step was amplified using the same sense primer and 5′ CGAAATCTAGAAAAAAGTGGCATGTAATGGACATTGCCTACACAAAGC 3′ (SEQ ID NO: 16) (antisense, where nucleotides corresponding to Dcp2 mRNA are underlined). For the down-regulation of PM/Scl100 in human cells, the double-stranded 5′ r(GCUGCAGCAGAACAGGCCA)d(TT) 3′ (SEQ ID NO: 17) (sense) siRNA was synthesized (Xeragon). As a control, the double-stranded 5′ r(GUACAACCCAGGAUAUGUG)d(TT) 3′ (SEQ ID NO: 18) (sense) non-specific siRNA also synthesized. For the down-regulation of PARN in human cells, a PCR fragment was generated as described above except that the antisense primers in the first and second steps were, respectively, 5′ TACACAAACAGGGCTGTTCTTCGAGATGCGGTGTTTCGTCCTTTCCACAAGA TATATAA 3′ (SEQ ID NO: 19) (where nucleotides corresponding to PARN mRNA are underlined) and 5′ CGAAATCTAGAAAAAAGCATCTCGAAGAACAGCCCTGCTACACAAACA 3′ (SEQ ID NO: 20).

325. For the down-regulation of Dcp2 in mouse cells, a PCR fragment was generated as described above except that the antisense primers in the first and second steps were, respectively, 5′ TACACAAAGCAATGTCCGTTGCATGCCACGGTGTTTCGTCCTTTCCACAAGA TATATAA 3′ (SEQ ID NO: 21) and 5′ CGAAATCTAGAAAAAAGTGGCATGCAACGGACATTGCCTACACAAAGC 3′ (SEQ ID NO: 22). For the down-regulation of PM/Sc100 in mouse cells, antisense primers in the first and second step of the two-step PCR protocol were, respectively, 5′ TACACAAAGTTCAGAGGCTGTGTCATAACGGTGTTTCGTCCTTTCCACAAGA TATATAA 3′ (SEQ ID NO: 23)and 5′ CGAAATCTAGAAAAAAGTTATGACACAGCCTCTGAACCTACACAAAGT 3′ (SEQ ID NO: 24). Control siRNA was generated similarly using 5′ TACACAAACCAAGGCACTTGTTGGCAGTCGGTGTTTCGTCCTTTCCACAAGA TATATAA 3′ (SEQ ID NO: 25) and 5′ CGAAATCTAGAAAAAAGACTGCCAACAAGTGCCTTGGCTACACAAACC 3′ (SEQ ID NO: 26) as antisense primers in the first and second steps, respectively.

(3) Analysis of NMD Using Steady-State Gl or GPx1 mRNA

326. HeLa cells were grown in Dulbecco's Modified Eagle Medium (NMEM; Invitrogen) and 10% fetal bovine serum (FBS; Ivitrogen). Cells were transfected using Oligofectamine (Invitrogen) with the specified amount of either a PCR fragment that produces siRNA in vivo, in vitro-synthesized siRNA, or no siRNA. Three days after transfection, each dish was split into four. After an additional 24 hr, cells were re-transfected with the same PCR fragment, siRNA or no siRNA as in the first transfection as well as one or more test plasmids [pmCMV-Gl (Sun et al., 1998); pmCMV-GPx1 (Moriarty et al., 1998)] and a reference plasmid [phCMV-MUP (Belgrader and Maquat, 1994)]. Cells were harvested 2 days later. Total-cell protein was isolated from half of the cells (Lejeune et al., 2002), and 20 μg was electrophoresed in 10% SDS-polyacrylamide and analyzed by Western blotting. Nuclear and cytoplasmic RNA was isolated from the other half of the cells, and GI, GPx1 MUP mRNAs were subject to RT-PCR (Ishigaki et al., 2001).

(4) Analysis of NMD Using Serum-Induced fos-Gl mRNA

327. L-M(TK⁻) cells were grown in DMEM with 10% FBS. In experiments that did not employ siRNA, cells were transiently transfected using Lipofectamine 2000 Invitrogen) with a pfos-Gl test plasmid and the phCMV-MUP reference plasmid. In experiments that employed siRNA, siRNA was introduced 3 days before and, again, concomitantly with test and reference plasmids using Oligofectamine. To induce fos-Gl gene expression, cells were washed twice 12 hr after transfection with serum-free DMEM medium and subsequently maintained in serum-free DMEM medium for 24 hr. The medium was then replaced with DMEM containing 15% FBS, and cells were harvested at specified times thereafter. Nuclear and cytoplasmic fractions were isolated. In experiments using siRNA, protein was isolated from the cytoplasmic fractions for Western blotting. In all experiments, RNA was isolated from the nuclear fraction for RT-PCR.

328. Briefly, fos-Gl mRNA was amplified using the primer pair 5′ GCAGCGAGCAACTGAGAAGC 3′ (SEQ ID NO: 27) (sense) and 5′ GGGTTTAGTGGTACTTGTGAGC 3′ (SEQ ID NO: 28) (antisense). The 5′ end of fos-Gl mRNA (94 nt) was amplified using the primer pair 5′ GACTGAGCCGATCCCGCGC 3′ (SEQ ID NO: 29) (sense) and 5′ GCAGTAACGGCAGACTTCTC 3′ (SEQ ID NO: 30) (antisense). The 3′ end of fos-Gl mRNA (110 nt) was amplified using the primer pair 5′ CCTTTCCTGCTCTTGCCTG 3′ (SEQ ID NO: 31) (sense) and 5′ GCTTTTTATTTGTCAGAAGACAG 3′ (SEQ ID NO: 32) (antisense). Full-length fos-Gl and MUP mRNAs were amplified using 19 PCR cycles (95° C. for 50 sec, 57° C. for 40 sec, 72° C. for 60 sec), and PCR products were analyzed in 5% polyacrylamide. The 5′ and 3′ ends of fos-Gl mRNA were amplified using 21 PCR cycles (95° C. for 50 sec, 57° C. for 40 sec, 72° C. for 40 sec), and PCR products were analyzed in 8% polyacrylamide. Products were quantitated using PhosphorImaging (Molecular Dynamics).

329. The polyadenylation status of fos-Gl and MUP mRNAs in nuclear and cytoplasmic fractions was analyzed by RNA blotting after electrophoresis in 6% formaldehyde-1.4% agarose gels (Shyu et al., 1991). Gl and MUP mRNAs were detected in 4 μg of nuclear or cytoplasmic RNA using a random-primed (Promega) restriction fragment: a 170-bp MscI-DraI β-Gl gene fragment (Zhang et al., 1998), and 800-bp PstI-PstI MUP gene fragment (Belgrader and Maquat, 1994). As controls, 2 μg of total-cell RNA from Cos cells transfected using calcium phosphate (Ishigaki et al., 2001) with pmCMV-Gl Norm and phCMV-MUP were analyzed in parallel after treatment with or without 0.2 μg of oligo(dT)₁₂₋₁₈ (Invitrogen) and 0.1 U of RNase H (Invitrogen) at 37° C. for 1.5 hr followed by incubation at 95° C. for 2 min. Hybridized radioactivity was detected using PhosphorImaging.

(5) Confocal Microscopy

330. HEK 293T or HeLa cells were grown on glass cover slips and fixed in 100% methanol for 10 min at −20° C. Cells were incubated with primary antibodies overnight at 4° C., washed three times with PBS, and incubated with anti-rabbit goat antibody Alexa Fluor 568 (Molecular Probe) for one hr at 37° C. Subsequently, cells were washed three times with PBS, incubated with 20 μg of 4′,6-diamidino-2-phenylindole, dihydrochloride (DAPI; Molecular Probe) for 30 sec at room temperature, rinsed with water, and analyzed using confocal microscopy (LEICA TCS-NT, Allendale, N.J.).

(6) Co-Immunopurifications

331. Immunopurifications (IPs) and Western blotting were as described previously (Lejeune et al., 2002). In those IPs that employed RNase, RNA was extracted from a fraction (⅖) of each IP (Ishigaki et al., 2001). A portion (⅕) of extracted RNA was reversed transcribed (Ishigaki et al., 2001), and β-actin mRNA was then amplified using 30 cycles of PCR (95° C. for 50 sec, 55° C. for 40 sec, and 72° C. for 60 sec) and the primer pair 5′ ATCTGGCACCACACCTTCTACAATGAGCTG 3′ (SEQ ID NO: 33) (sense), and 5′ CGTCATACTCCTGCTTGCTGATCCACATCT 3′ (SEQ ID NO: 34) (antisense). Half of each PCR reaction was electrophoresed in 1% agarose containing ethidium bromide and detected using UV in order to assess the effectiveness of RNase treatment.

(7) Luciferase Activity Assays

332. Renilla luciferase activity was measured using the Dual-Luciferase® Reporter Assay System (Promega) according to manufacturer instructions. Renilla luciferase mRNA was subject to RT-PCR (Ishigaki et al., 2001) using 5 μg, 19 PCR cycles, and the primer pair 5′ ATGACTTCGAAAGTTTAT 3′ (SEQ ID NO: 35) (sense) and 5′ TTCAGATTTGATCAACGCA 3′ (SEQ ID NO: 36) (antisense).

333. Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

334. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### G. REFERENCES

Alexiadis, V., Waldmann, T., Andersen, J., Mann, M., Knippers, R, and Gruss, C. (2000) the protein encoded by the proto-oncogene DEK changes the topology of chromatin and reduced the efficiency of DNA replication in a chromatin-specific manner. Genes & Dev. 14, 1308-1312.
Allmang, C., Petfalsid, E., Podtelejnikov, A., Mann, M., Tollervey, D., and Mitchell, P. (1999). The yeast exosome and human PM-Scl are related complexes of 3′→5′ exonucleases. Genes Dev 13, 2148-2158.
Bachi, A., Braun, I. C., Rodrigues, J. P., Pante, N., Ribbeck, K., von Kobbe, C., Kutay, U., Wilm, M., Görlich, D., Carmo-Fonseca, M., and Izaurralde, E. (2000) The C-terminal domain of TAP interacts with the nuclear complex and promotes export of specific CTE-bearing RNA substrates. RNA, 6, 136-158.
Bashkirov, V. I., Scherthan, H., Solinger, J. A., Buerstedde, J. M., and Heyer, W. D. (1997). A mouse cytoplasmic exoribonuclease (mXRN1p) with preference for G4 tetraplex substrates. J Cell Biol 136, 761-773.
Belgrader P., Cheng, J., Zhou, X., Stephenson, L. S., and Maquat, L. E. (1994). Mammalian nonsense codons can be cis-effectors of nuclear mRNA half-life. Mol. Cell. Biol. 14, 8219-8228.
Belgrader, P., and Maquat, L. E. (1994) Nonsense but not missense mutations can decrease the abundance of nuclear mRNA for the mouse major urinary protein, while both types of mutations can facilitate exon skipping. Mol. Biol. Cell, 14, 6326-6336.
Belgrader P., Cheng, J., and Maquat, L. E. (1993). Evidence to implicate translation by ribosomes in the mechanism by which nonsense codons reduce the nuclear level of human triosephosphate isomerase. Proc. Natl. Acad. Sci, USA. 90, 482-486.
Bhattacharya, A., Czaplinski, K., Trifillis, P., He, F., Jacobson, A., and Peltz, S. W. (2000). Characterization of the biochemical properties of human Upf1 gene product that is involved in nonsense-mediated mRNA decay. RNA 6, 1226-1235.
Bienroth, S., Keller, W., and Wahle, E. (1993). Assembly of a processive messenger RNA polyadenylation complex. EMBO J. 12, 585-594.
Blencowe, B. J., Issner, R., Nickerson, J. A. and Sharp, P. A. (1998) A coactivator of pre-mRNA splicing. Genes & Dev., 12, 996-1009.
Brouwer, R., Allmang, C., Raijmakers, R., van Aarssen, Y., Egberts, W. V., Petfalski, E., van Venrooij, W. J., Tollervey, D., and Pruijn, G. J. (2001). Three novel components of the human exosome. J Biol Chem 276, 6177-6184.
Cao, D., and Parker, R. (2003). Computational modeling and experimental analysis of nonsense-mediated decay in yeast. Cell 113, 533-545.
Carter, M. S., Li, S., and Wilkinson, M. F. (1996). A splicing-dependent regulatory mechanism that detects translation signals. EMBO J. 15, 5965-5975.
Castanotto, D., Li, H., and Rossi, J. J. (2002). Functional siRNA expression from transfected PCR products. RNA 8, 1454-1460.
Chen, C. Y., Gherzi, R., Ong, S. E., Chan, E. L., Raijmakers, R., Pruijn, G. J., Stoecklin, G., Moroni, C., Mann, M., and Karin, M. (2001). AU binding proteins recruit the exosome to degrade ARE-containing mRNAs. Cell 107, 451-464.
Cheng, J., and Maquat, L. E. (1993). Nonsense codons can reduce the abundance of nuclear mRNA without affecting the abundance of pre-mRNA or the half-life of cytoplasmic mRNA. Mol. Cell. Biol. 13, 1892-1902.
Chu, E., Schmitz, J. C., Ju. J., and Copur, S. M. (1999). An immunoprecipitation-RNA:rPCR method for the in vivo isolation of ribonucleoprotein complexes. In RNA-Protein Interaction Protocols, Haynes, S. R. (Totowa, N.J.: Humana Press), pp 265-274.
Culbertson, M. R. (1999) RNA surveillance. Unforeseen consequences for gene expression, inherited genetic disorders and cancer. Trends Genet., 15, 74-80.
Czaplinski, K., Ruiz-Echevarria, M. L., Paushkin, S. V., Han, X., Weng, Y., Perlick H. A., Dietz, H. C., Ter-Avanesyan, M. D., and Peltz, S. W. (1998) The surveillance complex interacts with the translation release factors to enhance termination and degrade aberrant mRNAs. Genes & Dev., 12, 1655-1677.
Daneholt, B. (1997) A look at messenger RNP moving through the nuclear pore. Cell, 88, 585-588.
Das, B., Guo, Z., Russo, P., Chartrand, P., and Sherman F. (2000). The role of nuclear ca [binding protein Cbc1p of yeast in mRNA termination and degradation. Mol. Cell. Biol. 20, 2827-2838.
Dehlin, E., Wormington, M., Korner, C. G., and Wahle, E. (2000). Cap-dependent deadenylation of mRNA. EMBO J 19, 1079-1086.
Denning, G., Jamieson, L., Maquat, L. E., Thompson, E. A. and Fields, A. P. (2001) Cloning of a novel phosphoinositide kinase-related kinase: characterization of the human SMG-1 RNA surveillance protein. J. Biol. Chem., 276, 22709-22714. Dostie, J, Lejbkowicz, F. and Sonenberg, N. (2000) Nuclear eukaryotic initiation factor 4E (eIF4E) colocalizes with splicing factors in speckles. J. Cell Biol., 148, 239-247.
Dreyfuss, G., Kim, V. N., and Kataoka, N. (2002). Messenger-RNA-binding proteins and the messages they carry. Nat Rev Mol Cell Biol 3, 195-205.
Dubois, M. F., Nguyen, V. T., Dahmus, M. E., Pages, G., Pouyssegur, J. and Bensaude, O. (1994) Enhanced phosphorylation of the C-terminal domain of RNA polymerase II upon serum stimulation of quiescent cells: possible involvement of MAP kinases. EMBO J, 13, 4787-4797.
Faulkner, N. E., Hilfinger, J. M. and Markovitz, D. M. (2001) Protein phosphatase 2A activates the HIV-2 promoter through enhancer elements that include the pets site. J. Biol. Chem., 276, 25804-25812.
Fortes, P., Inada, T., Preiss, T., Hentze, M. W., Mattaj, I. W., and Sachs, A. B. (2000). The yeast nuclear cap binding complex can interact with translation factor eIF4G and mediate translation intiation. Mol. Cell 6, 191-196.
Frischmeyer, P. A. and Dietz, H. C. (1999). Nonsense-mediated mRNA decay in health and disease. Hum. Mol. Genet. 8, 893-1900.
Gatfield, D., Le Hir, H., Schmitt, C., Braun, I. C., Kocher, T., Wilm, M. and Izaurralde, E. The DexH/D-box protein HEL/UAP65 is essential for mRNA nuclear export in Drosophila. Curr. Biol, 11, 1716-1721.
Gatfield, D. et al. (2003) Nonsense-mediated mRNA decay in Drosophila: at the intersection of the yeast and mammalian pathways. EMBO. J, 22, 3960-3970.
Gao, M., Fritz, D. T., Ford, L. P., and Wilusz, J. (2000). Interaction between a poly(A)-specific ribonuclease and the 5′ cap influences mRNA deadenylation rates in vitro. Mol Cell 5, 479-488.
Gehring, N. H at al. (2003) Y14 and hUpf3b form an NMD-activating complex. Mol Cell. 2003 11:939-49.
Gingras, A.-C., Raught, B. and Sonenberg, N. (1999) eIF4 initiation factors: effectors of mRNA recruitment to ribosomes and regulators of translation. Ann. Rev. Biochem., 68, 913-963.
Hagan, K. W., Ruiz-Echevarria, M. J., Quan, Y., and Peltz, S. W. (1995). Characterization of cis-acting sequences and decay intermediates involved in nonsense-mediated mRNA turnover. Mol Cell Biol 15, 809-823.
Harlowe, E. and Lane, D. (1999). Using Antibodies: A Laboratory Manual. (Cold Spring Harbor, N.Y.: Cold Spring Harbor Laboratory Press), pp. 74-76.
He, F., and Jacobson, A. (1995). Identification of a novel component of the nonsense-mediated mRNA decay pathway by use of an interacting protein screen. Genes Dev 9, 437-454.
Hentze, M. W., and Kulozik, A. E. (1999) A perfect mRNA: RNA surveillance and nonsense-mediated decay. Cell, 96, 307-310.
Hentze, M. W. (1997). eIF4G: A multipurpose ribosome adapter? Science 275, 500-501.
Hirose, Y., and Manley, J. L. (2000) RNA polymerase II and the integration of nuclear events. Genes & Dev. 14, 1415-1429.
Huang, Y., and Steitz, J. A. (2001) Splicing factors SRp20 and 9G8 promote the nucleocytoplasmic export of mRNA. Mol. Cell., 7, 899-905.
Inataka, H., Gradi, A., and Sonenberg, N. (1998). A newly identified N-terminal amino acid sequence of human eIF4G binds poly(A) binding protein and functions in poly(A)-dependent translation. EMBO J.17, 7480-7489.
Ingelfinger, D., Arndt-Jovin, D. J., Luhrmann, R., and Achsel, T. (2002). The human LSm1-7 proteins colocalize with the mRNA-degrading enzymes Dcp1/2 and Xrn1 in distinct cytoplasmic foci. RNA 8, 1489-1501.
Ishigaki, Y., Li, X., Serin, G. and Maquat, L. E. (2001) Evidence for a pioneer round of translation: mRNAs subject to nonsense-mediated decay in mammalian cells are bound by CBP80 and CBP20. Cell, 106, 607-617.
Izaurralde, E., Lewis, J., Gamberi, C., Jarmolowski, A., McGuigan, C., and Mattaj, I. W. (1995). A cap-binding protein complex mediating U snRNA export. Nature 376, 709-712.
Izaurralde, E., Lewis, J., McGuigan, C., Jankowska, M., Darzynkiewicz, E. and Mattaj, I. W. (1994) A nuclear cap binding protein complex involved in pre-mRNA splicing. Cell, 78, 657-668.
Izaurralde, E., Stepinski, J., Darzynlkiewicz, E, and Mattaj, I. W. (1992) A cap binding protein that may mediate nuclear export of RNA polymerase II-transcribed RNAs. J. Cell Biol., 118, 1287-1295.
Jacobson, A., and Peltz, S. W. (2000). Destabilization of nonsense-containing transcripts in Saccharomyces cerevisiae. In Translational Control of Gene Expression, Sonenberg N, Hershey J B W, Mathews M B, eds. (Cold Spring Harbor, N.Y.: Cold Spring Harbor Press), pp 827-847.
Katahira, J., Sträβer, K., Podtelejnikov, A., Mann, M., Jung, J. U., and Hurt, E. (1999) The Mex67p-mediated nuclear mRNA export pathway is conserved from yeast to human. EMBO J., 18, 2593-2609.
Kataoka, N., Yong, J., Kim, V. N., Velazquez, F., Perkinson, R. A., Wang, F. and Dreyfuss, G. (2000) Pre-mRNA splicing imprints mRNA in the nucleus with a novel RNA-binding protein that persists in the cytoplasm. Mol Cell, 6, 673-682.
Kataoka, N., Diem, M. D., Kim, V. N., Yong, J. and Dreyfuss, G. (2001) Magoh, a human homolog of Drosophila mago nashi protein, is a component of the splicing-dependent exon-exon junction complex. EMBO J., 20: 6424-6433.
Kessler, S. H., and Sachs, A. B. (1998). RNA recognition motif 2 of yeast Pab1p is required for its functional interaction with eukaryotic translation initiation factor 4G. Mol. Cell. Biol. 18, 51-57.
Kim, V. N., Kataoka, N. And Dreyfuss, G. (2001a) Role of nonsense-mediated decay factor Upf3 in the splicing-dependent exon-exon junction complex. Science, 293, 1832-1836
Kim, V. N. Yong, J., Kataoka, N., Abel, L., Diem M. D. and Dreyfuss, G. (2001b) The Y14 protein communicates to the cytoplasm the position of exon-exon junctions. EMBO J., 20, 2062-2068.
Kim, Y. K., Lee, S. H., Kim, C. S., Seol, S. K., and Jang, S. K. (2003). Long-range RNA-RNA interaction between the 5′ nontranslated region and the core-coding sequences of hepatitis C virus modulates the IRES-dependent translation. RNA 9, 599-606.
Komer, C. G., and Wable, E. (1997). Poly(A) tail shortening by a mammalian poly(A)-specific 3′-exoribonuclease. J Biol Chem 272, 10448-10456.
Komer, C. G., Wormington, M., Muckenthaler, M., Schneider, S., Dehlin, E., and Wahle, E. (1998). The deadenylating nuclease (DAN) is involved in poly(A) tail removal during the meiotic maturation of Xenopus oocytes. EMBO J 17, 5427-5437.
Krause, S., Fakan, S., Weis, K., and Wahle, E. (1994). Immunodetection of poly(A) binding protein II in the cell nucleus. Exp. Cell Res. 214, 75-82.
Kugler, W., Enssle, J., Hentze, M. W. and Kulozik, A. E. (1995). Nuclear degradation of nonsense-mutated ∃-globin mRNA: a post-transcriptional mechanism to protect heterozygotes from severe clinical manifestations of ∃-thalassemia? Nuc. Acids Res. 23, 413-418.
Lejbkowicz, F., Goyer, C., Darveau, A., Neron, S., Lemieux, R. and Sonenberg, N. (1992) A fraction of the mRNA 5′ cap-binding protein, eukaryotic initiation factor 4E, localizes to the nucleus. Proc. Natl. Acad. Sci., USA, 89, 9612-9616.
Le Hir, H., Izaurralde, E., Maquat, L. E., and Moore, M. J. (2000a) The spliceosome deposits multiple proteins 20-24 nucleotides upstream of mRNA exon-exon junctions. EMBO J., 19, 6860-6869.
Le Hir, H., Moore, M. J. and Maquat, L. E. (2000b) Pre-mRNA splicing alters mRNP composition: evidence for a stable association of proteins at exon-exon junctions. Genes & Dev., 14, 1098-1108.
Le Hir, H., Gatfield, D., Izaurralde, E. and Moore, M. J. (2001 a) The exon-exon junction complex provides a binding platform for factors involved in mRNA export and NMD. EMBO J., 17, 4987-4997.
LeHir, H., Gatfield, D., Braun, I. C., Forler, D. and Izaurralde, E. (2001b) The protein Mago provides a link between splicing and mRNA localization. EMBO Rep., 21:1119-1124.
Le Hir, H., Nott, A., and Moore, M. J. (2003). How introns influence and enhance eukaryotic gene expression. Trends in Biochemical Sciences. 28(4)215-220.
Lejeune, F., Ishigaki, Y., Li, X., and Maquat, L. E. (2002) The exon junction complex is detected on CBP80-bound but not eIF4E-bound mRNA in mammalian cells: Dynamics of mRNP remodeling. EMBO J. 21: 3536-3545.
Lewis, J. D. and Izaurralde, E. (1997) The role of the cap structure in RNA processing and nuclear export. Eur. J. Biochem., 247, 461-469.
Li, S. and Wilkinson, M. F. (1998) Nonsense surveillance in lymphocytes? Immunity, 8, 135-141.
Losson, R., and Lacroute, F. (1979). Interference of nonsense mutations with eukaryotic messenger RNA stability. Proc Natl Acad Sci USA 76, 5134-5137.
Lou, M. J. and Reed, R. (1999) Splicing is required for rapid and efficient mRNA export in metazoans. Proc. Natl. Acad. Sci., USA, 96, 14937-14942.
Lou, M. J., Zhou, Z., Magni, K., Christoforides, C., Rappsilber, J. Mann, M. and Reed, R. (2001) Pre-mRNA splicing and mRNA export linked by direct interactions between UAP56 and Aly. Nature, 413, 644-647.
Lykke-Andersen, J., Shu, M-D. and Steitz, J. A. (2000) Human Upf proteins target an mRNA for nonsense-mediated decay when bound downstream of a termination codon. Cell, 103, 1121-1131.
Lykke-Andersen, J., Shu, M.-D. and Steitz, J. A. (2001) The protein RNPS1 communicates the position of exon-exon junctions to the mRNA surveillance machinery. Science, 293, 1836-1839.
Lykke-Andersen, J. (2002). Identification of a human decapping complex associated with hUpf proteins in nonsense-mediated decay. Mol Cell Biol 22, 8114-8121.
McCracken, S., Fong, N., Rosonina, E., Yankulov, K., Brothers, G., Siderovski, D., Hessel, A., Foster, S., Shuman, S., Bentley, D. L. (1997) 5′-Capping enzymes are targeted to pre-mRNA by binding to the phosphorylated carboxy-terminal domain of RNA polymerase II. Genes & Dev., 11, 3306-3318.
McCracken, S., Lambermon, M. and Blencowe, B. J. (2002) SRm160 splicing coactivator promotes transcript 3′-end cleavage. Mol. Cell. Biol., 22, 148-160.
McGarvey, T., Rosonina, E., McCracken, S., Li, Q., Araout, R., Mientjes, E., Nickerson, J. A., Awrey, D., Greenblatt, J., Grosveld, G. and Blencowe, B. J. (2000) The acute myeloid leukemia-associated protein DEK forms a splicing-dependent interaction with exon-exon product complexes. J. Cell. Biol., 150, 309-320.
McKendrick, L., Thompson, E., Ferreira, J., Morley, S. J., and Lewis, J. D. (2001) Interaction of eukaryotic translation initiation factor 4G with the nuclear cap-binding complex provides a link between nuclear and cytoplasmic functions of the m(7) guanosine cap. Mol. Cell. Biol. 21, 3632-3641.
Maderazo, A. B., He, F., Mangus, D. A. and Jacobson, A. (2000) Upf1p control of nonsense mRNA translation is regulated by Nmd2p and Upf3p. Mol. Cell. Biol., 20, 4591-4603.
Maderazo, A. B., Belk, J. P., He, F., and Jacobson, A. (2003). Nonsense-containing mRNAs that accumulate in the absence of a functional nonsense-mediated mRNA decay pathway are destabilized rapidly upon its restitution. Mol Cell Biol 23, 842-851.
Maquat, L. E. (2002) Nonsense-mediated mRNA decay. Curr. Biol., 12: R196-197.
Maquat, L. E. and Carmichael, G. G. (2001) Quality control of mRNA function. Cell, 104, 173-176.
Maquat, L E., and Serin, G. (2001). Nonsense-mediated mRNA decay: Insights into mechanism from the cellular abundance of human Upf1, Upf2, Upf3 and Upf3X proteins. Cold Spring Harbor Symposia on Quantitative Biology: The Ribosome. (Cold Spring Harbor, N.Y.: Cold Spring Harbor Press), 66: 313-320.
Maquat, L. E. (2000) Nonsense-mediated RNA decay in mammalian cells: a splicing-dependent means to down-regulated the levels of mRNAs that prematurely terminate translation. In Translational Control of Gene Expression, Sonenberg N, Hershey J B W, Mathews M B, eds. (Cold Spring Harbor, N.Y.: Cold Spring Harbor Press), pp 849-868.
Maquat, L. E. (1995) When cells stop making sense: Effects of nonsense codons on RNA metabolism in vertebrate cells. RNA, 1, 453-465.
Maquat, L. E. (2003) Nonsense-mediated mRNA decay in mammalian cells: From pre-processing to mRNA translation and degradation. In: Translation Mechanisms, ed Jacques Lapointe and Lea Brakier-Gingras, Landes Bioscience, Austin, Tex., pp. 213-222.
Marcotrigiano, J., Gingras, A.-C., Sonenberg, N., and Burley, S. K. (1999). Cap-dependent translation initiation in eukaryotes is regulated by a molecular mimic of eIF4G. Mol. Cell 3, 1-20.
Mayeda, A., Badolato, J., Kobayashi, R., Zhang, M. Q., Gardiner, E. M. and Krainer, A. R. (1999) Purification and characterization of human RNPS1: a general activator of pre-mRNA splicing. EMBO J., 18, 4560-4570.
Mehlin, H., Daneholt, B. and Skoglund, U. (1992) Translocation of a specific premessenger ribonucleoprotein particle through the nuclear pore studied with electron microscope tomography. Cell, 69, 605-613.
Mendell, J. T., Medghalchi, S. M., Lake R. G., Noensie, E. N., and Dietz, H. C. (2000) Novel Upf2p orthologues suggest a functional like between translation initiation and nonsense surveillance complexes. Mol. Cell. Biol., 20, 8944-8957.
Mendell, J. T., ap Rhys, C. M., and Dietz, H. C. (2002). Separable roles for rent1/hUpf1 in altered splicing and decay of nonsense transcripts. Science 298, 419-422.
Mendell, J. T., and Dietz, H. C. (2001). When the message goes awry: disease-producing mutations that influence mRNA content and performance. Cell 107, 411-414.
Mitchell, P., and Tollervey, D. (2000). Musing on the structural organization of the exosome complex. Nat Struct Biol 7, 843-846.
Mitchell, P., and Tollervey, D. (2001). mRNA turnover. Curr Opin Cell Biol 13, 320-325.
Mitchell, P., and Tollervey, D. (2003). An NMD pathway in yeast involving accelerated deadenylation and exosome-mediated 3′→5′ degradation. Mol Cell 11, 1405-1413.
Moriarty, P. M., Reddy, C. C., and Maquat, L. E. (1998). Selenium deficiency reduces the abundance of mRNA for Se-dependent glutathione peroxidase 1 by a UGA-dependent mechanism likely to be nonsense codon-mediated decay of cytoplasmic mRNA. Mol. Cell. Biol. 18, 2932-2939.
Morley, S. J., and McKendrick, L. (1997) Involvement of stress-induced protein kinase and p38/RK mitogen-activated protein kinase signaling pathways in the enhanced phosphorylation of initiation factor 4E in NIH 3T3 cells. J. Biol. Chem., 272, 17887-1793.
Muhlrad, D., and Parker, R. (1994). Premature translational termination triggers mRNA decapping. Nature 370, 578-581.
Muhlrad, D., and Parker, R. (1999). Aberrant mRNAs with extended 3′ UTRs are substrates for rapid degradation by mRNA surveillance. RNA 5, 1299-1307.
Nagy, E., and Maquat, L. E. (1998). A rule for termination codon position within intron-containing genes: when nonsense affects RNA abundance. Trends Biol Sci 23, 198-199.
Nemeth, A., Krause, S., Blank, D., Jenny, A., Jeno, P., Lustig, A., and Wahle, E. (1995). Isolation of genomic and cDNA clones encoding bovine poly(A) binding protein II. Nuc. Acids Res. 23, 4034-4041.
Ohno, M., Kataoka, N., and Simura, Y. (1990) A nuclear cap binding protein from HeLa cells. Nuc. Acids Res., 18, 6989-6995.
Orphanides, G., and Reinberg, D. (2000). RNA polymerase II elongation through chromatin. Nature 407, 471-475.
Page, M. F., Carr, B., Anders, K. R, Grimson, A. and Anderson, P. (1999). SMG-2 is a phosphorylated protein required for mRNA surveillance in Caenorhabditis elegans and related to Upf1p of yeast. Mol Cell Biol 19, 5943-51.
Pal, M., Ishigaki, Y., Nagy, E., and Maquat, L. E. (2001) Evidence that phosphorylation of human Upf1 protein varies with intracellular location and is mediated by a wortmannin-sensitive and rapamycin-sensitive PI 3-kinase-related kinase signaling pathway. RNA, 7, 5-15.
Pei, Y., Hausmann, S., Ho, C. K., Schwer, B., and Shuman, S. (2001) The length, phosphorylation state, and primary structure of the RNA polymerase II carboxyl-terminal domain dictate interactions with mRNA capping enzymes. J. Biol. Chem., 276, 28075-28082.
Peltz, S. W., Brown, A. H., and Jacobson, A. (1993). mRNA destabilization triggered by premature translational termination depends on at least three cis-acting sequence elements and one trans-acting factor. Genes Dev 7, 1737-1754.
Peltz, S. W., and Jacobson, A. (2000). Destabilization of nonsense-containing transcripts in Saccharomyces cerevisiae. In Translational control of gene expression, Sonenberg, N., Hershey, J. B. W., Mathews, M. B., ed. (Cold Spring Harbor, N.Y., Cold Spring Harbor Laboratory Press), pp. 827-847.
Proudfoot, N. (2000) Connecting transcription to messenger RNA processing. Trends Biochem. Sci., 25, 290-293.
Ptushkina, M., von der Haar, T., Vasilescu, S., Frank, R., Birkenhager, R, and McCarthy, J. E. G. (1998). Repressor binding to a dorsal regulatory site traps human eIF4E in a high cap-affinity state. EMBO J. 17, 4798-4808.
Pyronnet, S., Imataka, H., Gingras, A.-C., Fukunaga, R., Hunter, T., and Sonenberg, N. (1999). Human eukaryotic translation initiation factor 4G (eIF4G) recruits Mnk1 to phosphorylate eIF4E. EMBO J. 18, 270-279.
Raijmakers, R., Egberts, W. V., van Venrooij, W. J., and Prajn, G. J. (2002). Protein-protein interactions between human exosome components support the assembly of RNase PH-type subunits into a six-membered PNPase-like ring. J Mol Biol 323, 653-663.
Rajavel, K. S., and Neufeld, E. F. (2001). Nonsense-mediated decay of HexA mRNA. Mol. Cell. Biol. 16, 5512-5517.
Reed, R. and Hurt, E. (2002) A conserved mRNA export machinery coupled to pre-mRNA splicing. Cell, 108,523-531.
Reed, R. and Magni, K. (2001) A new view of mRNA export: Separating the wheat from the chaff. Nat. Cell Biol., 3, 201-204.
Rodrigues, J. P., Rode, M., Gatfield, D., Blencowe, B. J. Carmo-Fonseca, M., and Izaurralde, E. (2001) REF proteins mediate the export of spliced and unspliced mRNAs from the nucleus. Proc. Natl. Acad. Sci., USA, 98, 1030-1035.
Sach, A. B. and Varani, G. (2000). Eukaryotic translation initiation: there are (at least) two sides to every story. Nature Struct. Biol. 7, 356-361.
Schoenberg, D. R., and Chernokalskaya, E. (1997). In mRNA metabolism and post-transcriptional gene regulation, J. Harford, and D. R. Morris, eds. (Wiley, N.Y.), pp. 217-240.
Schroeder, S. C., Schwer, B., Shuman, S., and Bentley, D. L. (2000) Dynamic association of capping activities with transcribing RNA polymerase II. Genes & Dev., 14, 2435-2440.
Serin, G., Gersappe, A., Black, J. D., Aronoff, R. and, Maquat, L. E. (2001) Identification and characterization of human orthologues to Saccharornyces cerevisiae Upf2 protein and Upf3 protein (Caenorhabditis elegans SMG-4). Mol. Cell Biol. 21, 209-223.
Shen, E.C., Stage-Zimmermann, T., Chui, P. and Silver, P. A. (2000) The yeast mRNA-binding protein Np13p interacts with the cap-binding complex. J. Biol. Chem., 275, 23718-23724.
Sheth, U., and Parker, R. (2003). Decapping and decay of messenger RNA occur in cytoplasmic processing bodies. Science 300, 805-808.
Shyu, A. B., Belasco, J. G., and Greenberg, M. E. (1991). Two distinct destabilizing elements in the c-fos message trigger deadenylation as a first step in rapid mRNA decay. Genes Dev 5, 221-231.
Sträβer, K. and Hurt, E. (2001) Splicing factor Sub2p is required for nuclear mRNA export through its interaction with Yralp. Nature, 413, 648-652.
Stutz, F., Bachi, A., Doerks, T., Braun, I. C., Seraphin, B., Wilm, M., Bork, P. and Izaurralde, E. (2000) REF, an evolutionary conserved family of hnRNP-like proteins, interactions with TAP/Mex67p and participates in mRNA nuclear export. RNA, 6, 638-650.
Sun, X., Moriarty, P. M., and Maquat, L. E. (2000). Nonsense-mediated decay of glutathione peroxidase 1 mRNA in the cytoplasm depends on intron position. EMBO J. 19, 4734-4744.
Sun, X., Perlick, H. A., Dietz, H. C., and Maquat, L. E. (1998) A mutated human homologue to yeast Upf1 protein has a dominant-negative effect on the decay of nonsense-containing mRNAs in mammalian cells. Proc. Nat. Acad Sci. USA, 95, 10009-10014.
Thermann, R., Neu-Yilik, G., Deters, A., Frede, U., Wehr, K., Hagenmeier, C., Hentze, M. W., and Kulozik, A. E. (1998). Binary specification of nonsense codons by splicing and cytoplasmic translation. EMBO J. 17, 3484-3494.
Tucker, M., and Parker, R. (2000). Mechanisms and control of mRNA decapping in Saccharomyces cerevisiae. Annu Rev Biochem 69, 571-595.
Van Dijk, E., Cougot, N., Meyer, S., Babajko, S., Wahle, E., and Seraphin, B. (2002). Human Dcp2: a catalytically active mRNA decapping enzyme located in specific cytoplasmic structures. EMBO J 21, 6915-6924.
van Hoof, A., and Parker, R. (2002). Messenger RNA degradation: beginning at the end. Curr Biol 12, R285-287.
Visa, N., Izaurralde, E., Ferreira, J., Daneholt, B. and Mattaj, I. W. (1996) A nuclear cap-binding complex binds Balbiani ring pre-mRNA cotranscriptionally and accompanies the ribonucleoprotein particle during nuclear export. J. Cell Biol., 133, 5-14.
Wagner, E., and Lykke-Andersen, J. (2002). mRNA surveillance: the perfect persist. J Cell Sci 115, 3033-3038.
Wahle, E. (1991). A novel poly(A)-binding protein acts as a specificity factor in the second phase of messenger RNA polyadenylation. Cell 66, 759-768.
Wahle, E., Lustig, A., Jeno, P., and Maurer, P. (1993). Mammalian poly(A)-binding protein II. Physical properties and binding to polynucleotides. J. Biol. Chem. 268, 2937-2945.
Wang, W., Czaplinski, K., Rao, Y. and Peltz, S. W. (2001) The role of Upf proteins in modulating the translation read-through of nonsense-containing transcripts. EMBO J., 20, 880-890.
Wang, J., Gudikote, J. P., Olivas, O. R. and Wilkinson, M. F. (2002) Boundary-independent polar nonsense-mediated decay. EMBO Rep., 3, 274-279.
Wang, Z., Jiao, X., Carr-Schmid, A., and Kiledjian, M. (2002). The hDcp2 protein is a mammalian mRNA decapping enzyme. Proc Natl Acad Sci USA 99, 12663-12668.
Weng, Y., Czaplinski, K., and Peltz, S. W. (1996). Genetic and biochemical characterization of mutations in the ATPase and helicase regions of the Upf1 protein. Mol Cell Biol 16, 5477-5490.
Wigler, M., Sweet, R., Sim, G. K., Wold, B., Pellicer, A., Lacy, E., Maniatis, T., Silverstein, S., and Axel, R. (1979). Transformation of mammalian cells with genes from procaryotes and eucaryotes. Cell 16, 777-785.
Wilson, G. M., and Brewer, G. (1999). The search for trans-acting factors controlling messenger RNA decay. Prog Nucleic Acid Res Mol Biol 62, 257-291.
Wilusz, C. J., Wang, W., and Peltz, S W. (2001) Curbing the nonsense: the activation and regulation of mRNA surveillance. Genes & Dev., 15:2781-2785.
Yamashita, A., Ohnishi, T., Kashima, I., Taya, Y. and Ohno, S. (2001) Human SMG-1, a novel phosphatidylinositol 3-kinase-related protein kinase, associates with components of the mRNA surveillance complex and is involved in the regulation of nonsense-mediated mRNA decay. Genes & Dev., 15:2215-2228.
Zhang, J., Sun, X., Qian, Y. and Maquat, L. E. (1998) Intron function in the nonsense-mediated decay of beta-globin mRNA: indications that pre-mRNA splicing in the nucleus can influence mRNA translation in the cytoplasm. RNA, 4, 801-815.
Zhang, J., Sun, X., Qian, Y, LaDuca, J. P., and Maquat, L. E. (1998b). At least one intron is required for the nonsense-mediated decay of triosephosphate isomerase mRNA: a possible link between nuclear splicing and cytoplasmic translation. Mol. Cell. Biol. 18, 5272-5283.
Zhang, M., Yu, L., Xin, Y., Hu, P., Fu, Q., Yu, C., and Zhao, S. (1999). Cloning and mapping of the XRN2 gene to human chromosome 20p11.1-p11.2. Genomics 59, 252-254.
Zhang, S., Welch, E. M., Hogan, K., Brown, A. H., Peltz, S. W., and Jacobson, A. (1997). Polysome-associated mRNAs are substrates for the nonsense-mediated mRNA decay pathway in Saccharomyces cerevisiae. RNA 3, 234-244.
Zhou, Z., Lou, M. J., Staesser, K., Datahira, J., Hurt, E., and Reed, R. (2000) The protein Aly links pre-messenger-RNA splicing to nuclear export in metazoans. Nature, 407, 401-407.

What is claimed is:

1. A method of treating a disorder in a subject comprising administering to the subject a substance, wherein the substance modulates of nonsense-mediated mRNA decay in the pioneer round of translation.

2. The method of claim 1, wherein the disorder is an inherited genetic disorder.

3. The method of claim 2, wherein the genetic disorder can be selected from the group consisting of cystic fibrosis, hemophilia, mucopolysaccharidoses, muscular dystrophy, anemia, glycolytic enzyme deficiency, connective tissue disorder, DNA repair disorder, dementia, Sandhoff disease, epidermolysis bullosa simplex, insulin resistance, maple syrup urine disease, hereditary fructose intolerance, inherited immunodeficiency, inherited cancer, carbohydrate metabolism disorder, amino acid metabolism disorder, lipoprotein metabolism disorder, lipid metabolism disorder, lysomal enzymes disorder, steroid metabolism disorder, purine metabolism disorder, pyrimidine metabolism disorder, metal metabolism disorder, porphyrin metabolism disorder, and heme metabolism disorder.

4. The method of claim 1, wherein the disorder is an acquired disorder.

5. The method of claim 4, wherein the acquired disorder is a mutation in p53.

6. The method of claim 4, wherein the acquired disorder is a mutation in BRCA-1.

7. The method of claim 4, wherein the acquired disorder is a cancer.

8. The method of claim 7, wherein the cancer is selected from the group consisting of lymphoma, B cell lymphoma, T cell lymphoma, leukemia, carcinoma, sarcoma, glioma, blastoma, neuroblastoma, plasmacytoma, histiocytoma, melanoma, adenoma, mycosis fungoide, hypoxic tumor, myeloma, metastatic cancer, bladder cancer, brain cancer, nervous system cancer, head and neck cancer, ovarian cancer, pancreatic cancer, prostate cancer, skin cancer, liver cancer, colon cancer, cervical cancer, cervical carcinoma, breast cancer, epithelial cancer, renal cancer, genitourinary cancer, pulmonary cancer, esophageal carcinoma, hematopoietic cancers, testicular cancer, colorectal cancer, and prostatic cancer.

9. The method of claim 1, wherein the subject is a mammal.

10. The method of claim 1, wherein the modulation is a decrease in nonsense-mediated mRNA decay.

11. The method of claim 1, wherein the modulation is an increase in nonsense-mediated mRNA decay.

12. The method of claim 1, wherein the substance interacts with Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, or Rrp41.

13. The method of claim 1, wherein the substance is a functional nucleic acid, siRNA, peptide, protein, antibody, or chemical.

14. A method of screening for a substance that modulates a nonsense-mediated mRNA decay (NMD) complex comprising
(a) incubating the substance with the complex, and
(b) assaying for a change in NMD, an increase or decrease in NMD activity indicating a modulating substance.

15. The method of claim 14, wherein the complex comprises Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

16. The method of claim 14, wherein the complex comprises one or more of the group consisting of Upf2, Upf3, Upf3X, PABP2, CBP20, CBP80, hSmg5/7a, hSmg5/7b, hsmg5Sc, Dcp2, PM/Scl100, poly (A) ribonuclease (PARN), Rat1, Xrn1, and Rrp41.

17. The method of claim 14, wherein the complex comprises at least two members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

18. The method of claim 14, wherein the complex comprises at least three members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

19. The method of claim 14, wherein the complex comprises at least four members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

20. The method of claim 14, wherein the complex comprises at least five members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

21. The method of claim 14, wherein the complex comprises at least six members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

22. The method of claim 14, wherein the complex comprises at least seven members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

23. The method of claim 14, wherein the complex comprises at least eight members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

24. The method of claim 14, wherein the complex comprises at least nine members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

25. The method of claim 14, wherein the complex comprises at least ten members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

26. The method of claim 14, wherein the complex comprises at least eleven members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

27. The method of claim 14, wherein the complex comprises at least twelve members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

28. The method of claim 14, wherein the complex comprises at least thirteen members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

29. The method of claim 14, wherein the complex comprises at least fourteen members of the group consisting of Upf2, Upf3, Upf3X, PABP2, CBP20, CBP80, hSmg5/7a, hSmg5/7b, hSmg5/7c, Dcp2, PM/Scl100, poly (A) ribonuclease (PARN), Rat1, Xrn1, and Rrp41.

30. A method of screening for a substance that modulates nonsense-mediated mRNA decay (NMD) comprising
(a) incubating the substance with a stably transfected cell comprising a reporter gene with a nonsense-mutation, and
(b) assaying the amount of NMD in the cell, wherein a increase or decrease in the amount of mRNA relative to the amount of mRNA in the absence of the substance indicates a substance that modulates NMD activity.

31. A method of screening for a substance that inhibits nonsense-mediated mRNA decay (NMD) comprising
(a) incubating the substance with Upf2 and Upf3 forming a substance-Upf2-Upf3 mixture, and
(b) assaying the amount of Upf2-Upf3 complex present in the mixture, wherein a decrease in the amount of Upf2-Upf3 complex relative to the amount of Upf2-Upf3 complex in the absence of the substance indicates that the substance inhibits NMD.

32. The method of claim 31, wherein the Upf2 has at least 80%, 85%, 90%, or 95% identity to the sequence set forth in ATCC No. AF318574, or fragment thereof.

33. The method of claim 31, wherein the Upf3 has at least 80%, 85%, 90%, or 95% identity to the sequence set forth in ATCC No. AF318575, or fragment thereof.

34. A method of screening for a substance that inhibits nonsense-mediated mRNA decay (NMD) comprising
(a) incubating the substance with Upf2 and Upf3X forming a substance-Upf2-Upf3X mixture, and
(b) assaying the amount of Upf2-Upf3X complex present in the mixture, wherein a decrease in the amount of Upf2-Upf3X complex relative to the amount of Upf2-Upf3X complex in the absence of the substance indicates that the substance inhibits NMD.

35. The method of claim 34, wherein the Upf2 has at least 80%, 85%, 90%, or 95% identity to the sequence set forth in ATCC No.:AF318574 or a fragment thereof.

36. The method of claim 34, wherein the Upf3X has at least 80%, 85%, 90%, or 95% identity to the sequence set forth in ATCC No.:AF318576 or a fragment thereof.

37. A method of screening for a substance that modulates nonsense-mediated mRNA decay (NMD) comprising,
a) administering a substance to a system, wherein the system comprises the components essential for NMD activity, and
b) assaying the effect of the substance on the amount of NMD activity in the system, wherein a substance which causes a change in the amount of NMD activity present in the system compared to the amount of NMD activity in the system in the absence of composition is a modulator.

38. A method of modulating nonsense-mediated mRNA decay (NMD) activity comprising administering a substance, wherein the substance is identified by the method of claim 37.

39. A method of making a substance capable of modulating nonsense-mediated mRNA decay (NMD) activity comprising admixing a substance identified by the method of claim 29 with a pharmaceutically acceptable carrier.

40. A substance made by the method of claim 39.

41. A method of making a modulator of nonsense-mediated mRNA decay (NMD) comprising,
a) administering a substance to a system, wherein the system comprises NMD activity,
b) assaying the effect of the substance on the amount of NMD activity in the system,
c) selecting a substance which causes a change in the amount of NMD activity present in the system compared to the amount of NMD activity in the system in the absence of substance, and
d) synthesizing the substance.

42. A substance that modulates NMD in the pioneer round of translation.

43. The substance of claim 42, wherein the substance is an antibody that modulates NMD.

44. The substance of claim 43, wherein the antibody binds Upf2.

45. The substance of claim 43, wherein the antibody binds Upf3.

46. The substance of claim 43, wherein the antibody binds Upf3X.

47. The substance of claim 43, wherein the antibody binds PABP2.

48. The substance of claim 43, wherein the antibody binds PARN.

49. The substance of claim 43, wherein the antibody binds Dcp2.

50. The substance of claim 43, wherein the antibody binds PM/Scl100.

51. The substance of claim 43, wherein the antibody binds Rat1.

52. The substance of claim 43, wherein the antibody binds Xrn1.

53. The substance of claim 43, wherein the antibody binds Rrp41.

54. The substance of claim 43, wherein the antibody binds hSmg5/7a.

55. The substance of claim 43, wherein the antibody binds hSmg5/7b.

56. The substance of claim 43, wherein the antibody binds hSmg5/7c.

57. The substance of claim 42, wherein the substance is a vector comprising a nucleic acid that encodes a protein that modulates NMD.

58. A vector comprising a nucleic acid that encodes a protein that modulates NMD.

59. A cell comprising the vector of claim 57.

60. The substance of claim 42, wherein the substance is siRNA that modulates NMD.

61. The substance of claim 60, wherein the siRNA binds Upf2.

62. The substance of claim 60, wherein the siRNA binds Upf3.

63. The substance of claim 60, wherein the siRNA binds Upf3X.

64. The substance of claim 60, wherein the. siRNA binds PABP2.

65. The substance of claim 60, wherein the siRNA binds PARN.

66. The substance of claim 60, wherein the siRNA binds Dcp2.

67. The substance of claim 60, wherein the siRNA binds PM/Scl100.

68. The substance of claim 60, wherein the siRNA binds Rat1.

69. The substance of claim 60, wherein the siRNA binds Xrn1.

70. The substance of claim 60, wherein the siRNA binds Rrp41.

71. The substance of claim 60, wherein the siRNA binds hSMg5/7a.

72. The substance of claim 60, wherein the siRNA binds hSmg5/7b.

73. The substance of claim 60, wherein the siRNA binds hSmg5/7c.
